(19) United States
(12) Patent Application Publication
(10) Pub. No.: US 2026/0130860 A1
(43) Pub. Date: May 14, 2026

(54) LIPIDS FOR DELIVERY OF THERAPEUTIC AGENTS

(71) Applicant: Flagship Labs 114, Inc., Cambridge, MA (US)

(72) Inventor: Qi-Ying Hu, Cambridge, MA (US)

(73) Assignee: Flagship Labs 114, Inc., Cambridge, MA (US)

(21) Appl. No.: 18/867,152

(22) PCT Filed: May 19, 2023
(86) PCT No.: PCT/US2023/067206

§ 371 Date: Nov. 19, 2024

Related U.S. Application Data
(60) Provisional application No. 63/344,162, filed on May 20, 2022.
(60) Provisional application No. 63/380,214, filed on Oct. 19, 2022.

(51) Int. Cl. A61K 9/51; A61K 31/7105; A61K 47/24; A61K 47/28

(52) U.S. Cl. CPC A61K 9/5123; A61K 9/5192; A61K 31/7105; A61K 47/24; A61K 47/28

(57) ABSTRACT

The current disclosure relates to lipid-based compositions and methods useful in administering therapeutic agents such as, for example, nucleic acids (e.g., siRNA, ASO, tRNA, miRNA, mRNA, DNA, and the like), proteins, peptides, and other macromolecules, which do not have the ability to easily cross a cell membrane. In particular, certain embodiments of the disclosure relate to reversible zwitterionic lipids having a tertiary amine with an increased pKa and extended linker domains (e.g., ≥C3), that may be incorporated into lipid-based compositions (e.g., lipid nanoparticles) to increase efficiency of delivery and release of a therapeutic agent(s) to a subject. The present disclosure provides compositions comprising such reversible zwitterionic lipids, optionally in association with a therapeutic agent (e.g., a therapeutic mRNA and/or nucleic acid controller system), as well as methods of synthesizing the ionizable lipid particle compositions provided herein.

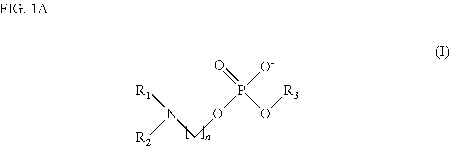

### FIELD

The current disclosure relates to lipid-based compositions and methods useful in administering therapeutic agents. In particular, the disclosure relates to reversible zwitterionic lipids having an ionizable tertiary amine and a phosphate group that together form a zwitterion that is reversible at different pH values, which may be incorporated into lipid-based compositions to increase efficiency of delivery of a therapeutic agent(s) to a subject.

### BACKGROUND

Lipid-based compositions (e.g., lipid nanoparticles (LNPs), cationic liposomes, polymers, and the like) can provide effective drug delivery systems for therapeutic agents such as, for example, nucleic acids (e.g., siRNA, ASO, tRNA, miRNA, mRNA, DNA, and the like), proteins, peptides, and other macromolecules, which do not have the ability to easily cross a cell membrane (e.g., cell impermeable agents). For example, a variety of clinical applications of nucleic acid therapies require delivering nucleic acids to one or more intracellular compartments that contain the RISC complex, a cell's transcriptional machinery and/or genomic DNA, in order to be effective, and a number of LNP formulations have been characterized as able to facilitate such delivery in an effective manner. Additionally, LNP formulations have been shown to shield cargo nucleic acids from degradation when utilized for in vivo delivery (via parenteral or other routes of administration), which can significantly reduce cargo nucleic acid doses needed to achieve cargo nucleic acid expression and/or target modulation/knockdown in vivo in a targeted tissue/cell population.

LNPs currently prevalent in the art generally include four lipid components: cationic lipids, helper lipids, cholesterol, and polyethylene glycol (PEG)-lipids. Positively charged cationic lipids are used to bind to therapeutic agents (e.g., anionic nucleic acids) as cargo, while the other components generally facilitate stable self-assembly of the LNP around the cargo, while preventing aggregates from forming.

An important aspect of the use of LNP-based delivery systems is the ability of the LNP to release its cargo, which generally is believed to occur via endosomal escape. Disadvantageously, even highly effective LNP carriers described in the art (e.g., DLin-MC3-DMA-containing LNPs) are only able to achieve approximately 1-10% release of intracellular RNA into the cytoplasm, and there is a concern that higher doses of certain lipid components of LNPs might cause negative effects (e.g., toxicity) in certain instances.

More recently, multi-tailed ionizable phospholipids (e.g., iPhos) composed of one pH-switchable zwitterion and three hydrophobic tails have been used to facilitate cargo release from endosomes. The small zwitterion constituted by the amine group and the phosphate group of these lipids is predicted to be reversible at different pHs. For example, at physiological pH the tertiary amine group will not be protonated, and the negatively charged phospholipids will have difficulty fusing into the membranes; however, when such phospholipids enter the endosomes, the acidic environment causes the tertiary amine to be protonated to form a zwitterionic head, which in combination with the tri-hydrophobic tail structure mediates membrane phase transformation in a more efficient manner that prior phospholipids consisting of only two tails. Such phospholipid chemical structures can be combined with zwitterionic, ionizable cationic, and permanently cationic helper lipids to facilitate tissue-selective cargo release and delivery. While improvements in this area have been made over recent years, it is clear that achieving greater endosomal escape remains a fundamental barrier in advancing the ability to deliver therapeutic agents such as, for example, nucleic acid therapeutics. There remains an unmet need in the art for safe, efficient, and effective lipids and lipid-based compositions that facilitate delivery of therapeutic agents, particularly nucleic acid cargoes.

### BRIEF SUMMARY

The present disclosure is based, at least in part, upon the discovery of novel reversible zwitterionic lipids having an ionizable tertiary amine that is connected to an electron withdrawing phosphate group via a linker including ≥C₃, which together form a zwitterion that is reversable at different pH values. Additionally, the reversible zwitterionic lipids disclosed herein demonstrate an increased pKa of the ionizable tertiary amine relative to prior art phospholipids (e.g., iPhos) that only have a C₂ linker. Without being bound by theory, it is believed that there is an electrostatic interaction between the ionizable tertiary amine and the phosphate group, and that increasing the length of the linker between the tertiary amine and the phosphate group to ≥C₃ decreases the electrostatic interaction between them, thereby increasing the pKa of the tertiary amine.

Furthermore, the reversible zwitterionic lipids disclosed herein may include an ionizable tertiary amine (e.g., pH-titratable) head group, a linker, and a phosphate group, where the ionizable tertiary amine head group includes two hydrocarbon chains (e.g., C₇-C₂₂ alkyl, alkenyl, or alkynyl) and the phosphate group includes one hydrocarbon chain (e.g., C₃-C₂₂ alkyl, alkenyl, or alkynyl), where each of the aforementioned hydrocarbon chains independently has 0 to 3 (e.g., 0, 1, 2, or 3) double bonds, and ether, ester, or ketal linkages between the ionizable amine head group and hydrocarbon chains. The reversible zwitterionic lipids disclosed herein have advantageous properties when used in lipid particles for the in vivo delivery of a therapeutic agent(s) because the ionizable tertiary amine may become protonated when the reversible zwitterionic lipid enters the endosome to form a zwitterionic head and the three hydrocarbon chains are able to form a cone shaped structure when inserted into the endosomal membrane that facilitates hexagonal transformation. Additionally, the present disclosure provides reversible zwitterionic lipids having an ionizable tertiary amine with an increased pKa relative to prior art lipids that only include a C₂ linker between a tertiary amine and a phosphate group (see e.g., the iPhos chemical structures disclosed in Liu et al. 2021 Nat. Mater. 20(5): 701-710) that can advantageously be incorporated into lipid nanoparticles to improve endosomal escape and thereby increase the efficiency of delivery of a therapeutic agent(s). In certain aspects, the disclosure provides methods of synthesizing the novel reversible zwitterionic lipids. In certain aspects, the disclosure provides mixed lipid particle compositions and formulations including the novel ionizable lipid(s) disclosed herein, as well as associated methods for delivery of lipid particle-associated molecular cargoes to the cells of a subject. In certain aspects, nucleic-acid lipid nanoparticles are provided that preferentially localize to and deliver associated nucleic acid cargoes to the liver, lung, skin, tumor or other tissue of a subject, with delivery optionally occurring to various types of tissue and/or population(s) of cells within a tissue of a subject.

In one aspect, the disclosure provides a pharmaceutical composition that includes a reversible zwitterionic lipid of Formula I having the following structure:

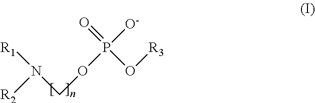

or a salt or isomer thereof, wherein
R₁ and R₂ are either the same or different and are independently C₇-C₂₂ alkyl, C₇-C₂₂ alkenyl, or C₇-C₂₂ alkynyl, optionally R₁, R₂, or R₁ and R₂ are an optionally substituted heterocycle or R₁ and R₂ may join to form an optionally substituted heterocycle;
R₃ is optionally substituted C₃-C₂₂ alkyl, C₃-C₂₂ alkenyl, or C₃-C₂₂ alkynyl; and
n is 3, 4, 5, 6, 7, 8, 9, 10, 11, 12, 13, 14, 15, 16, 17, 18, 19, 20, 21, or 22.

In some embodiments, R₁ and R₂ are the same.

In some embodiments, R₁ or R₂ are independently selected from the group consisting of C₇-C₁₈ alkyl, C₇-C₁₈ alkenyl, and C₇-C₁₈ alkynyl, and R₃ is optionally substituted C₇-C₁₈ alkyl, C₇-C₁₈ alkenyl, or C₇-C₁₈ alkynyl, optionally wherein R₁ and R₂ are independently selected from the group of C₇-C₁₈ alkyl, C₇-C₁₈ alkenyl, or C₇-C₁₈ alkynyl and R₃ is optionally substituted C₇-C₁₈ alkyl, C₇-C₁₈ alkenyl, or C₇-C₁₈ alkynyl. In some embodiments, n is 3 or 4.

In some embodiments, R₁ or R₂ are independently selected from the group consisting of C₇-C₁₂ alkyl, C₇-C₁₂ alkenyl, and C₇-C₁₂ alkynyl, and R₃ is optionally substituted C₇-C₁₂ alkyl, C₇-C₁₂alkenyl, or C₇-C₁₂ alkynyl, and n is 2, 3, 4, 5, 6, 7, or 8, optionally wherein R₁ and R₂ are independently selected from the group consisting of C₇-C₁₂ alkyl, C₇-C₁₂ alkenyl, and C₇-C₁₂ alkynyl and R₃ is optionally substituted C₇-C₁₂ alkyl, C₇-C₁₂ alkenyl, or C₇-C₁₂ alkynyl and n is 2, 3, 4, 5, 6, 7, or 8. In some embodiments, n is 3 or 4.

In some embodiments, R₁ is selected from the group consisting of C₇-C₁₀ alkyl, C₇-C₁₀ alkenyl, and C₇-C₁₀ alkynyl, R₂ is the same as R₁, and R₃ is optionally substituted C₇-C₁₂ alkyl, C₇-C₁₂ alkenyl, or C₇-C₁₂ alkynyl and n is 3, 4, 5, or 6. In some embodiments, n is 3 or 4.

In some embodiments, R₁ and R₂ are independently C₈-C₁₂ hydrocarbon, R₃ is optionally substituted C₈-C₁₂ hydrocarbon, and n is 3 or 4.

In some embodiments, R₁ is C₈-C₁₂ hydrocarbon, R₂ is the same as R₁, R₃ is optionally substituted C₈-C₁₂ hydrocarbon, and n is 3 or 4.

In some embodiments, R₁, R₂, and R₃ are independently an alkyl selected from the group consisting of heptane, octane, nonane, decane, undecane, and dodecane.

In some embodiments, one or more of R₁, R₂, and R₃ are independently an alkenyl selected from the group consisting of hept-1-ene, hept-2-ene, hept-3-ene, oct-1-ene, oct-2-ene, oct-3-ene, oct-4-ene, non-1-ene, non-2-ene, non-3-ene, non-4-ene, non-5-ene, dec-1-ene, dec-2-ene, dec-3-ene, dec-4-ene, dec-5-ene, dec-6-ene, undec-1-ene, undec-2-ene, undec-3-ene, undec-4-ene, undec-5-ene, undec-6-ene, undec-7-ene, dodec-1-ene, dodec-2-ene, dodec-3-ene, dodec-4-ene, dodec-5-ene, dodec-6-ene, and dodec-8-ene.

In some embodiments, one or more of R₁, R₂, and R₃ are independently an alkynyl selected from the group consisting of hept-1-yne, hept-2-yne, hept-3-yne, oct-1-yne, oct-2-yne, oct-3-yne, oct-4-yne, non-1-yne, non-2-yne, non-3-yne, non-4-yne, non-5-yne, dec-1-yne, dec-2-yne, dec-3-yne, dec-4-yne, dec-5-yne, dec-6-yne, undec-1-yne, undec-2-yne, undec-3-yne, undec-4-yne, undec-5-yne, undec-6-yne, undec-7-yne, dodec-1-yne, dodec-2-yne, dodec-3-yne, dodec-4-yne, dodec-5-yne, dodec-6-yne, and dodec-8-yne.

In one aspect, the disclosure provides a pharmaceutical composition including a reversible zwitterionic lipid selected from the group consisting of:

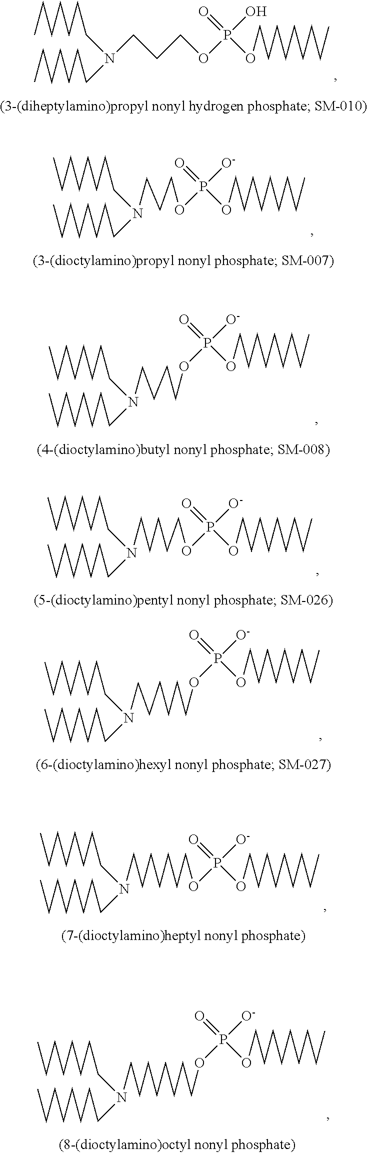

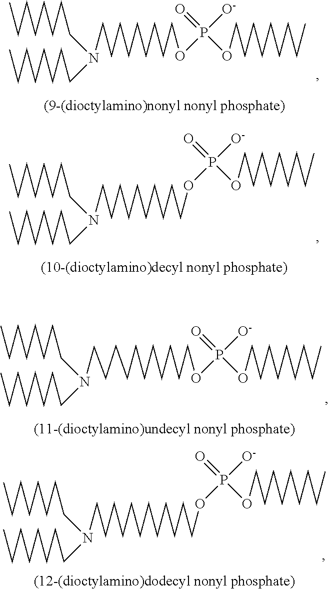

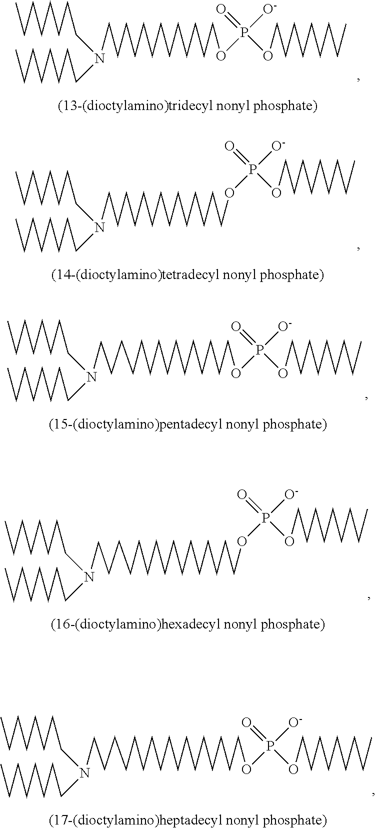

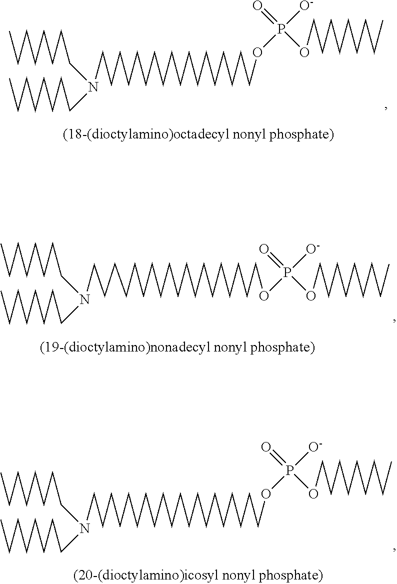

and salts and isomers thereof.

In one aspect, the disclosure provides a pharmaceutical composition including a reversible zwitterionic lipid selected from the group consisting of:

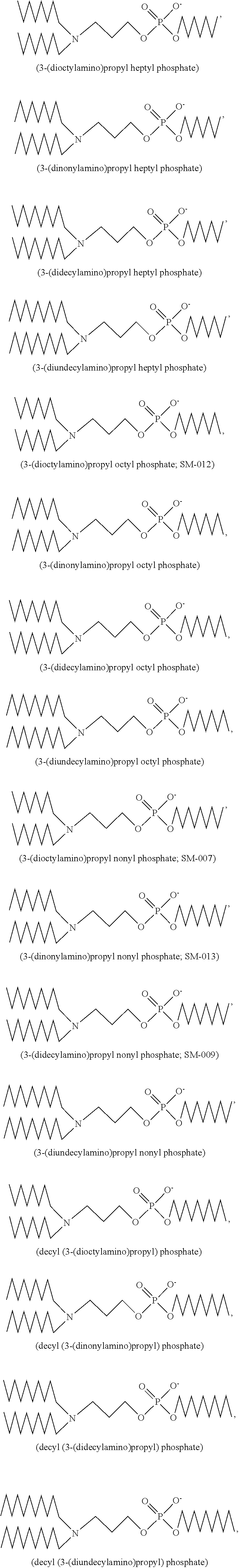

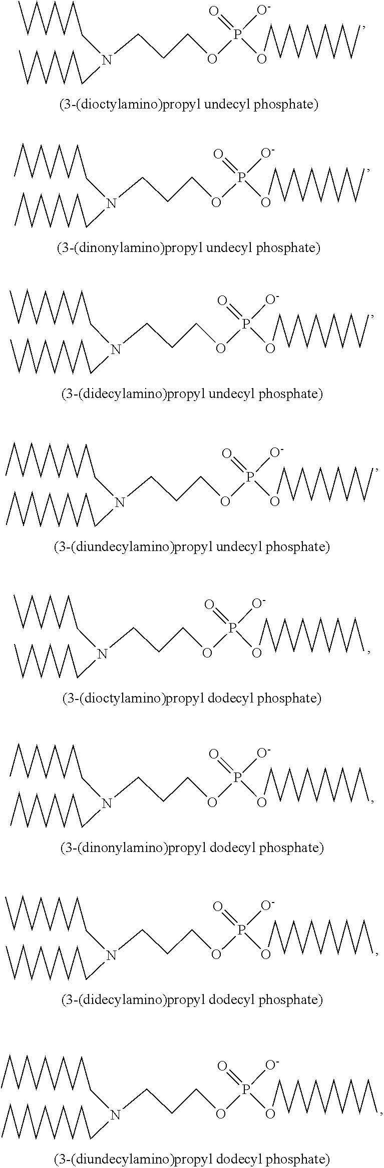

and salts and isomers thereof.

In one aspect, the disclosure provides a pharmaceutical composition including a reversible zwitterionic lipid selected from the group consisting of:

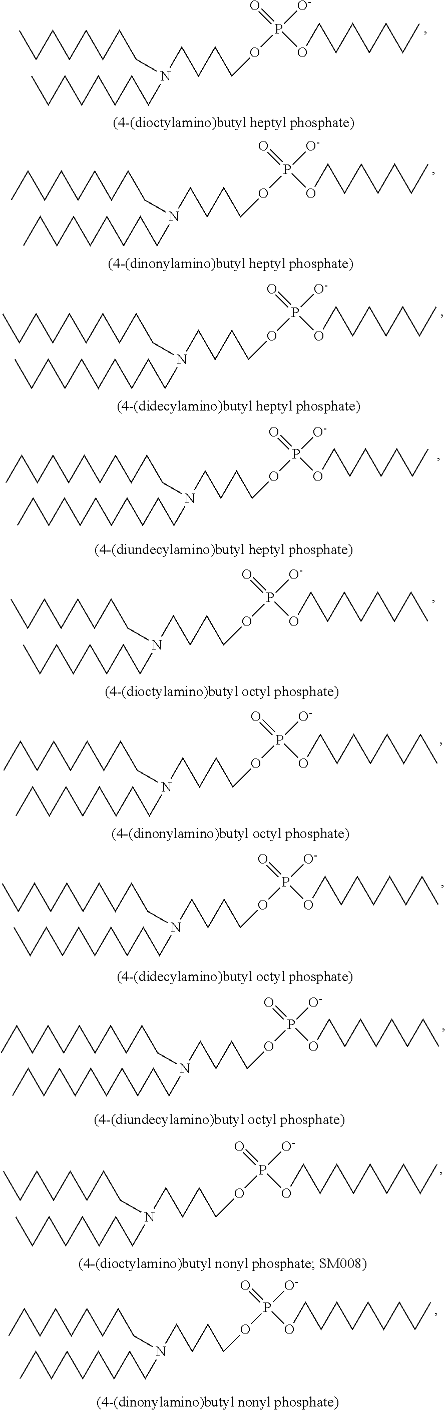

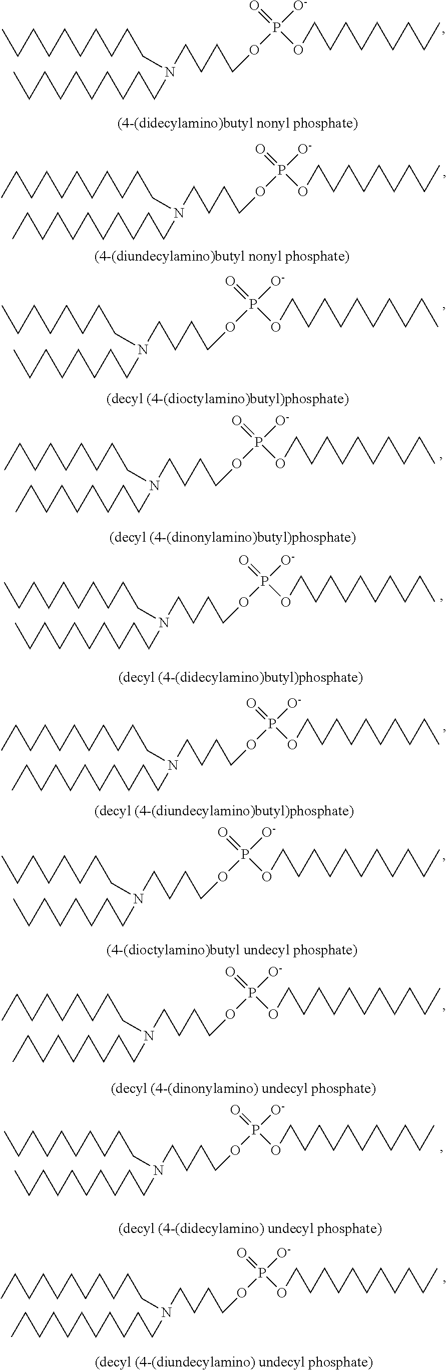

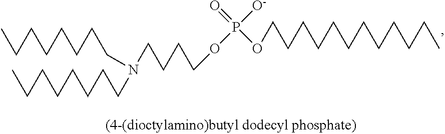 [SMILES: CCCCCCCCCCCCOP(=O)([O-])OCCCCN(CCCCCCCC)CCCCCCCC]

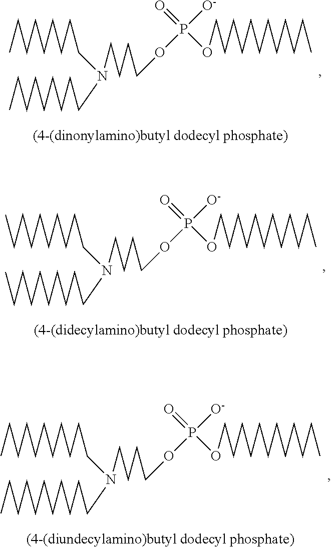

and salts and isomers thereof.

In one aspect, the disclosure provides a pharmaceutical composition including a reversible zwitterionic lipid selected from the group consisting of:

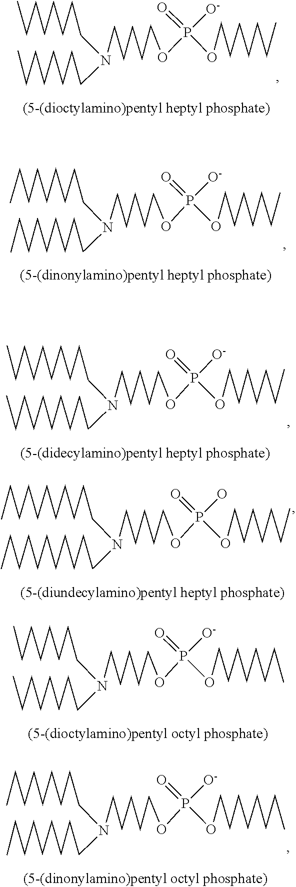

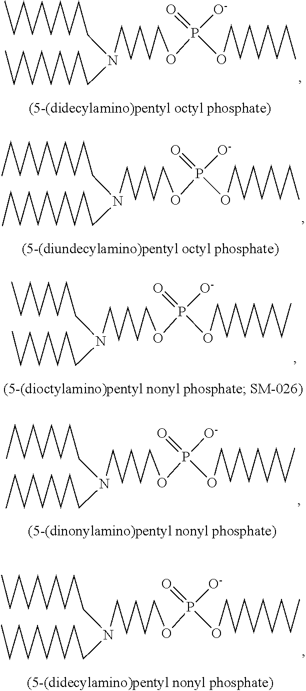

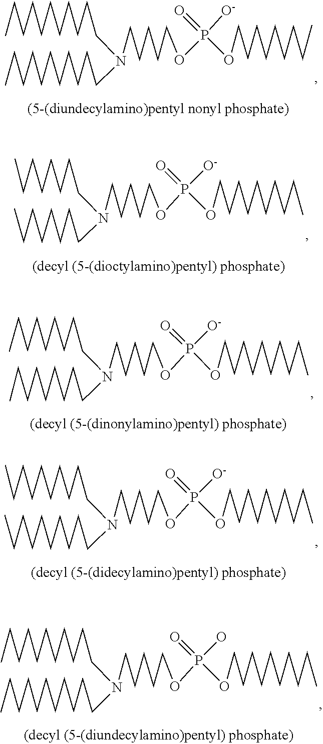

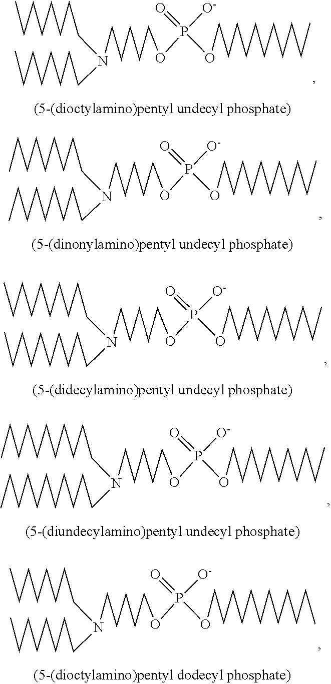

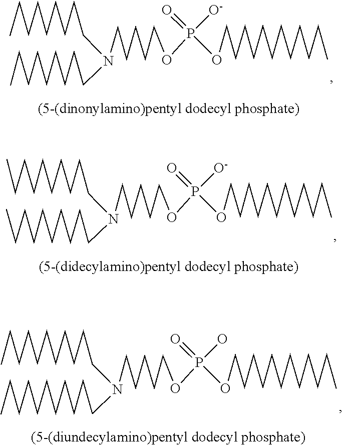

and salts and isomers thereof.

In one aspect, the disclosure provides a pharmaceutical composition including a reversible zwitterionic lipid selected from the group consisting of:

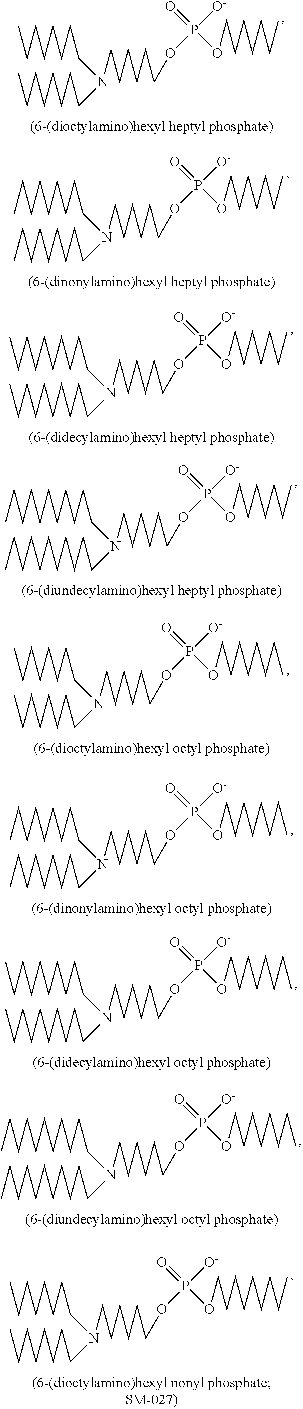

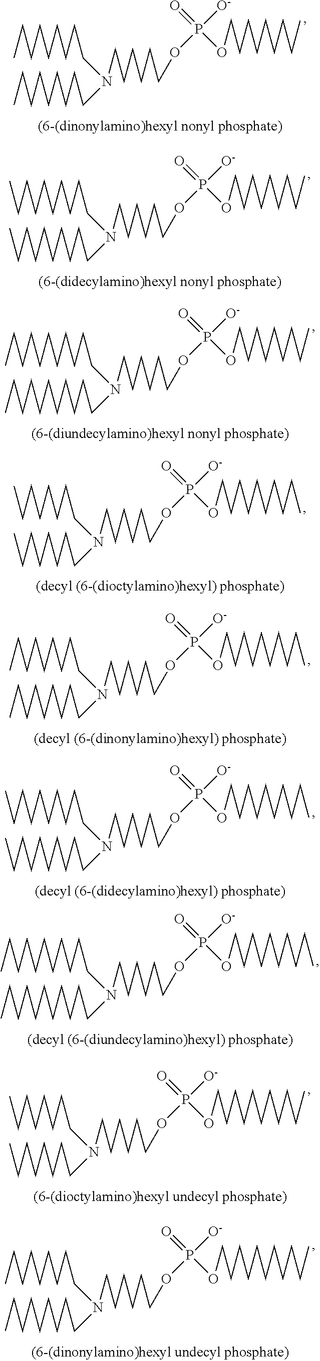

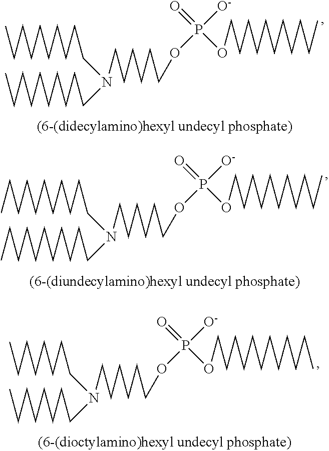

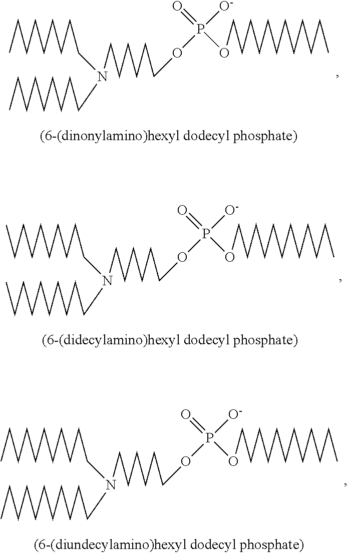

and salts and isomers thereof.

In one aspect, the disclosure provides a pharmaceutical composition including a reversible zwitterionic lipid selected from the group consisting of:

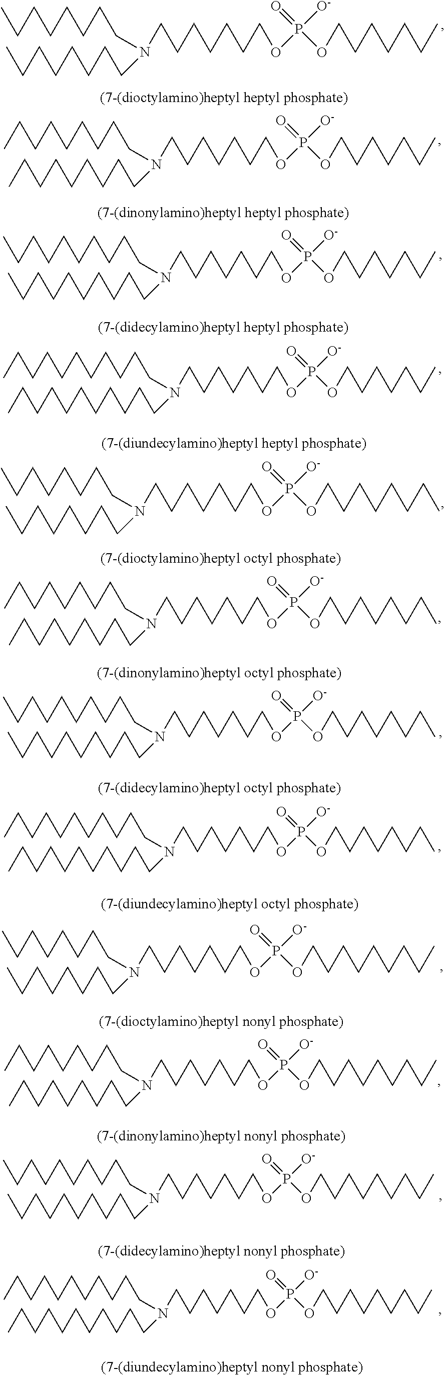

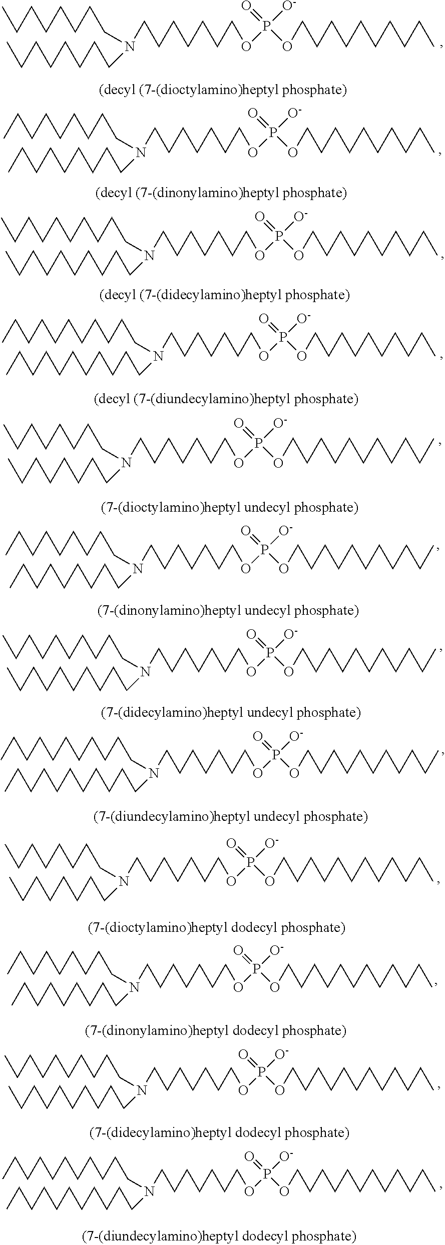

and salts and isomers thereof.

In one aspect, the disclosure provides a pharmaceutical composition including a reversible zwitterionic lipid selected from the group consisting of:

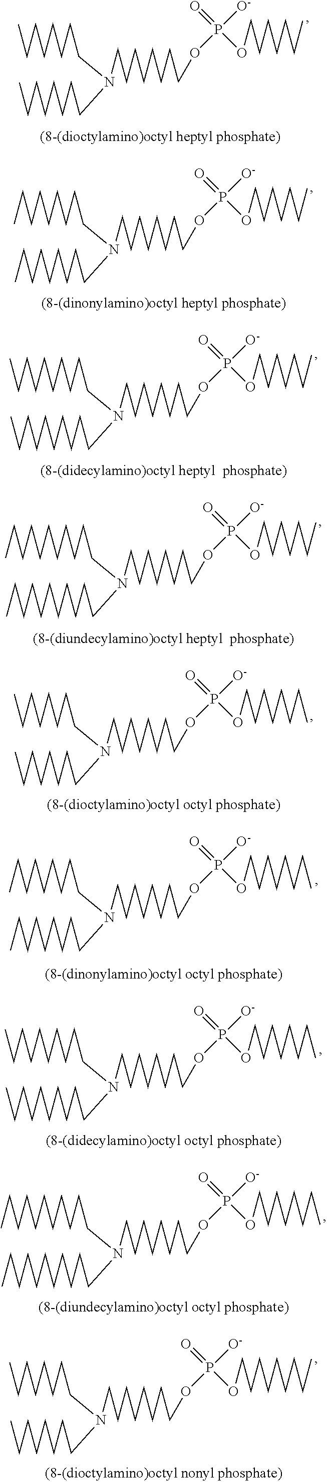

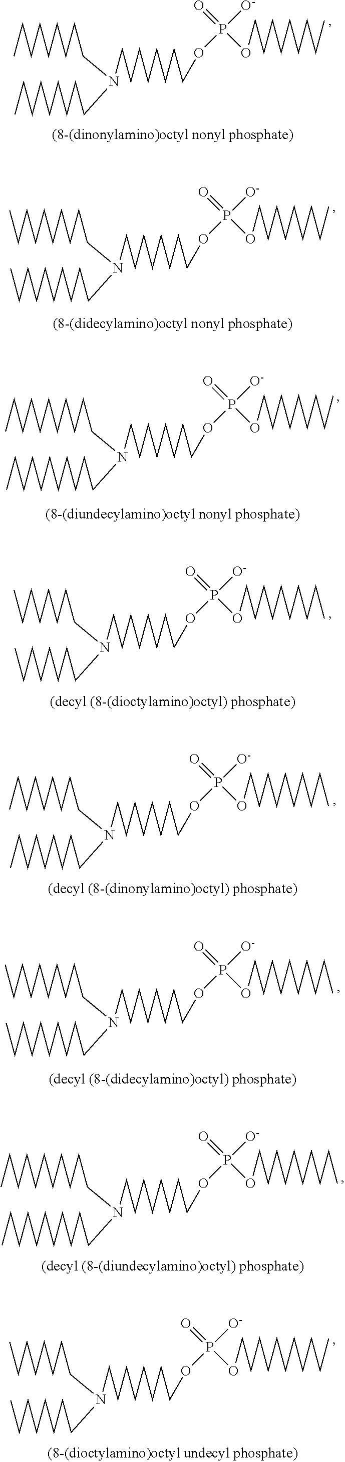

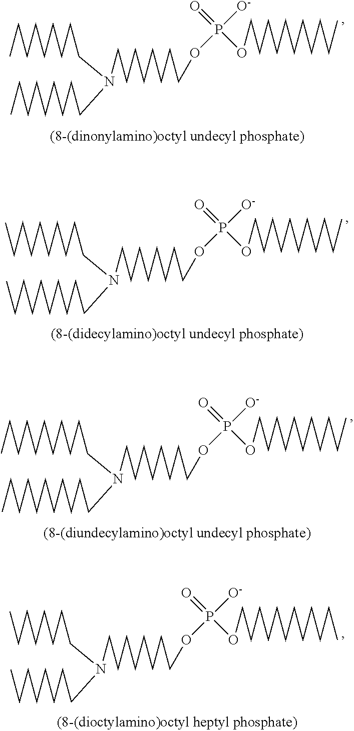

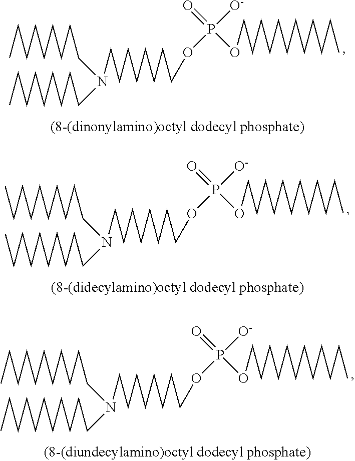

and salts and isomers thereof.

In one aspect, the disclosure provides a pharmaceutical composition including a reversible zwitterionic lipid selected from the group consisting of:

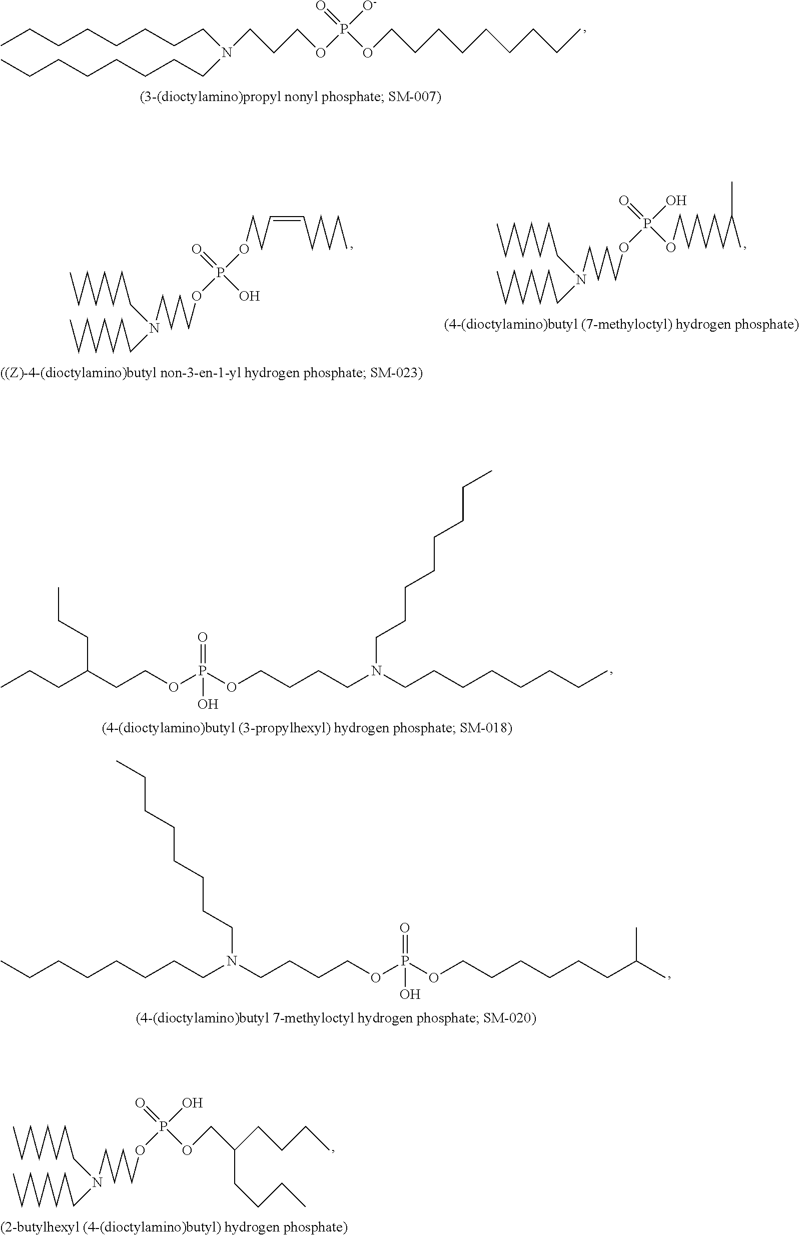

and salts and isomers thereof.

In one aspect, the disclosure provides a reversible zwitterionic lipid selected from the group consisting of:

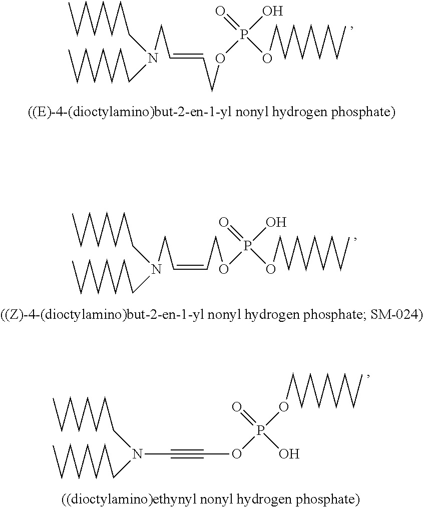

and salts and isomers thereof.

In another aspect, the disclosure provides a lipid particle including a reversible zwitterionic lipid selected from the group consisting of:

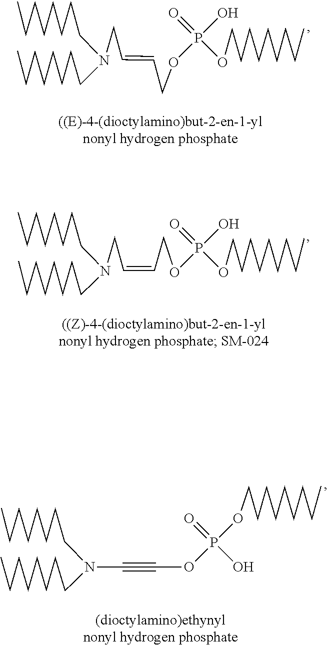

and salts and isomers thereof.

In some embodiments, the lipid particle further includes a therapeutic agent. In some embodiments, the therapeutic agent is a nucleic acid.

In one aspect, the disclosure provides a pharmaceutical composition comprising any of the above-referenced lipid particles and a pharmaceutically acceptable excipient, carrier, or diluent.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

As used herein, the term “alkyl” refers to a straight-chain or branched saturated hydrocarbon group having from 1 to 22 carbon atoms (“C₁₋₂₂ alkyl”). In some embodiments, an alkyl group may have 3 to 22 carbon atoms (“C₃₋₂₂ alkyl”) and/or 7 to 22 carbon atoms (“C₇₋₂₂ alkyl”). In some embodiments, an alkyl group may have 7 to 18 carbon atoms (“C₇₋₁₈ alkyl”) and/or 7 to 12 carbon atoms (“C₇₋₁₂ alkyl”). In some embodiments, an alkyl group has 7 to 8 carbon atoms (“C₇₋₈ alkyl”). In some embodiments, an alkyl group has 7 to 9 carbon atoms (“C₇₋₉ alkyl”). In some embodiments, an alkyl group may have 7 to 10 carbon atoms (“C₇₋₁₀ alkyl”). In some embodiments, an alkyl group has 7 to 11 carbon atoms (“C₇₋₁₁ alkyl”). In some embodiments, an alkyl group may have 8 to 12 carbon atoms (“C₈₋₁₂ alkyl”). In some embodiments, an alkyl group has 9 to 12 carbon atoms (“C₉₋₁₂ alkyl”). In some embodiments, an alkyl group has 10 to 12 carbon atoms (“C₁₀₋₁₂ alkyl”). In some embodiments, an alkyl group has 11 to 12 carbon atoms (“C₁₁₋₁₂ alkyl”). Additional examples of alkyl groups include n-heptyl (C₇), n-octyl (C₈), n-nonyl (C₉), n-decyl (C₁₀), n-undecyl (C₁₁), n-dodecyl (C₁₂), and the like.

An “alkyl” group as used herein may be unsubstituted or optionally substituted. Unless otherwise specified, each instance of an alkyl group is independently optionally substituted, i.e., unsubstituted (an “unsubstituted alkyl”) or substituted (a “substituted alkyl”) with one or more substituents. Suitable substituent groups may include, but are not limited to, hydroxyl, nitro, amino (e.g., —NH2 or dialkyl amino), imino, cyano, halo (e.g., F, Cl, Br, I, and the like), haloalkyl (e.g., —CCl₃, —CF₃, and the like), thio, sulfonyl, thioamido, amidino, imidino, oxo, oxamidino, methoxamidino, imidino, guanidino, sulfonamido, carboxyl, formyl, alkyl, alkoxy, alkoxy-alkyl, alkylcarbonyl, alkylcarbonyloxy (e.g., —OCOR), aminocarbonyl, arylcarbonyl, aralkylcarbonyl, carbonylamino, heteroarylcarbonyl, heteroaralkyl-carbonyl, alkylthio, aminoalkyl, cyanoalkyl, carbamoyl (e.g., —NHCOOR— or —OCONHR—), urea (e.g., —NHCONHR—), cycloalkyl, aryl, heteroaryl, heteroalicyclic, hydroxy, alkoxy, aryloxy, mercapto, alkylthio, arylthio, cyano, halo, carbonyl, (═O), thiocarbonyl, O-carbamyl, N-carbamyl, O-thiocarbamyl, N-thiocarbamyl, C-amido, N-amido, C-carboxy, O-carboxy, nitro, amino, heterocycle, —CN, and the like. An “alkyl” as used herein may be combined with other groups, such as those provided above, to form a functionalized alkyl.

An “alkyl” group, as defined herein, may further comprise 1 or more (e.g., 1, 2, 3, 4, etc.) heteroatoms (e.g., a “heteroalkyl” such as, e.g., oxygen, sulfur, nitrogen, boron, silicon, phosphorus, and the like) within the parent chain, wherein the one or more heteroatoms are inserted between adjacent carbon atoms within the parent carbon chain and/or one or more heteroatoms are inserted between a carbon atom and the parent molecule, i.e., between the point of attachment. In certain embodiments, a heteroalkyl group refers to a saturated group having from 1 to 22 carbon atoms and 1, 2, 3, 4, etc. heteroatoms (“heteroC₁₋₂₂ alkyl”). In some embodiments, a heteroalkyl group refers to a saturated group having from 3 to 22 carbon atoms and/or 7 to 22 carbon atoms and 1, 2, 3, 4, etc. heteroatoms (“heteroC₃₋₂₂ alkyl” and/or “hetero C₇₋₂₂ alkyl”). In some embodiments, a heteroalkyl group may have 7 to 18 carbon atoms and/or 7 to 12 carbon atoms and 1, 2, 3, 4, etc. heteroatoms (“heteroC₇₋₁₈ alkyl” and/or “hetero C₇₋₁₂ alkyl”). In some embodiments, a heteroalkyl group may have 7 to 8 carbon atoms and 1, 2, 3, 4, etc. heteroatoms (“heteroC₇₋₈ alkyl”). In some embodiments, a heteroalkyl group may have 7 to 9 carbon atoms and 1, 2, 3, 4, etc. heteroatoms (“heteroC₇₋₉ alkyl”). In some embodiments, a heteroalkyl group has 7 to 10 carbon atoms and 1, 2, 3, 4, etc. heteroatoms (“heteroC₇₋₁₀ alkyl”). In some embodiments, a heteroalkyl group has 7 to 11 carbon atoms and 1, 2, 3, 4, etc. heteroatoms (“heteroC₇₋₁₁ alkyl”). In some embodiments, a heteroalkyl group has 8 to 12 carbon atoms and 1, 2, 3, 4, etc. heteroatoms (“heteroC₈₋₁₂ alkyl”). In some embodiments, a heteroalkyl group has 9 to 12 carbon atoms and 1, 2, 3, 4, etc. heteroatoms (“heteroC₉₋₁₂ alkyl”). In some embodiments, a heteroalkyl group has 10 to 12 carbon atoms and 1, 2, 3, 4, etc. heteroatoms (“heteroC₁₀₋₁₂ alkyl”). In some embodiments, a heteroalkyl group has 11 to 12 carbon atoms and 1, 2, 3, 4, etc. heteroatoms (“heteroC₁₁₋₁₂ alkyl”).

As used herein, the term “alkenyl” includes a chain of carbon atoms, which is optionally branched, having from 2 to 22 carbon atoms and including at least one double bond (e.g., 1, 2, 3, 4, etc. carbon-carbon double bonds) (“C₂₋₂₂ alkenyl”). In some embodiments, an alkenyl group may have 3 to 22 carbon atoms (“C₃₋₂₂ alkenyl”) and/or 7 to 22 carbon atoms (“C₇₋₂₂ alkenyl”). In some embodiments, an alkenyl group may have 7 to 18 carbon atoms (“C₇₋₁₈ alkenyl”) and/or 7 to 12 carbon atoms (“C₇₋₁₂ alkenyl”). In some embodiments, an alkenyl group has 7 to 8 carbon atoms (“C₇₋₈ alkenyl”). In some embodiments, an alkenyl group has 7 to 9 carbon atoms (“C₇₋₉ alkenyl”). In some embodiments, an alkenyl group may have 7 to 10 carbon atoms (“C₇₋₁₀ alkenyl”). In some embodiments, an alkenyl group has 7 to 11 carbon atoms (“C₇₋₁₁ alkenyl”). In some embodiments, an alkenyl group may have 8 to 12 carbon atoms (“C₈₋₁₂ alkenyl”). In some embodiments, an alkenyl group has 9 to 12 carbon atoms (“C₉₋₁₂ alkenyl”). In some embodiments, an alkenyl group has 10 to 12 carbon atoms (“C₁₀₋₁₂ alkenyl”). In some embodiments, an alkenyl group has 11 to 12 carbon atoms (“C₁₁₋₁₂ alkenyl”). Additional examples of alkenyl groups include n-heptyl (C₇), n-octyl (C₈), n-nonyl (C₉), n-decyl (C₁₀), n-undecyl (C₁₁), n-dodecyl (C₁₂), and the like. The one or more carbon-carbon double bonds may be internal (e.g., 2-butenyl) or terminal (e.g., 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

Unless otherwise specified, each instance of an alkenyl group is independently optionally substituted, i.e., unsubstituted (an “unsubstituted alkenyl”) or substituted (a “substituted alkenyl”) with one or more substituents e.g., from 1 to 5 substituents, 1 to 3 substituents, or 1 substituent. In certain embodiments, the alkenyl group is unsubstituted C₃₋₂₂ alkenyl. In certain embodiments, the alkenyl group is substituted C₃₋₂₂ alkenyl. Exemplary substituents are listed above with respect to “alkyl” and may be used here with respect to “alkenyl” as well.

The term “heteroalkenyl,” as used herein, refers to an alkenyl group, as defined above, which further comprises one or more (e.g., 1, 2, 3, 4, etc.) heteroatoms (e.g., oxygen, sulfur, nitrogen, boron, silicon, phosphorus, and the like), wherein the one or more heteroatoms is inserted between adjacent carbon atoms within the parent carbon chain and/or one or more heteroatoms are inserted between a carbon atom and the parent molecule, i.e., between the point of attachment. In some embodiments, a heteroalkenyl group refers to an unsaturated group having 2 to 22 carbon atoms and 1, 2, 3, 4, etc. heteroatoms (“heteroC₂₋₂₂ alkenyl”). In some embodiments, a heteroalkenyl group refers to an unsaturated group having from 7 to 18 carbon atoms and/or 7 to 12 carbon atoms and 1, 2, 3, 4, etc. heteroatoms (“heteroC₇₋₁₈ alkenyl” or “hetero C₇₋₁₂ alkenyl”). In some embodiments, a heteroalkenyl group may have 7 to 8 carbon atoms and 1, 2, 3, 4, etc. heteroatoms (“heteroC₇₋₈ alkenyl”). In some embodiments, a heteroalkenyl group may have 7 to 9 carbon atoms and 1, 2, 3, 4, etc. heteroatoms (“heteroC₇₋₉ alkenyl”). In some embodiments, a heteroalkenyl group has 7 to 10 carbon atoms and 1, 2, 3, 4, etc. heteroatoms (“heteroC₇₋₁₀ alkenyl”). In some embodiments, a heteroalkenyl group has 7 to 11 carbon atoms and 1, 2, 3, 4, etc. heteroatoms (“heteroC₇₋₁₁ alkenyl”). In some embodiments, a heteroalkenyl group has 8 to 12 carbon atoms and 1, 2, 3, 4, etc. heteroatoms (“heteroC₈₋₁₂ alkenyl”). In some embodiments, a heteroalkenyl group has 9 to 12 carbon atoms and 1, 2, 3, 4, etc. heteroatoms (“heteroC₉₋₁₂ alkenyl”). In some embodiments, a heteroalkenyl group has 10 to 12 carbon atoms and 1, 2, 3, 4, etc. heteroatoms (“heteroC₁₀₋₁₂ alkenyl”). In some embodiments, a heteroalkenyl group has 11 to 12 carbon atoms and 1, 2, 3, 4, etc. heteroatoms (“heteroC₁₁₋₁₂ alkenyl”). Additional examples of alkenyl groups include n-heptyl (C₇), n-octyl (C₈), n-nonyl (C₉), n-decyl (C₁₀), n-undecyl (Cu), n-dodecyl (C₁₂), and the like. The one or more carbon-carbon double bonds can be internal (such as in 2-butenyl) or terminal (such as in 1-butenyl). Examples of alkenyl include heptenyl (C₇), octenyl (C₈), octatrienyl (C₈), and the like.

As used herein, the term “alkynyl” includes a chain of carbon atoms, which is optionally branched, and contains from 2 to 22 carbon atoms (“C₂₋₂₂ alkynyl”), including at least one carbon-carbon triple bond (i.e., C≡C). In some embodiments, an alkynyl group may have 3 to 22 carbon atoms (“C₃₋₂₂ alkynyl”) and/or 7 to 22 carbon atoms (“C₇₋₂₂ alkynyl”). In some embodiments, an alkynyl group may have 7 to 18 carbon atoms (“C₇₋₁₈ alkynyl”) and/or 7 to 12 carbon atoms (“C₇₋₁₂ alkynyl”). In some embodiments, an alkynyl group has 7 to 8 carbon atoms (“C₇₋₈ alkynyl”). In some embodiments, an alkynyl group has 7 to 9 carbon atoms (“C₇₋₉ alkynyl”). In some embodiments, an alkynyl group may have 7 to 10 carbon atoms (“C₇₋₁₀ alkynyl”). In some embodiments, an alkynyl group has 7 to 11 carbon atoms (“C₇₋₁₁ alkynyl”). In some embodiments, an alkynyl group may have 8 to 12 carbon atoms (“C₈₋₁₂ alkynyl”). In some embodiments, an alkynyl group has 9 to 12 carbon atoms (“C₉₋₁₂ alkynyl”). In some embodiments, an alkynyl group has 10 to 12 carbon atoms (“C₁₀₋₁₂ alkynyl”). In some embodiments, an alkynyl group has 11 to 12 carbon atoms (“C₁₁₋₁₂ alkynyl”).

Alkynyl may be unsubstituted or substituted as described above for “alkyl” or as described in the various embodiments provided herein. Illustrative alkynyl groups include, but are not limited to, ethynyl, 1-propynyl, 2-propynyl, 1-, 2-, or 3-butynyl, and the like.

The term “heteroalkynyl,” as used herein, refers to an alkynyl group, as defined above, which further comprises one or more (e.g., 1, 2, 3, 4, etc.) heteroatoms (e.g., oxygen, sulfur, nitrogen, boron, silicon, phosphorus, and the like), wherein the one or more heteroatoms are inserted between adjacent carbon atoms within the parent carbon chain and/or one or more heteroatoms are inserted between a carbon atom and the parent molecule, i.e., between the point of attachment. In some embodiments, a heteroalkynyl group refers to an unsaturated group having 2 to 22 carbon atoms and 1, 2, 3, 4, etc. heteroatoms (“heteroC₂₋₂₂ alkynyl”). In some embodiments, a heteroalkynyl group refers to an unsaturated group having from 7 to 18 carbon atoms and/or 7 to 12 carbon atoms and 1, 2, 3, 4, etc. heteroatoms (“heteroC₇₋₁₈ alkynyl” or “hetero C₇₋₁₂ alkynyl”). In some embodiments, a heteroalkynyl group may have 7 to 8 carbon atoms and 1, 2, 3, 4, etc. heteroatoms (“heteroC₇₋₈ alkynyl”). In some embodiments, a heteroalkynyl group may have 7 to 9 carbon atoms and 1, 2, 3, 4, etc. heteroatoms (“heteroC₇₋₉ alkynyl”). In some embodiments, a heteroalkynyl group has 7 to 10 carbon atoms and 1, 2, 3, 4, etc. heteroatoms (“heteroC₇₋₁₀ alkynyl”). In some embodiments, a heteroalkynyl group has 7 to 11 carbon atoms and 1, 2, 3, 4, etc. heteroatoms (“heteroC₇₋₁₁ alkynyl”). In some embodiments, a heteroalkynyl group has 8 to 12 carbon atoms and 1, 2, 3, 4, etc. heteroatoms (“heteroC₈₋₁₂ alkynyl”). In some embodiments, a heteroalkynyl group has 9 to 12 carbon atoms and 1, 2, 3, 4, etc. heteroatoms (“heteroC₉₋₁₂ alkynyl”). In some embodiments, a heteroalkynyl group has 10 to 12 carbon atoms and 1, 2, 3, 4, etc. heteroatoms (“heteroC₁₀₋₁₂ alkynyl”). In some embodiments, a heteroalkynyl group has 11 to 12 carbon atoms and 1, 2, 3, 4, etc. heteroatoms (“heteroC₁₁₋₁₂ alkynyl”).

As used herein, “carbocyclyl” or “carbocyclic” refers to a radical of a non-aromatic cyclic hydrocarbon group having from 3 to 8 ring carbon atoms (“C₃₋₈ carbocyclyl”) and zero heteroatoms in the non-aromatic ring system. In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 8 ring carbon atoms (“C₅₋₈ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₀ carbocyclyl. In certain embodiments, the carbocyclyl group is a substituted C₃₋₁₀ carbocyclyl.

In some embodiments, “carbocyclyl” or “carbocyclic” is referred to as a “cycloalkyl”, i.e., a monocyclic, saturated carbocyclyl group having from 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 8 ring carbon atoms (“C₅₋₈ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₈ cycloalkyl. In certain embodiments, the cycloalkyl group is a substituted C₃₋₈ cycloalkyl.

The term “heterocycle” or “heterocyclyl” refers to a saturated or an unsaturated aromatic or non-aromatic group having from 1 to 8 annular carbon atoms and from 1 to 4 annular heteroatoms, such as nitrogen, oxygen, sulfur, boron, phosphorus, silicon, and the like, wherein the nitrogen and sulfur atoms are optionally oxidized, and the nitrogen atom(s) are optionally quaternized. A heterocycle group may have a single ring or multiple condensed rings. A heterocycle comprising more than one ring may be fused, spiro or bridged, or any combination thereof. In fused ring systems, one or more of the fused rings can be aryl or heteroaryl. Examples of heterocycle groups include, but are not limited to, dihydropyranyl, thiazolinyl, thiazolidinyl, tetrahydrothiophenyl, 2,3-dihydrobenzo[b]thiophen-2-yl, 4-amino-2-oxopyrimidin-1(2H)-yl, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, N-oxides thereof, and the like. A “heterocycle” as disclosed herein may be optionally substituted with one or more substituents, including e.g., but not limited to, hydroxyl, nitro, amino (e.g., —NH2 or dialkyl amino), imino, cyano, halo (e.g., F, Cl, Br, I, and the like), haloalkyl (e.g., —CCl₃, —CF₃, and the like), thio, sulfonyl, thioamido, amidino, imidino, oxo, oxamidino, methoxamidino, imidino, guanidino, sulfonamido, carboxyl, formyl, alkyl, alkoxy, alkoxy-alkyl, alkylcarbonyl, alkylcarbonyloxy (e.g., —OCOR), aminocarbonyl, arylcarbonyl, aralkylcarbonyl, carbonylamino, heteroarylcarbonyl, heteroaralkyl-carbonyl, alkylthio, aminoalkyl, cyanoalkyl, carbamoyl (e.g., —NHCOOR— or —OCONHR—), urea (e.g., —NHCONHR—), cycloalkyl, aryl, heteroaryl, heteroalicyclic, hydroxy, alkoxy, aryloxy, mercapto, alkylthio, arylthio, cyano, halo, carbonyl, (═O), thiocarbonyl, O-carbamyl, N-carbamyl, O-thiocarbamyl, N-thiocarbamyl, C-amido, N-amido, C-carboxy, O-carboxy, nitro, amino, heterocycle, —CN, and the like. For example and without limitation, additional optional substituents include fluorine, chlorine, bromine, and iodine atoms and CF₃, CN, OH, ═O, SH, ═S, NH₂, ═NH, N₃ and NO₂ groups. Optional substituents also include C₁-C₁₀ alkyl, C₁-C₁₀ heteroalkyl, C₁-C₁₀ alkenyl, C₁-C₁₀ heteroalkenyl, C₁-C₁₀ alkynyl, C₁-C₁₀ hetero alkynyl, and the like. Exemplary substituents are F, Cl, Br, OH, SH, ═O, NH2, amino, C₁₋₄ alkyl (e.g., methyl, ethyl, t-butyl), C₁₋₄ heteroalkyl cyclopropyl, SFs, NO, NO₂, NMe2, CONH₂, CH₂NMe2, NHSO₂Me, C(CH₃)₂CN, COMe, OMe, SMe, COOMe, COOEt, CH₂COOH, OCH₂COOH, COOH, SOMe, SO₂Me, cyclopropyl, SO₂NH₂, SO₂NHMe, SO₂CH₂CH₂OH, NHCH₂CH₂OH, CH₂CH₂OCH₃, SFs, SO₂NMe2, NO, NO₂, OCF₃, SO₂CF₃, CN or CF₃.

In heterocycle groups that contain one or more nitrogen atoms, the point of attachment can be a carbon or nitrogen atom, as valency permits. A heterocycle group can either be monocyclic (“monocyclic heterocycle”) or a fused, bridged or spiro ring system such as a bicyclic system (“bicyclic heterocycle”), and can be saturated or can be partially unsaturated. Heterocycle bicyclic ring systems can include one or more heteroatoms in one or both rings. “Heterocycle” also includes ring systems wherein the heterocycle ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocycle”) with one or more substituents. In certain embodiments, the heterocycle group is unsubstituted 3-8 membered heterocycle. In certain embodiments, the heterocycle group is substituted 3-8 membered heterocycle.

In some embodiments, a heterocycle group is a 3-8 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocycle”). In some embodiments, a heterocycle group is a 5-8 membered non-aromatic ring system having ring carbon atoms and 1-4 ring heteroatoms, wherein each heteroatom is independently selected from nitrogen, oxygen, and sulfur (“5-8 membered heterocycle”). In some embodiments, a heterocycle group is a 5-6 membered non-aromatic ring system having ring carbon atoms and 1-4 ring heteroatoms, wherein each heteroatom is independently selected from nitrogen, oxygen, and sulfur (“5-6 membered heterocycle”). In some embodiments, the 5-6 membered heterocycle has 1-3 ring heteroatoms selected from nitrogen, oxygen, and sulfur. In some embodiments, the 5-6 membered heterocycle has 1-2 ring heteroatoms selected from nitrogen, oxygen, and sulfur. In some embodiments, the 5-6 membered heterocycle has one ring heteroatom selected from nitrogen, oxygen, and sulfur.

As used herein, the expression “optionally substituted” means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Heteroatoms, such as nitrogen, may have substituents, such as any suitable substituent described herein which satisfies the valencies of the heteroatoms and results in the formation of a stable moiety.

The term “lipid” refers to a group of organic compounds that include, but are not limited to, esters of fatty acids and are characterized by being insoluble in water, but soluble in many organic solvents. They are usually divided into at least three classes: (1) “simple lipids” which include fats and oils as well as waxes; (2) “compound lipids” which include phospholipids and glycolipids; (3) “derived lipids” such as steroids.

As used herein, the term “reversible zwitterionic lipid” refers to any lipid species that includes a potentially anionic group, such as a phosphate group, a reversibly cationic group, such as an ionizable amine group, and at least one hydrophobic tail. The zwitterion constituted by the ionizable amine group and the phosphate group of these reversible zwitterionic lipids is reversible at different pHs. For example, at physiological pH (e.g., ˜7.4) the tertiary amine group will not be protonated; however, at acidic pH, the tertiary amine will be protonated to form a zwitterionic head. For example, and without limitation, a reversible zwitterionic lipid may have a primary, secondary, or tertiary amine as a head group, (e.g., an alkylamino or dialkylamino head group) and a phosphate group that are separated by a linker. In some embodiments, the reversible zwitterionic lipids comprise: an ionizable amine (e.g., pH-titratable) head group, a linker, and a phosphate group, where the ionizable amine head group includes two hydrocarbon chains (e.g., C₇-C₂₂ alkyl, alkenyl, or alkynyl) and the phosphate group includes one hydrocarbon chain (e.g., C₃-C₂₂ alkyl, alkenyl, or alkynyl), where each of the aforementioned hydrocarbon chains independently has 0 to 3 (e.g., 0, 1, 2, or 3) double bonds, and ether, ester, or ketal linkages between the ionizable amine head group and hydrocarbon chains.

As used herein, the term “cationic lipid” refers to any lipid species that carries a net positive charge at a selected pH such as, for example, physiological pH. A cationic lipid may have a head group that is always positively charged in aqueous solution (an “obligate cationic lipid”). For example and without limitation, an obligate cationic lipid may have a quaternary amine as a head group. Alternatively, a cationic lipid may have a head group that accepts a proton in solution such that the lipid exists predominantly as a cation below its pKa and predominantly as a neutral moiety above its pKa, e.g., it may have a pH-titratable amino head group (e.g., for an “ionizable lipid”, as defined infra). For example, and without limitation, an ionizable lipid may have a primary, secondary, or tertiary amine as a head group, (e.g., an alkylamino or dialkylamino head group). In some embodiments, the ionizable lipids comprise: a protonatable tertiary amine (e.g., pH-titratable) head group; C18 hydrocarbon chains e.g., alkyl, alkenyl, or alkynyl chains, wherein each hydrocarbon chain independently has 0 to 3 (e.g., 0, 1, 2, or 3) double bonds; and ether, ester, or ketal linkages between the head group and hydrocarbon chains.

Examples of obligate cationic lipids include, but are not limited to, Dimethyldioctadecylammonium, Bromide Salt (DDAB), N-(4-carboxybenzyl)-N,N-dimethyl-2,3-bis(oleoyloxy) propan-1-aminium (DOBAQ), 1,2-dioleoyl-3-trimethylammonium-propane or 18:1 TAP, a di-chain or gemini, cationic lipid (DOTAP), 1,2-di-O-octadecenyl-3-trimethylammonium propane, chloride salt (DOTMA), ethyl phosphatidylcholine (EPC), and trimethyl sphingosine.

A range of forms of the obligate cationic lipid EPC are commercially available. Ethyl phosphatidylcholine, 18:1 EPC (Cl Salt), also known as 1,2-dioleoyl-sn-glycero-3-ethylphosphocholine (chloride salt), has the following structure:

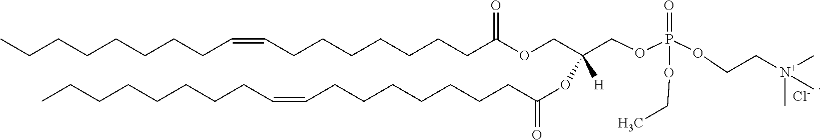 [SMILES: CCCCCCCC/C=C\CCCCCCCC(=O)OC[C@H](COP(=O)(OCC)OCC[N+](C)(C)C)OC(=O)CCCCCCC/C=C\CCCCCCCC ; [Cl-]]

18:0 EPC (Cl Salt), also known as 1,2-distearoyl-sn-glycero-3-ethylphospnocholine (chloride salt), has the following structure:

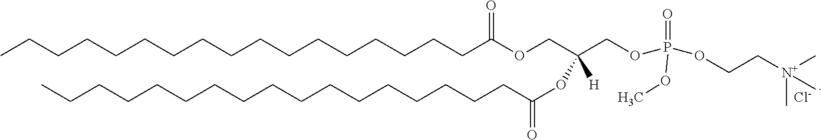 [SMILES: CCCCCCCCCCCCCCCCCC(=O)OC[C@H](COP(=O)(OC)OCC[N+](C)(C)C)OC(=O)CCCCCCCCCCCCCCCCC ; [Cl-]]

16:0 EPC (Cl Salt), also known as 1,2-dipalmitoyl-sn-glycero-3-ethylphosphocholine (chloride salt), has the following structure:

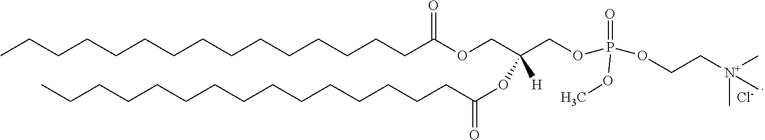 [SMILES: CCCCCCCCCCCCCCCC(=O)OC[C@H](COP(=O)(OC)OCC[N+](C)(C)C)OC(=O)CCCCCCCCCCCCCCC ; [Cl-]]

14:0 EPC (Cl Salt), also known as 1,2-dimyristoyl-sn-glycero-3-ethylphosphocholine (chloride salt), has the following structure:

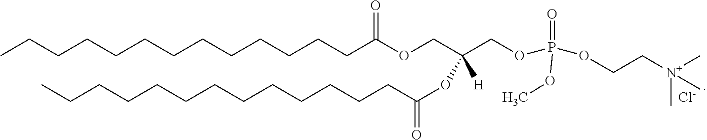 [SMILES: CCCCCCCCCCCCCC(=O)OC[C@H](COP(=O)(OC)OCC[N+](C)(C)C)OC(=O)CCCCCCCCCCCCC ; [Cl-]]

12:0 EPC (Cl Salt), also known as 1,2-dilauroyl-sn-glycero-3-ethylphosphocholine (chloride salt), has the following structure:

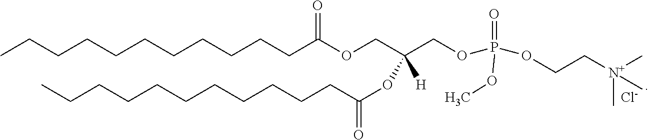 [SMILES: CCCCCCCCCCCC(=O)OC[C@H](COP(=O)(OC)OCC[N+](C)(C)C)OC(=O)CCCCCCCCCCC ; [Cl-]]

14:1 EPC (Tf Salt), also known as 1,2-dimyristoleoyl-sn-glycero-3-ethylphosphocholine (Tf salt), has the following structure:

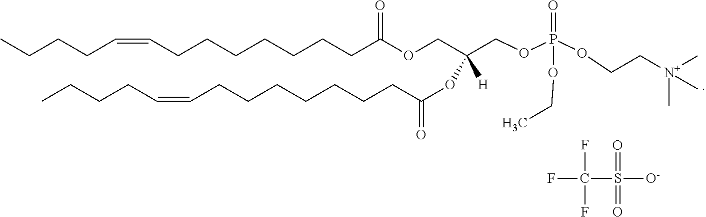 [SMILES: CCCC/C=C\CCCCCCCC(=O)OC[C@H](COP(=O)(OCC)OCC[N+](C)(C)C)OC(=O)CCCCCCC/C=C\CCCC ; O=S(=O)([O-])C(F)(F)F]

16:0-18:1 EPC (Cl Salt), also known as 1-palmitoyl-2-oleoyl-sn-glycero-3-ethylphosphocholine (chloride salt), has the following structure:

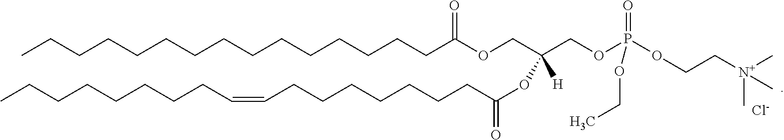 [SMILES: CCCCCCCC/C=C\CCCCCCCC(=O)O[C@H](COC(=O)CCCCCCCCCCCCCCC)COP(=O)(OCC)OCC[N+](C)(C)C ; [Cl-]]

18:1 EPC (Cl Salt), also known as 1,2-dioleoyl-sn-glycero-3-ethylphosphocholine (chloride salt), has the following structure:

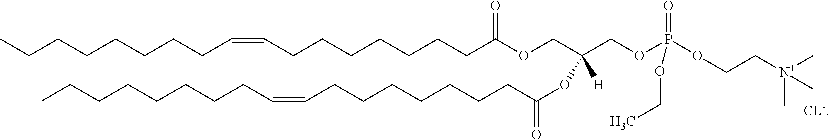

As used herein, the term “ionizable lipid” or “ionizable cationic lipid” refers to a lipid that becomes cationic (protonated) as the pH is lowered below the pKa of the ionizable group of the lipid but is progressively more neutral at higher pH values. When a component of a lipid-nucleic acid particle, at pH values below the pKa, the lipid is then able to associate with negatively charged polynucleic acids. Certain examples of such ionizable lipids include lipids and salts thereof having one, two, three, or more fatty acid or fatty hydrocarbon chains and a pH-titratable amino head group (e.g., an alkylamino or dialkylamino head group). Exemplary ionizable lipids include, without limitation, 1,2-Dioleoyl-3-dimethylammonium-propane (DODAP), 9-Heptadecanyl 8-{(2-hydroxyethyl)[6-oxo-6-(undecyloxy)hexyl]amino}octanoate (SM-102), disulfanediylbis(ethane-2,1-diyl)bis(piperidine-1,4-diyl)bis(ethane-2,1-diyl)bis(oxy)bis(2-oxoethane-2,1-diyl)bis(4,1-phenylene) dioleate (SS-OP), Dimethyl Sphingosine, 3-(N—(N′,N′-dimethylaminoethane)-carbamoyl)cholesterol (DC-Cholesterol), C12-200; N4-Cholesteryl-Spermine HCl Salt (GL67); N1-[2-((1S)-1-[(3-aminopropyl)amino]-4-[di(3-amino-propyl)amino]butylcarboxamido)ethyl]-3,4-di[oleyloxy]-benzamide (MVL5); 1,2-distearyloxy-N,N-dimethyl-3-aminopropane (DSDMA); 1,2-dioleyloxy-N,N-dimethyl-3-aminopropane (DODMA); 1,2-dilinoleyloxy-N,N-dimethyl-3-aminopropane (DLinDMA); 1,2-dilinolenyloxy-N,N-dimethyl-3-aminopropane (DLenDMA); 1,2-di-7-linolenyloxy-N,N-dimethylaminopropane (γ-DLenDMA); 1,2-dilinoleyloxy-keto-N,N-dimethyl-3-aminopropane (DLinK-DMA); 1,2-dilinoleyl-4-(2-dimethylaminoethyl)-[1,3]-dioxolane (DLinKC2-DMA) (also known as DLin-C2K-DMA, XTC2, and C2K); 2,2-dilinoleyl-4-(3-dimethylaminopropyl)[1,3]-dioxolane (DLin-K-C3-DMA); 2,2-dilinoleyl-4-(4-dimethylaminobutyl)[1,3]-dioxolane (DLin-K-C4-DMA); 1,2-dilinolenyloxy-4-(2-dimethylaminoethyl)-[1,3]-dioxolane (γ-DLen-C2K-DMA); 1,2-di-7-linolenyloxy-4-(2-dimethylaminoethyl)-[1,3]-dioxolane (γ-DLen-C2K-DMA); dilinoleylmethyl-3-dimethylaminopropionate (DLin-M-C2-DMA) (also known as MC2); (6Z,9Z,28Z,31Z)-heptatriaconta-6,9,28,31-tetraen-19-yl 4-(dimethylamino) butanoate (DLin-M-C3-DMA) (also known as MC3); 3-(dilinoleylmethoxy)-N,N-dimethylpropan-1-amine (DLin-MP-DMA) (also known as 1-B11); 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA); (2R) 2-({8-[(3β)-cholest-5-en-3-yloxy]octyl}oxy)-N,N-dimethyl-3-[(9Z,12Z)-octadeca-9,12-dien-1-yloxy]propan-1-amine (R-Octyl-CLinDMA); (2S) 2-({8-[(3β)-cholest-5-en-3-yloxy]octyl}oxy)-N,N-dimethyl-3-[(9Z,12Z)-octadeca-9,12-dien-1-yloxy]propan-1-amine (S-Octyl-CLinDMA); (2S)-1-{7-[(3β)-cholest-5-en-3-yloxy]heptyloxy}-3-[(4Z)-dec-4-en-1-yloxy]-N,N-dimethylpropan-2-amine; (2R)-1-{4-[(3P)-cholest-5-en-3-yloxy]butoxy}-3-[(4Z)-dec-4-en-1-yloxy]-N,N-dimethylpropan-2-amine; 1-[(2R)-1-{4-[(3)-cholest-5-en-3-yloxy]butoxy}-3-(octyloxy)propan-2-yl]guanidine; 1-[(2R)-1-{7-[(3β)-cholest-5-en-3-yloxy]heptyloxy}-N,N-dimethyl-3-[(9Z,12Z)-octadeca-9,12-dien-1-yloxy]propan-2-amine; 1-[(2R)-1-{4-[(3β)-cholest-5-en-3-yloxy]butoxy}-N,N-dimethyl-3-[(9Z,12Z)-octadeca-9,12-dien-1-yloxy]propan-2-amine; (2S)-1-({6-[(3β)-cholest-5-en-3-yloxy]hexyl}oxy)-N,N-dimethyl-3-[(9Z)-octadec-9-en-1-yloxy]propan-2-amine; (3β)-3-[6-{[(2S)-3-[(9Z)-octadec-9-en-1-yloxyl]-2-(pyrrolidin-1-yl)propyl]oxy}hexyl)oxy]cholest-5-ene; (2R)-1-{4-[(3β)-cholest-5-en-3-yloxy]butoxy}-3-(octyloxy)propan-2-amine; (2R)-1-({8-[(3β)-cholest-5-en-3-yloxy]octyl}oxy)-N,N-dimethyl-3-(pentyloxy)propan-2-amine; (2R)-1-({8-[(3β)-cholest-5-en-3-yloxy]octyl}oxy)-3-(heptyloxy)-N,N-dimethylpropan-2-amine; (2R)-1-({8-[(3β)-cholest-5-en-3-yloxy]octyl}oxy)-N,N-dimethyl-3-[(2Z)-pent-2-en-1-yloxy]propan-2-amine; (2S)-1-butoxy-3-({8-[(3β)-cholest-5-en-3-yloxy]octyl}oxy)-N,N-dimethylpropan-2-amine; (2S-1-({8-[(3β)-cholest-5-en-3-yloxy]octyl}oxy)-3-[2,2,3,3,4,4,5,5,6,6,7,7,8,8,9,9-hexadecafluorononyl)oxy]-N,N-dimethylpropan-2-amine; 2-amino-2-{[(9Z,12Z)-octadeca-9,12-dien-1-yloxy]methyl}propane-1,3-diol; 2-amino-3-({9-[(3β,8ξ,9ξ,14ξ,17ξ,20ξ)-cholest-5-en-3-yloxy]nonyl}oxy)-2-{[(9Z,12Z)-octadeca-9,12-dien-1-yloxy]methyl}propan-1-ol; 2-amino-3-({6-[(3β,8ξ,9ξ,14ξ,17ξ,20ξ)-cholest-5-en-3-yloxy]hexyl}oxy)-2-{[(9Z)-octadec-9-en-1-yloxy]methyl}propan-1-ol; (20Z,23Z)-N,N-dimethylnonacosa-20,23-dien-10-amine; (17Z,20Z)-N,N-dimethylhexacosa-17,20-dien-9-amine; (16Z,19Z)-N,N-dimethylpentacosa-16,19-dien-8-amine; (13Z,16Z)-N,N-dimethyldocosa-13,16-dien-5-amine; (12Z,15Z)-N,N-dimethylhenicosa-12,15-dien-4-amine; (14Z,17Z)-N,N-dimethyltricosa-14,17-dien-6-amine; (15Z,18Z)-N,N-dimethyltetracosa-15,18-dien-7-amine; (18Z,21Z)-N,N-dimethylheptacosa-18,21-dien-10-amine; (15Z,18Z)-N,N-dimethyltetracosa-15,18-dien-5-amine; (14Z,17Z)-N,N-dimethyltricosa-14,17-dien-4-amine; (19Z,22Z)-N,N-dimethyloctacosa-19,22-dien-9-amine; (18Z,21Z)-N,N-dimethylheptacosa-18,21-dien-8-amine; (17Z,20Z)-N,N-dimethylhexacosa-17,20-dien-7-amine; (16Z,19Z)-N,N-dimethylpentacosa-16,19-dien-6-amine; (22Z,25Z)-N,N-dimethylhentriaconta-22,25-dien-10-amine; (21Z,24Z)-N,N-dimethyltriaconta-21,24-dien-9-amine; (18Z)-N,N-dimethylheptacos-18-en-10-amine; (17Z)-N,N-dimethylhexacos-17-en-9-amine; (19Z,22Z)-N,N-dimethyloctacosa-19,22-dien-7-amine; N,N-dimethylheptacosan-10-amine; (20Z,23Z)-N-ethyl-N-methylnonacosa-20,23-dien-10-amine; 1-[(11Z,14Z)-1-nonylicosa-11,14-dien-1-yl]pyrrolidine; (20Z)-N,N-dimethylheptacos-20-en-10-amine; (15Z)-N,N-dimethylheptacos-15-en-10-amine; (14Z)-N,N-dimethylnonacos-14-en-10-amine; (17Z)-N,N-dimethylnonacos-17-en-10-amine; (24Z)-N,N-dimethyltritriacont-24-en-10-amine; (20Z)-N,N-dimethylnonacos-20-en-10-amine; (22Z)-N,N-dimethylhentriacont-22-en-10-amine; (16Z)-N,N-dimethylpentacos-16-en-8-amine; (12Z,15Z)-N,N-dimethyl-2-nonylhenicosa-12,15-dien-1-amine; (13Z,16Z)-N,N-dimethyl-3-nonyldocosa-13,16-dien-1-amine; N,N-dimethyl-1-[(1S,2R)-2-octylcyclopropyl]heptadecan-8-amine; 1-[(1S,2R)-2-hexylcyclopropyl]-N,N-dimethylnonadecan-10-amine; N,N-dimethyl-1-[(1S,2R)-2-octylcyclopropyl]nonadecan-10-amine; N,N-dimethyl-21-[(1S,2R)-2-octylcyclopropyl]henicosan-10-amine; N,N-dimethyl-1-[(1S,2S)-2-{[(1R,2R)-2-pentylcyclopropyl]methyl}cyclopropyl]nonadecan-10-amine; N,N-dimethyl-1-[(1S,2R)-2-octylcyclopropyl]hexadecan-8-amine; N,N-dimethyl-1-[(1R,2S)-2-undecylcyclopropyl]tetradecan-5-amine; N,N-dimethyl-3-{7-[(1S,2R)-2-octylcyclopropyl]heptyl}dodecan-1-amine; 1-[(1R,2S)-2-heptylcyclopropyl]-N,N-dimethyloctadecan-9-amine; 1-[(1S,2R)-2-decylcyclopropyl]-N,N-dimethylpentadecan-6-amine; N,N-dimethyl-1-[(1S,2R)-2-octylcyclopropyl]pentadecan-8-amine; (11E,20Z,23Z)-N,N-dimethylnonacosa-11,20,23-trien-10-amine; 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dioleoyl-4-dimethylaminomethyl-[1,3]-dioxolane (DO-K-DMA), 2,2-distearoyl-4-dimethylaminomethyl-[1,3]-dioxolane (DS-K-DMA), 2,2-dilinoleyl-4-N-morpholino-[1,3]-dioxolane (DLin-K-MA), 2,2-Dilinoleyl-4-trimethylamino-[1,3]-dioxolane chloride (DLin-K-TMA·Cl), 2,2-dilinoleyl-4,5-bis(dimethylaminomethyl)-[1,3]-dioxolane (DLin-K2-DMA), 2,2-dilinoleyl-4-methylpiperzine-[1,3]-dioxolane (D-Lin-K-N-methylpiperzine), DLen-C2K-DMA, 7-DLen-C2K-DMA, DPan-C2K-DMA, DPan-C3K-DMA, DLen-C2K-DMA, 7-DLen-C2K-DMA, DPan-C2K-DMA, TLinDMA, C2-TLinDMA, C3-TLinDMA, 1,2-di-7-linolenyloxy-N,N-dimethylaminopropane (7-DLenDMA), 1,2-dilinoleyloxy-(N,N-dimethyl)-butyl-4-amine (C2-DLinDMA), 1,2-dilinoleoyloxy-(N,N-dimethyl)-butyl-4-amine (C2-DLinDAP), CP-LenMC3, CP-7-LenMC3, CP-MC3, CP-DLen-C2K-DMA, CP-7DLen-C2K-DMA, CP-C2K-DMA, CP-DODMA, CP-DPetroDMA, CP-DLinDMA, CP-DLenDMA, CP-7DLenDMA, 1,2-dioeylcarbamoyloxy-3-dimethylaminopropane (DO-C-DAP), 1,2-dimyristoleoyl-3-dimethylaminopropane (DMDAP), 1,2-dioleoyl-3-trimethylaminopropane chloride (DOTAP·Cl), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt (DLin-TAP·Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), and 1,2-N,N′-dilinoleylcarbamyl-3-dimethylaminopropane (DLincarbDAP); as well as pharmaceutically acceptable salts thereof, and stereoisomers of any of the foregoing.

As used herein, the term “non-cationic lipid” refers to any uncharged, anionic, or zwitterionic lipid. At physiological pH, such lipids include, for example, diacylphosphatidylcholine, diacylphosphatidylethanolamine, ceramide, sphingomyelin, cephalin, cholesterol, cerebrosides, diacylglycerols, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids. In some embodiments, the non-cationic lipid used in the instant disclosure is 1,2-dioleoyl-sn-glycero-3-phosphocholine (DOPC), 1,2-Distearoyl-sn-glycero-3-phosphocholine (DSPC), and/or 1,2-Dioleoyl-sn-glycero-3-phosphoethanolamine (DOPE). In embodiments, the non-cationic lipid is cholesterol (CHE) and/or β-sitosterol.

Exemplary zwitterionic non-cationic lipids include the following phospholipids. 16:0-18:0 PC, also known as 1-palmitoyl-2-stearoyl-sn-glycero-3-phosphocholine, has the following structure:

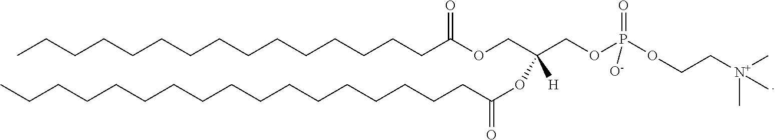 [SMILES: CCCCCCCCCCCCCCCCCC(=O)O[C@H](COC(=O)CCCCCCCCCCCCCCC)COP(=O)([O-])OCC[N+](C)(C)C]

16:0/16:1(9Z)-PC, also known as 1-(1-enyl-palmitoyl)-2-palmitoleoyl-sn-glycero-3-phosphocholine, has the following structure:

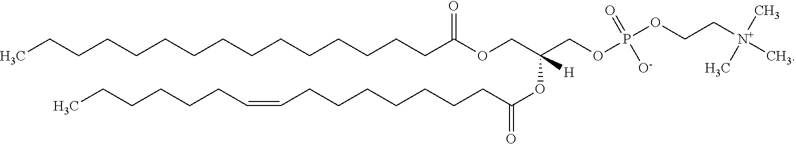 [SMILES: CCCCCC/C=C\CCCCCCCC(=O)O[C@H](COC(=O)CCCCCCCCCCCCCCC)COP(=O)([O-])OCC[N+](C)(C)C]

16:0-18:2 PC, also known as 1-palmitoyl-2-linoleoyl-sn-glycero-3-phosphocholine, has the following structure:

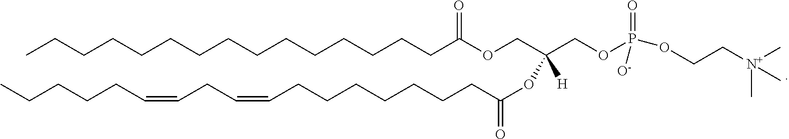 [SMILES: CCCCC/C=C\C/C=C\CCCCCCCC(=O)O[C@H](COC(=O)CCCCCCCCCCCCCCC)COP(=O)([O-])OCC[N+](C)(C)C]

18:0-18:1(9Z)-PC, also known as 1-stearoyl-2-oleoyl-sn-glycero-3-phosphocholine, has the following structure:

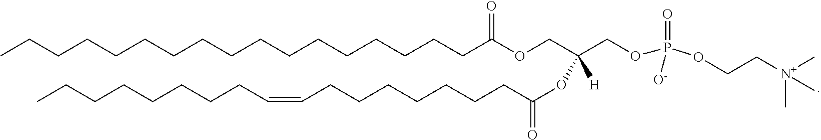 [SMILES: CCCCCCCC/C=C\CCCCCCCC(=O)O[C@H](COC(=O)CCCCCCCCCCCCCCCCC)COP(=O)([O-])OCC[N+](C)(C)C]

18:0-18:2(9Z,12Z)-PC, also known as 1-Octadecanyl-2-(9Z,12Z-octadecadienoyl)-sn-glycero-3-phosphocholine, has the following structure:

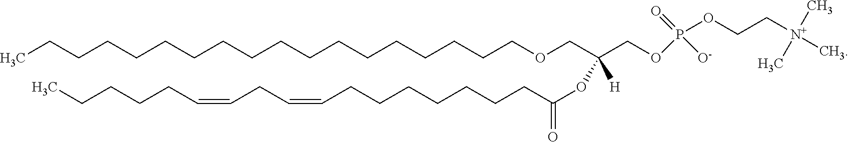 [SMILES: CCCCC/C=C\C/C=C\CCCCCCCC(=O)O[C@H](COCCCCCCCCCCCCCCCCCC)COP(=O)([O-])OCC[N+](C)(C)C]

18:1-18:2(9Z,12Z)-PC, also known as 1-(9Z,12Z-octadecadienoyl)-2-(9Z-octadecenoyl)-glycero-3-phosphocholine, has the following structure:

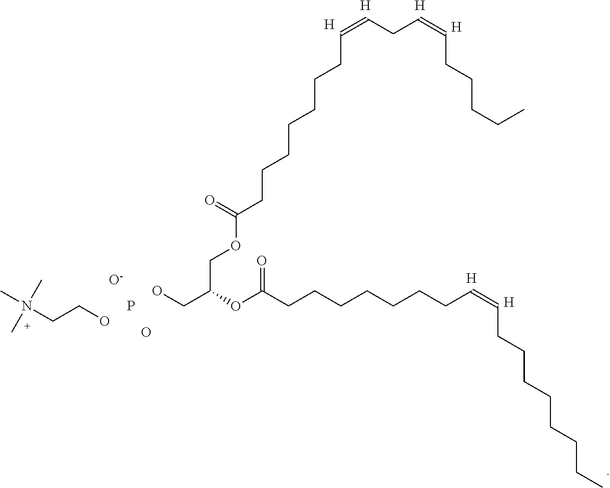

In some embodiments, the non-cationic lipid present in the lipid particles comprises or consists of a mixture of one or more phospholipids and cholesterol or a derivative thereof.

The term “lipid nanoparticle (LNP)” as used herein refers to different types of compositions of nano-scale particles, wherein the particles comprising lipids function as carriers across cell membranes and biological barriers and deliver compounds to targeted cells and tissues of humans and other organisms. As used herein, “lipid nanoparticles” of the instant disclosure may further comprise additional lipids and other components. Other lipids may be included for a variety of purposes, such as to prevent lipid oxidation or to attach ligands onto the lipid nanoparticle surface. Any of a number of lipids may be present in lipid nanoparticles of the present disclosure, including amphipathic, neutral, cationic, and anionic lipids. Such lipids can be used alone or in combination, and can also include bilayer stabilizing components such as polyamide oligomers (see, e.g., U.S. Pat. No. 6,320,017), peptides, proteins, detergents, lipid-derivatives, such as PEG coupled to phosphatidylethanolamine and PEG conjugated to ceramides (see, e.g., U.S. Pat. No. 5,885,613).

As used herein, a “PEG” conjugated lipid that inhibits aggregation of particles refers to one or more of a polyethyleneglycol (PEG)-lipid conjugate, a polyamide (ATTA)-lipid conjugate, and a mixture thereof. In one aspect, the PEG-lipid conjugate is one or more of a PEG-dialkyloxypropyl (DAA), a PEG-diacylglycerol (DAG), a PEG-phospholipid, a PEG-ceramide, and a mixture thereof. In one aspect, the PEG-DAG conjugate is one or more of a PEG-dilauroylglycerol (C₁₂), a PEG-dimyristoylglycerol (C₁₄), a PEG-dipalmitoylglycerol (C₁₆), and a PEG-distearoylglycerol (C₁₈). In one aspect, the PEG-DAA conjugate is one or more of a PEG-dilauryloxypropyl (C₁₂), a PEG-dimyristyloxypropyl (C₁₄), a PEG-dipalmityloxypropyl (C₁₆), and a PEG-distearyloxypropyl (C₁₈). In some embodiments, PEG is 2-dimyristoyl-rac-glycero-3-methoxypolyethylene glycol-2000 (PEG-DMG) and/or 1,2-distearoyl-rac-glycero-3-methoxypolyethylene glycol-2000 (PEG-DSG).

The term “N/P ratio” as used herein refers to the (N)itrogen-to-(P)hosphate molar ratio between the cationic amino lipid and negatively charged phosphate groups of the nucleic acid.

The “polydispersity index” or “PDI” as used herein is a measure of the heterogeneity of a sample based on size. Polydispersity can occur due to size distribution in a sample or agglomeration or aggregation of the sample during isolation or analysis.

The “zeta potential” or “surface charge” as used herein refers to the degree of electrostatic repulsion between adjacent, similarly charged particles in a dispersion. For molecules and particles that are small enough, a high zeta potential will confer stability, i.e., the solution or dispersion will resist aggregation.

As used herein, the term nucleic acid “cargo” refers to the intended nucleic acid for delivery to the cell or tissue (in embodiments, a therapeutic nucleic acid for delivery to the cell or tissue).

As used herein, the term “nucleic acid-lipid nanoparticle” refers to lipid nanoparticles as described above that associate with or encapsulate one or more nucleic acids to deliver one or more nucleic acid cargoes to a tissue.

As used herein, “encapsulated” can refer to a nucleic acid-lipid nanoparticle formulation that provides a nucleic acid with full encapsulation, partial encapsulation, association by ionic or van der Waals forces, or all of the aforementioned. In one embodiment, the nucleic acid is fully encapsulated in the nucleic acid-lipid nanoparticle.

As used herein, “nucleic acid” refers to a synthetic or naturally occurring RNA or DNA, or derivatives thereof. In one embodiment, a cargo and/or agent of the instant disclosure is a nucleic acid, such as a double-stranded RNA (dsRNA). In one embodiment, the nucleic acid or nucleic acid cargo is a single-stranded DNA or RNA, or double-stranded DNA or RNA, or DNA-RNA hybrid. For example, a double-stranded DNA can be a structural gene, a gene including control and termination regions, or a self-replicating system such as a viral or plasmid DNA. A double-stranded RNA can be, e.g., a dsRNA or another RNA interference reagent. A single-stranded nucleic acid can be, e.g., an mRNA, an antisense oligonucleotide, ribozyme, a microRNA, or triplex-forming oligonucleotide. In certain embodiments, the nucleic acid or nucleic acid cargo may comprise a modified RNA, wherein the modified RNA is one or more of a modified mRNA, a modified antisense oligonucleotide and a modified siRNA. In some embodiments, a nucleic acid cargo of the instant disclosure includes or is a modified mRNA that encodes a nucleic acid modulating controller.

As used herein, the term “modified nucleic acid” refers to any non-natural nucleic acid, including but not limited to those selected from the group comprising 2′-O-methyl modified nucleotides, a nucleotide comprising a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide; internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

As used herein, the term “nucleic acid modulating controller” refers to a mRNA that encodes for protein controller components, though reference to “nucleic acid modulating controller” can also refer to the mRNA-expressed protein controller components themselves. In certain embodiments, the mRNA-encoded protein controller components include Zinc-Finger proteins (ZFPs) or other forms of DNA or RNA binding domains (DBDs or RBDs) that are associated with (and optionally tethered to) one or more epigenetic regulators or nucleases (the epigenetic regulators or nucleases are generally referred to as effectors, effector domains, or effector moieties). Without wishing to be bound by theory, an advantage of a nucleic acid modulating controller as described herein is that it provides durable gene programming only at the confluence of (1) where the nucleic acid modulating controller-encoding mRNA is expressed, (2) where nucleic acid binding of the ZFP or other nucleic acid binding domain occurs and (3) where the associated effector domain is able to exert activity (i.e. where the effector domain is capable of changing the epigenomic state (e.g., in the instance of an epigenomic controller)).

As used herein, the term “effector moiety” or “effector domain” refers to a domain that is capable of altering the expression of a target gene when localized to an appropriate site in a cell, e.g., in the nucleus of a cell. In some embodiments, an effector moiety recruits components of the transcription machinery. In some embodiments, an effector moiety inhibits recruitment of components of transcription factors or expression repressing factors. In some embodiments, an effector moiety comprises an epigenetic modifying moiety (e.g., epigenetically modifies a target DNA sequence). Specific examples of effector moieties include, without limitation, effectors capable of binding Krueppel-associated box (KRAB) domains (KRAB is a domain of around 75 amino acids that is found in the N-terminal part of about one third of eukaryotic Krueppel-type C2H2 zinc finger proteins (ZFPs)) and the engineered prokaryotic DNA methyltransferase MQ1, among others.

As used herein, “epigenetic modifying moiety” refers to a domain that alters: i) the structure, e.g., two-dimensional structure, of chromatin; and/or ii) an epigenetic marker (e.g., one or more of DNA methylation, histone methylation, histone acetylation, histone sumoylation, histone phosphorylation, and RNA-associated silencing), when the epigenetic modifying moiety is appropriately localized to a nucleic acid (e.g., by a targeting moiety). In some embodiments, an epigenetic modifying moiety comprises an enzyme, or a functional fragment or variant thereof, that affects (e.g., increases or decreases the level of) one or more epigenetic markers. In some embodiments, an epigenetic modifying moiety comprises a DNA methyltransferase, a histone methyltransferase, CREB-binding protein (CBP), or a functional fragment of any thereof.

As used herein, the term “expression control sequence” refers to a nucleic acid sequence that increases or decreases transcription of a gene and includes (but is not limited to) a promoter and an enhancer. An “enhancing sequence” refers to a subtype of expression control sequence and increases the likelihood of gene transcription. A “silencing or repressor sequence” refers to a subtype of expression control sequence and decreases the likelihood of gene transcription.

As used herein, the term “expression repressor” refers to an agent or entity with one or more functionalities that decreases expression of a target gene in a cell and that specifically binds to a DNA sequence (e.g., a DNA sequence associated with a target gene or a transcription control element operably linked to a target gene). In certain embodiments, an expression repressor comprises at least one targeting moiety and optionally one effector moiety.

As used herein, the term “targeting moiety” means an agent or entity that specifically targets, e.g., binds, a genomic sequence element (e.g., an expression control sequence or anchor sequence; promoter, enhancer or CTCF site). In some embodiments, the genomic sequence element is proximal to and/or operably linked to a target gene (e.g., MYC).

As used herein, “localization” refers to the position of a lipid, peptide, or other component of a lipid particle of the instant disclosure, within an organism and/or tissue. In some embodiments, localization can be detectible in individual cells. In some embodiments a label can be used for detecting localization, e.g., a fluorescent label, optionally a fluorescently labeled lipid, optionally Cy7. In some embodiments, the label of the lipid nanoparticle may be a quantum dot, or the lipid detectible by stimulated Raman scattering. In other embodiments, the label is any fluorophore known in the art, i.e. with excitation and emission in the ultraviolet, visible, or infrared spectra. In some embodiments the localization is detected or further corroborated by immunohistochemistry or immunofluorescence.

As used herein, the term “activity” refers to any detectable effect that is mediated by a component or composition of the instant disclosure. In embodiments, “activity” as used herein, can refer to a measurable (whether directly or by proxy) effect, e.g., of a cargo of the instant lipid particles of the disclosure. Examples of activity include, without limitation, the intracellular expression and resulting effect(s) of a nucleic acid cargo (e.g., a mRNA, a CRISPR/Cas system, a RNAi agent, a nucleic acid modulating controller, etc.), which can optionally be measured at a cellular, tissue, organ and/or organismal level.

As used herein, “multidosing” refers to two or more doses of a lipid nanoparticle formulation given as part of a therapeutic regimen to a subject.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, “administration” to a subject may include parenteral administration, optionally for intravenous injection, inhalation, intravenous, intra-arterial, intratracheal, topical, or involve direct injection into a tissue.

The term “treating” includes the administration of compositions to prevent or delay the onset of the symptoms, complications, or biochemical indicia of a disease (e.g., cancer, including, e.g., tumor formation, growth and/or metastasis), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

As used herein, a “pharmaceutical composition” comprises a pharmacologically effective amount of a lipid particle, optionally a nucleic-acid lipid nanoparticle (NLNP) and a pharmaceutically acceptable carrier. As used herein, “pharmacologically effective amount,” “therapeutically effective amount” or simply “effective amount” refers to that amount of nucleic acid effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to induce at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the disclosure.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control.

In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A to 1J show an exemplary formula and exemplary structures of reversible zwitterionic lipids as disclosed herein. FIG. 1A shows an exemplary formula for reversible zwitterionic lipids of the instant disclosure. FIG. 1B shows an exemplary structure of 3-(dioctylamino)propyl nonyl hydrogen phosphate (OMGT-014). FIG. 1C shows an exemplary structure of 4-(dioctylamino)but-2-yn-1-yl nonyl hydrogen phosphate (OMGT-047). FIG. 1D shows an exemplary structure of 4-(dioctylamino)butyl (3-propylhexyl) hydrogen phosphate (OMGT-043). FIG. 1E shows an exemplary structure of 4-(dioctylamino)butyl (7-methyloctyl) hydrogen phosphate (OMGT-042). FIG. 1F shows an exemplary structure of 2-butylhexyl (4-(dioctylamino)butyl) hydrogen phosphate (OMGT-044). FIG. 1G shows an exemplary structure of 6-(dioctylamino)hexyl nonyl hydrogen phosphate (OMGT-055). FIG. 1H shows an exemplary structure of (E)-4-(dioctylamino)but-2-en-1-yl nonyl hydrogen phosphate (OMGT-045). FIG. 1I shows an exemplary structure of (Z)-4-(dioctylamino)butyl non-3-en-1-yl hydrogen phosphate (OMGT-040). FIG. 1J shows an exemplary structure of (Z)-4-(dioctylamino)but-2-en-1-yl nonyl hydrogen phosphate (OMGT-046).

### DETAILED DESCRIPTION

The present disclosure is based, at least in part, upon the discovery of novel reversible zwitterionic lipids having an ionizable tertiary amine that is connected to an electron withdrawing phosphate group via a linker including ≥C₃, which together form a zwitterion that is reversable at different pH values. Additionally, the reversible zwitterionic lipids disclosed herein demonstrate an increased pKa of the ionizable tertiary amine relative to prior art phospholipids (e.g., iPhos) that only have a C₂ linker. Without being bound by theory, it is believed that there is an electrostatic interaction between the ionizable tertiary amine and the phosphate group, and that increasing the length of the linker between the tertiary amine and the phosphate group to ≥C₃ increases the pKa of the tertiary amine by increasing the distance between the ionizable tertiary amine and the electron withdrawing phosphate group, while simultaneously decreasing the electrostatic interaction between the amine and phosphate groups.

Furthermore, the reversible zwitterionic lipids disclosed herein may include an ionizable tertiary amine (e.g., pH-titratable) head group, a linker, and a phosphate group, where the ionizable tertiary amine head group includes two hydrocarbon chains (e.g., C₇-C₂₂ alkyl, alkenyl, or alkynyl) and the phosphate group includes one hydrocarbon chain (e.g., C₃-C₂₂ alkyl, alkenyl, or alkynyl), where each of the aforementioned hydrocarbon chains independently has 0 to 3 (e.g., 0, 1, 2, or 3) double bonds, and ether, ester, or ketal linkages between the ionizable amine head group and hydrocarbon chains.

The reversible zwitterionic lipids disclosed herein have advantageous properties when used in lipid particles for the in vivo delivery of a therapeutic agent(s) because the ionizable tertiary amine may become protonated when the reversible zwitterionic lipid enters the endosome to form a zwitterionic head and the three hydrocarbon chains are able to form a cone shaped structure when inserted into the endosomal membrane that facilitates hexagonal transformation. Additionally, the present disclosure provides reversible zwitterionic lipids having an ionizable tertiary amine with an increased pKa relative to prior art lipids that only include a C₂ linker between a tertiary amine and a phosphate group (see e.g., the iPhos chemical structures disclosed in Liu et al. 2021 Nat. Mater. 20(5): 701-710) that can advantageously be incorporated into lipid nanoparticles to improve endosomal escape and thereby increase the efficiency of delivery of a therapeutic agent(s) such as, for example, nucleic acids (e.g., siRNA, ASO, tRNA, miRNA, mRNA, DNA, and the like), proteins, peptides, and other macromolecules, which do not have the ability to easily cross a cell membrane.

In certain aspects, the disclosure provides methods of synthesizing the novel reversible zwitterionic lipids. In certain aspects, the disclosure provides mixed lipid particle compositions and formulations including the novel ionizable lipid(s) disclosed herein, as well as associated methods for delivery of lipid particle-associated molecular cargoes to the cells of a subject. In certain aspects, nucleic-acid lipid nanoparticles are provided that preferentially localize to and deliver associated nucleic acid cargoes to the liver, lung, skin, tumor or other tissue of a subject, with delivery optionally occurring to various types of tissue and/or population(s) of cells within a tissue of a subject.

LNPs used for the delivery of nucleic acids to cells have typically been composed of four main components. An ionizable or cationic lipid for mRNA encapsulation, amphipathic helper phospholipids for increased efficacy, cholesterol for structural stability and polyethylene glycol (PEG)-lipids for steric stability. Such LNPs can be considered as “one ionizable lipid-only LNPs”, or “single LNPs”. Conventionally, effective intracellular delivery materials have relied on an optimal balance of ionizable amines to bind and release RNAs (pKa between 6.0 and 6.5) and nanoparticle-stabilizing hydrophobicity. Thus, there has been an exhaustive focus on developing ionizable lipids, which have been proven to be highly effective delivery platforms for liver and hepatocytes. However, changing the chemical structure of the ionizable/cationic lipid to achieve different pKa values and generating libraries, although validated, is a time consuming, investment heavy and labor-intensive exercise.

The present disclosure provides reversible zwitterionic lipids having an ionizable tertiary amine that is connected to an electron withdrawing phosphate group via a ≥C₃ linker configured to increase the pKa of the tertiary amine by increasing the distance between the ionizable tertiary amine and the electron withdrawing phosphate group. Advantageously, it was discovered that increasing the pKa of the tertiary amine beneficially impacts its ionization at specific pH with a subsequent increase in the ability of the reversible zwitterionic lipid to enhance endosomal escape efficiency of a lipid particle(s) into which the reversible zwitterionic lipid is incorporated. For example, lipid particles or lipid nanoparticles that include reversible zwitterionic lipids as disclosed herein display improved endosomal escape and thereby increased efficiency of delivery of therapeutic agents. The novel reversible zwitterionic lipids disclosed herein have the general structure set forth in Formula I below and include the (R) and/or (S) enantiomers thereof.

In embodiments, the techniques herein provide improved lipid-based compositions for the delivery of therapeutic agents, in particular, nucleic acid therapeutic agents. As disclosed herein, these lipid-based compositions are effective in increasing the efficiency of cargo release from lipid-based composition such as LNPs. Furthermore, the present disclosure demonstrates that the activity of these improved lipid-based compositions is dependent on the presence of certain novel reversible zwitterionic lipids disclosed herein.

It is contemplated within the scope of the disclosure that the lipid-based compositions including the reversible zwitterionic lipids disclosed herein may be used for a variety of purposes such as, for example, the delivery of encapsulated therapeutic agents to cells, in vitro and/or in vivo. In this regard, the present disclosure provides methods of treating diseases or disorders in a subject in need thereof by contacting the subject with the lipid-based compositions disclosed herein when combined with the suitable therapeutic agent such as, for example, nucleic acids (e.g., siRNA, ASO, tRNA, miRNA, mRNA, DNA, and the like), proteins, peptides, and other macromolecules.

In embodiments, the lipid-based compositions disclosed herein are particularly useful for the delivery of nucleic acid therapeutics (e.g., siRNA, ASO, tRNA, miRNA, mRNA, DNA, and the like). The lipid-based compositions disclosed herein may be used to modulate the expression of target genes and proteins both in vitro and in vivo by contacting tissues/cells with a lipid-based composition including a lipid as disclosed herein carrying a cargo such as a therapeutic nucleic acid (e.g., an siRNA) that may reduce expression of a desired target gene.

The techniques herein provide reversible zwitterionic lipids that enable the formulation of pharmaceutical compositions for the in vitro or in vivo delivery of therapeutic agents such as, for example, nucleic acids (e.g., siRNA, ASO, tRNA, miRNA, mRNA, DNA, and the like), proteins, peptides, and other macromolecules.

Exemplary embodiments of the reversible zwitterionic lipids of the present disclosure, as well as lipid-based compositions comprising the same, as well as their synthesis and use to deliver therapeutic agents is described in further detail below.

### Lipids

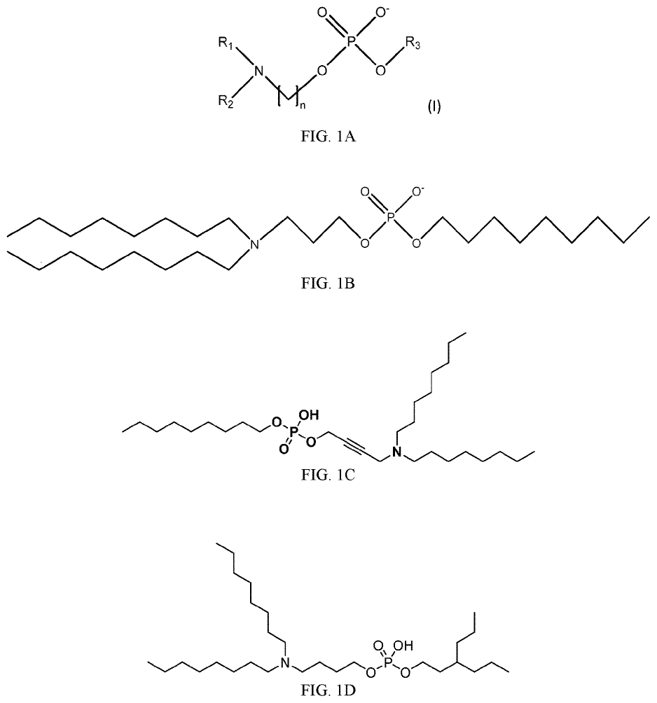

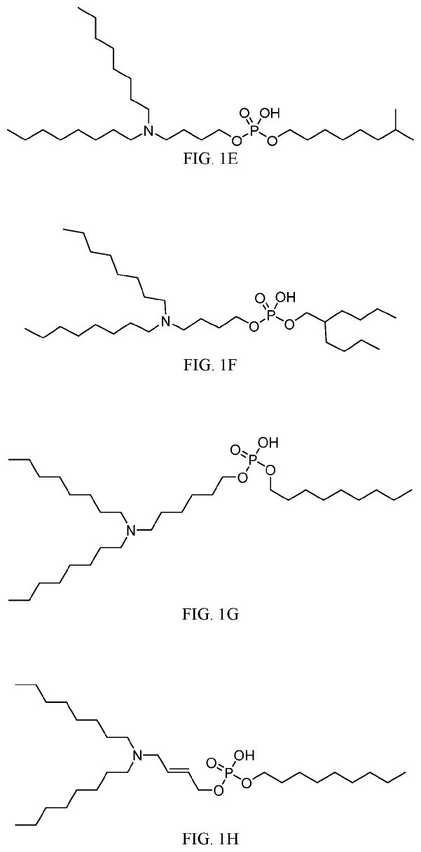

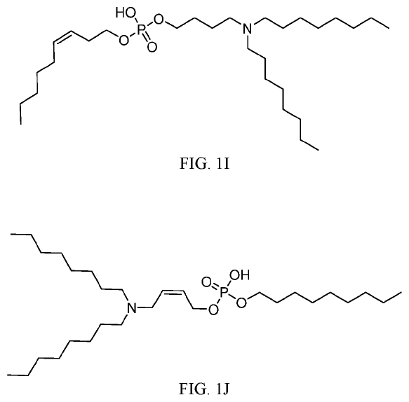

The present disclosure provides novel reversible zwitterionic lipids of the general structure of Formula I shown in FIG. 1A. The reversible zwitterionic lipids have design features including a backbone comprising an ionizable tertiary amine (e.g., head group), an electron withdrawing phosphate group, and an at least C₃ linker, wherein the linker connects the ionizable tertiary amine to the phosphate group. Without being bound by theory, the ≥C₃ linker is configured to increase the pKa of the tertiary amine by increasing the distance between the ionizable tertiary amine and the electron withdrawing phosphate group. Additional design features include two C₇-C₂₂ hydrocarbon tails (e.g., alkyl, alkenyl, or alkynyl, optionally either or both tails include an optionally substituted heterocycle (e.g., a heterocyclic ring), or both tails may join to form an optionally substituted heterocycle (e.g., a heterocyclic ring), and the like) connected to the tertiary amine, and a C₃-C₂₂ hydrocarbon (e.g., alkyl, alkenyl, or alkynyl) group connected to the phosphate group. An exemplary reversible zwitterionic lipid as disclosed herein is shown in FIG. 1B.

Certain aspects of the present disclosure provide novel reversible zwitterionic lipids that may be advantageously used in lipid-based compositions of the present disclosure for the in vivo delivery of therapeutic agents to tissues/cells.

It is contemplated within the scope of the disclosure that the reversible zwitterionic lipid comprises a racemic mixture or a mixture of one or more diastereomers. In some embodiments, the cationic lipid is enriched in one enantiomer, such that the cationic lipid comprises at least about 55%, 60%, 65%, 70%, 75%, 80%, 85%, 90%, or 95% enantiomeric excess. In some embodiments, the cationic lipid is enriched in one diastereomer, such that the cationic lipid comprises at least about 55%, 60%, 65%, 70%, 75%, 80%, 85%, 90%, or 95% diastereomeric excess. In some embodiments, the cationic lipid is chirally pure (e.g., comprises a single optical isomer). In some embodiments, the cationic lipid is enriched in one optical isomer (e.g., an optically active isomer), such that the cationic lipid comprises at least about 55%, 60%, 65%, 70%, 75%, 80%, 85%, 90%, or 95% isomeric excess. The disclosure provides the synthesis of reversible zwitterionic lipids of Formula I as a racemic mixture or in optically pure form.

As used herein, the term “salts” includes any anionic and cationic complex, such as the complex formed between a cationic lipid disclosed herein and one or more anions. Examples of anions include, but are not limited to, inorganic and organic anions such as, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof. In particular embodiments, the salts of the cationic lipids disclosed herein are crystalline salts.

As used herein, the term “alkyl” includes a straight chain or branched, noncyclic or cyclic, saturated aliphatic hydrocarbon containing from 1 to 24 carbon atoms. Representative saturated straight chain alkyls include, but are not limited to, methyl, ethyl, n-propyl, n-butyl, n-pentyl, n-hexyl, and the like, while saturated branched alkyls include, without limitation, isopropyl, sec-butyl, isobutyl, tert-butyl, isopentyl, and the like. Representative saturated cyclic alkyls include, but are not limited to, cyclopropyl, cyclobutyl, cyclopentyl, cyclohexyl, and the like, while unsaturated cyclic alkyls include, without limitation, cyclopentenyl, cyclohexenyl, and the like.

As used herein, the term “alkenyl” includes an alkyl, as defined above, containing at least one double bond between adjacent carbon atoms. Alkenyls include both cis and trans isomers. Representative straight chain and branched alkenyls include, but are not limited to, ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. Cyclic alkenyls are also contemplated for the lipids of the instant disclosure.

As used herein, the term “alkynyl” includes any alkyl or alkenyl, as defined above, which additionally contains at least one triple bond between adjacent carbons. Representative straight chain and branched alkynyls include, without limitation, acetylenyl, propynyl, 1-butynyl, 2-butynyl, 1-pentynyl, 2-pentynyl, 3-methyl-1 butynyl, and the like.

As used herein, the term “acyl” includes any alkyl, alkenyl, or alkynyl wherein the carbon at the point of attachment is substituted with an oxo group, as defined below. The following are non-limiting examples of acyl groups: C(═O)alkyl, C(═O)alkenyl, and C(═O)alkynyl.

As used herein, the term “heterocycle” includes a monocyclic (e.g., 5-, 6-, 7-membered, and the like), bicyclic (e.g., 7-, 8-, 9-, 10-membered, and the like), or heterocyclic ring which is either saturated, unsaturated, or aromatic, and which contains from 1 or 2 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include, but are not limited to, heteroaryls as defined below, as well as morpholinyl, pyrrolidinonyl, pyrrolidinyl, piperidinyl, piperizynyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

As used herein, the terms “optionally substituted alkyl”, “optionally substituted alkenyl”, “optionally substituted alkynyl”, “optionally substituted acyl”, and “optionally substituted heterocycle” mean that, when substituted, at least one hydrogen atom is replaced with a substituent. In the case of an oxo substituent (═O), two hydrogen atoms are replaced. In this regard, substituents include, but are not limited to, oxo, halogen, heterocycle, —CN, —NRxRy, —NRxC(═O)Ry, —NRxSO2Ry, —C(═O)Rx, —C(═O)ORx, —C(═O)NRxRy, —SOnRx, and —SOnNRxRy, wherein n is 0, 1, or 2, Rx and Ry are the same or different and are independently hydrogen, alkyl, or heterocycle, and each of the alkyl and heterocycle substituents may be further substituted with one or more of oxo, halogen, —OH, —CN, alkyl, —ORx, heterocycle, —NRxRy, —NRxC(═O)Ry, —NRxSO2Ry, —C(═O)ORx, —C(═O)ORx, —C(═O)NRxRy, —C(O—R1)(O—R2), —SOnRx, and —SOnNRxRy. The term “optionally substituted,” when used before a list of substituents, means that each of the substituents in the list may be optionally substituted as described herein.

As used herein, the term “halogen” includes fluoro, chloro, bromo, and iodo.

In embodiments, the present disclosure provides a reversible zwitterionic lipid of general Formula I having the following structure:

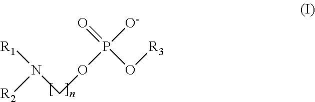

or salts thereof, wherein:
R₁ and R₂ are either the same or different and are independently and optionally substituted C₇-C₂₂ alkyl, C₇-C₂₂ alkenyl, or C₇-C₂₂ alkynyl, optionally R₁, R₂, or R₁ and R₂ are an optionally substituted heterocycle or R₁ and R₂ may join to form an optionally substituted heterocycle;
R₃ is optionally substituted C₃-C₂₂ alkyl, C₃-C₂₂ alkenyl, or C₃-C₂₂ alkynyl; and
n is 3, 4, 5, 6, 7, 8, 9, 10, 11, 12, 13, 14, 15, 16, 17, 18, 19, 20, 21, or 22.

In some embodiments, R₁ and R₂ are each independently C₇-C₈ alkyl, C₇-C₉ alkyl, C₇-C₁₀ alkyl, C₇-C₁₁ alkyl, C₇-C₁₂ alkyl, C₇-C₁₃ alkyl, C₇-C₁₄ alkyl, C₇-C₁₅ alkyl, C₇-C₁₆ alkyl, C₈-C₉ alkyl, C₈-C₁₀ alkyl, C₈-C₁₁ alkyl, C₉-C₁₀ alkyl, C₉-C₁₁ alkyl, C₇-C₈ alkenyl, C₇-C₉ alkenyl, C₇-C₁₀ alkenyl, C₇-C₁₁ alkenyl, C₇-C₁₂ alkenyl, C₇-C₁₃ alkenyl, C₇-C₁₄ alkenyl, C₇-C₁₅ alkenyl, C₇-C₁₆ alkenyl, C₈-C₉ alkenyl, C₈-C₁₀ alkenyl, C₈-C₁₁ alkenyl, C₉-C₁₀ alkenyl, C₉-C₁₁ alkenyl, C₂-C₃ alkynyl, C₂-C₄ alkynyl, C₇-C₈ alkynyl, C₇-C₉ alkynyl, C₇-C₁₀ alkynyl, C₇-C₁₁ alkynyl, C₇-C₁₂ alkynyl, C₇-C₁₃ alkynyl, C₇-C₁₄ alkynyl, C₇-C₁₅ alkynyl, C₇-C₁₆ alkynyl, C₈-C₉ alkynyl, C₈-C₁₀ alkynyl, C₈-C₁₁ alkynyl, C₉-C₁₀ alkynyl, and/or C₉-C₁₁ alkynyl. In some embodiments, R₁ and R₂ are the same. In some embodiments, R₃ is an optionally substituted C₇-C₈ alkyl, C₇-C₉ alkyl, C₇-C₁₀ alkyl, C₇-C₁₁ alkyl, C₇-C₁₂ alkyl, C₇-C₁₃ alkyl, C₇-C₁₄ alkyl, C₇-C₁₅ alkyl, C₇-C₁₆ alkyl, C₈-C₉ alkyl, C₈-C₁₀ alkyl, C₈-C₁₁ alkyl, C₉-C₁₀ alkyl, C₉-C₁₁ alkyl, C₇-C₈ alkenyl, C₇-C₉ alkenyl, C₇-C₁₀ alkenyl, C₇-C₁₁ alkenyl, C₇-C₁₂ alkenyl, C₇-C₁₃ alkenyl, C₇-C₁₄ alkenyl, C₇-C₁₅ alkenyl, C₇-C₁₆ alkenyl, C₈-C₉ alkenyl, C₈-C₁₀ alkenyl, C₈-C₁₁ alkenyl, C₉-C₁₀ alkenyl, C₉-C₁₁ alkenyl, C₂-C₃ alkynyl, C₂-C₄ alkynyl, C₇-C₈ alkynyl, C₇-C₉ alkynyl, C₇-C₁₀ alkynyl, C₇-C₁₁ alkynyl, C₇-C₁₂ alkynyl, C₇-C₁₃ alkynyl, C₇-C₁₄ alkynyl, C₇-C₁₅ alkynyl, C₇-C₁₆ alkynyl, C₈-C₉ alkynyl, C₈-C₁₀ alkynyl, C₈-C₁₁ alkynyl, C₉-C₁₀ alkynyl, and/or C₉-C₁₁ alkynyl. In some embodiments, R₁ and R₂ are both C₈ alkyl and R₃ is C₉ alkyl. In some embodiments, n is 3, 4, 5, 6, or 7. In some embodiments, n is 4.

In embodiments, the disclosure provides reversible zwitterionic lipids, and pharmaceutical compositions comprising the reversible zwitterionic lipids, selected from the following: 3-(dioctylamino)propyl nonyl phosphate, 4-(dioctylamino)butyl nonyl phosphate, 5-(dioctylamino)pentyl nonyl phosphate, 6-(dioctylamino)hexyl nonyl phosphate, 7-(dioctylamino)heptyl nonyl phosphate, 8-(dioctylamino)octyl nonyl phosphate, 9-(dioctylamino)nonyl nonyl phosphate, 10-(dioctylamino)decyl nonyl phosphate, 11-(dioctylamino)undecyl nonyl phosphate, 12-(dioctylamino)dodecyl nonyl phosphate, 13-(dioctylamino)tridecyl nonyl phosphate, 14-(dioctylamino)tetradecyl nonyl phosphate, 15-(dioctylamino)pentadecyl nonyl phosphate, 16-(dioctylamino)hexadecyl nonyl phosphate, 17-(dioctylamino)heptadecyl nonyl phosphate, 18-(dioctylamino)octadecyl nonyl phosphate, 19-(dioctylamino)nonadecyl nonyl phosphate, and/or 20-(dioctylamino)icosyl nonyl phosphate.

In embodiments, the disclosure provides reversible zwitterionic lipids, and pharmaceutical compositions comprising the reversible zwitterionic lipids, selected from the following: 3-(dioctylamino)propyl heptyl phosphate, 3-(dinonylamino)propyl heptyl phosphate, 3-(didecylamino)propyl heptyl phosphate, 3-(diundecylamino)propyl heptyl phosphate, 3-(dioctylamino)propyl octyl phosphate, 3-(dinonylamino)propyl octyl phosphate, 3-(didecylamino)propyl octyl phosphate, 3-(diundecylamino)propyl octyl phosphate, 3-(dioctylamino)propyl nonyl phosphate, 3-(dinonylamino)propyl nonyl phosphate, 3-(didecylamino)propyl nonyl phosphate, 3-(diundecylamino)propyl nonyl phosphate, decyl (3-(dioctylamino)propyl) phosphate, decyl (3-(dinonylamino)propyl) phosphate, decyl (3-(didecylamino)propyl) phosphate, decyl (3-(diundecylamino)propyl) phosphate, 3-(dioctylamino)propyl undecyl phosphate, 3-(dinonylamino)propyl undecyl phosphate, 3-(didecylamino)propyl undecyl phosphate, 3-(diundecylamino)propyl undecyl phosphate, 3-(dioctylamino)propyl dodecyl phosphate, 3-(dinonylamino)propyl dodecyl phosphate, 3-(didecylamino)propyl dodecyl phosphate, and/or 3-(diundecylamino)propyl dodecyl phosphate, and salts and isomers thereof.

In embodiments, the disclosure provides reversible zwitterionic lipids, and pharmaceutical compositions comprising the reversible zwitterionic lipids, selected from the following: 4-(dioctylamino)butyl heptyl phosphate, 4-(dinonylamino)butyl heptyl phosphate, 4-(didecylamino)butyl heptyl phosphate, 4-(diundecylamino)butyl heptyl phosphate, 4-(dioctylamino)butyl octyl phosphate, 4-(dinonylamino)butyl octyl phosphate, 4-(didecylamino)butyl octyl phosphate, 4-(diundecylamino)butyl octyl phosphate, 4-(dioctylamino)butyl nonyl phosphate, 4-(dinonylamino)butyl nonyl phosphate, 4-(didecylamino)butyl nonyl phosphate, 4-(diundecylamino)butyl nonyl phosphate, decyl (4-(dioctylamino)butyl) phosphate, decyl (4-(dinonylamino)butyl) phosphate, decyl (4-(didecylamino)butyl) phosphate, decyl (4-(diundecylamino)butyl) phosphate, 4-(dioctylamino)butyl undecyl phosphate, 4-(dinonylamino)butyl undecyl phosphate, 4-(didecylamino)butyl undecyl phosphate, 4-(diundecylamino)butyl undecyl phosphate, 4-(dioctylamino)butyl dodecyl phosphate, 4-(dinonylamino)butyl dodecyl phosphate, 4-(didecylamino)butyl dodecyl phosphate, and/or 4-(diundecylamino)butyl dodecyl phosphate, and salts and isomers thereof.

In embodiments, the disclosure provides reversible zwitterionic lipids, and pharmaceutical compositions comprising the reversible zwitterionic lipids, selected from the following: 5-(dioctylamino)pentyl heptyl phosphate, 5-(dinonylamino)pentyl heptyl phosphate, 5-(didecylamino)pentyl heptyl phosphate, 5-(diundecylamino)pentyl heptyl phosphate, 5-(dioctylamino)pentyl octyl phosphate, 5-(dinonylamino)pentyl octyl phosphate, 5-(didecylamino)pentyl octyl phosphate, 5-(diundecylamino)pentyl octyl phosphate, 5-(dioctylamino)pentyl nonyl phosphate, 5-(dinonylamino)pentyl nonyl phosphate, 5-(didecylamino)pentyl nonyl phosphate, 5-(diundecylamino)pentyl nonyl phosphate, decyl (5-(dioctylamino)pentyl) phosphate, decyl (5-(dinonylamino)pentyl) phosphate, decyl (5-(didecylamino)pentyl) phosphate, decyl (5-(diundecylamino)pentyl) phosphate, 5-(dioctylamino)pentyl undecyl phosphate, 5-(dinonylamino)pentyl undecyl phosphate, 5-(didecylamino)pentyl undecyl phosphate, 5-(diundecylamino)pentyl undecyl phosphate, 5-(dioctylamino)pentyl dodecyl phosphate, 5-(dinonylamino)pentyl dodecyl phosphate, 5-(didecylamino)pentyl dodecyl phosphate, and/or 5-(diundecylamino)pentyl dodecyl phosphate, and salts and isomers thereof.

In embodiments, the disclosure provides reversible zwitterionic lipids, and pharmaceutical compositions comprising the reversible zwitterionic lipids, selected from the following: 6-(dioctylamino)hexyl heptyl phosphate, 6-(dinonylamino)hexyl heptyl phosphate, 6-(didecylamino)hexyl heptyl phosphate, 6-(diundecylamino)hexyl heptyl phosphate, 6-(dioctylamino)hexyl octyl phosphate, 6-(dinonylamino)hexyl octyl phosphate, 6-(didecylamino)hexyl octyl phosphate, 6-(diundecylamino)hexyl octyl phosphate, 6-(dioctylamino)hexyl nonyl phosphate, 6-(dinonylamino)hexyl nonyl phosphate, 6-(didecylamino)hexyl nonyl phosphate, 6-(diundecylamino)hexyl nonyl phosphate, decyl (6-(dioctylamino)hexyl) phosphate, decyl (6-(dinonylamino)hexyl) phosphate, decyl (6-(didecylamino)hexyl) phosphate, decyl (6-(diundecylamino)hexyl) phosphate, 6-(dioctylamino)hexyl undecyl phosphate, 6-(dinonylamino)hexyl undecyl phosphate, 6-(didecylamino)hexyl undecyl phosphate, 6-(diundecylamino)hexyl undecyl phosphate, 6-(dioctylamino)hexyl dodecyl phosphate, 6-(dinonylamino)hexyl dodecyl phosphate, 6-(didecylamino)hexyl dodecyl phosphate, and/or 6-(diundecylamino)hexyl dodecyl phosphate, and salts and isomers thereof.

In embodiments, the disclosure provides reversible zwitterionic lipids, and pharmaceutical compositions comprising the reversible zwitterionic lipids, selected from the following: 7-(dioctylamino)heptyl heptyl phosphate, 7-(dinonylamino)heptyl heptyl phosphate, 7-(didecylamino)heptyl heptyl phosphate, 7-(diundecylamino)heptyl heptyl phosphate, 7-(dioctylamino)heptyl octyl phosphate, 7-(dinonylamino)heptyl octyl phosphate, 7-(didecylamino)heptyl octyl phosphate, 7-(diundecylamino)heptyl octyl phosphate, 7-(dioctylamino)heptyl nonyl phosphate, 7-(dinonylamino)heptyl nonyl phosphate, 7-(didecylamino)heptyl nonyl phosphate, 7-(diundecylamino)heptyl nonyl phosphate, decyl (7-(dioctylamino)heptyl) phosphate, decyl (7-(dinonylamino)heptyl) phosphate, decyl (7-(didecylamino)heptyl) phosphate, decyl (7-(diundecylamino)heptyl) phosphate, 7-(dioctylamino)heptyl undecyl phosphate, 7-(dinonylamino)heptyl undecyl phosphate, 7-(didecylamino)heptyl undecyl phosphate, 7-(diundecylamino)heptyl undecyl phosphate, 7-(dioctylamino)heptyl dodecyl phosphate, 7-(dinonylamino)heptyl dodecyl phosphate, 7-(didecylamino)heptyl dodecyl phosphate, and/or 7-(diundecylamino)heptyl dodecyl phosphate, and salts and isomers thereof.

In embodiments, the disclosure provides reversible zwitterionic lipids, and pharmaceutical compositions comprising the reversible zwitterionic lipids, selected from the following: 8-(dioctylamino)octyl heptyl phosphate, 8-(dinonylamino)octyl heptyl phosphate, 8-(didecylamino)octyl heptyl phosphate, 8-(diundecylamino)octyl heptyl phosphate, 8-(dioctylamino)octyl octyl phosphate, 8-(dinonylamino)octyl octyl phosphate, 8-(didecylamino)octyl octyl phosphate, 8-(diundecylamino)octyl octyl phosphate, 8-(dioctylamino)octyl nonyl phosphate, 8-(dinonylamino)octyl nonyl phosphate, 8-(didecylamino)octyl nonyl phosphate, 8-(diundecylamino)octyl nonyl phosphate, decyl (8-(dioctylamino)octyl) phosphate, decyl (8-(dinonylamino)octyl) phosphate, decyl (8-(didecylamino)octyl) phosphate, decyl (8-(diundecylamino)octyl) phosphate, 8-(dioctylamino)octyl undecyl phosphate, 8-(dinonylamino)octyl undecyl phosphate, 8-(didecylamino)octyl undecyl phosphate, 8-(diundecylamino)octyl undecyl phosphate, 8-(dioctylamino)octyl dodecyl phosphate, 8-(dinonylamino)octyl dodecyl phosphate, 8-(didecylamino)octyl dodecyl phosphate, and/or 8-(diundecylamino)octyl dodecyl phosphate, and salts and isomers thereof.

In embodiments, the disclosure provides reversible zwitterionic lipids, and pharmaceutical compositions comprising the reversible zwitterionic lipids, selected from the group consisting of: (Z)-4-(dioctylamino)butyl non-3-en-1-yl hydrogen phosphate, 4-(dioctylamino)butyl (7-methyloctyl) hydrogen phosphate, 2-butylhexyl (4-(dioctylamino)butyl) hydrogen phosphate, (E)-4-(dioctylamino)but-2-en-1-yl nonyl hydrogen phosphate, (dioctylamino)ethynyl nonyl hydrogen phosphate, 6-(dioctylamino)hexyl nonyl hydrogen phosphate, 5-(dioctylamino)pentyl nonyl hydrogen phosphate, (Z)-4-(dioctylamino)but-2-en-1-yl nonyl hydrogen phosphate, 4-(dioctylamino)butyl (3-propylhexyl) hydrogen phosphate, and salts and isomers thereof.

In some embodiments, R₁ and/or R₂ include 1, 2, 3, 4, 5, 6, or more sites of unsaturation that correspond to, for example, cis double bonds, trans double bonds, or combinations thereof, and may be located at specific positions in one or both of the unsaturated R₁ and R₂ side-chains. For those unsaturated side-chains where a double bond is located between hydrogen atoms and alkyl or alkylene chains, the chemical notation “E” refers to the trans double bond configuration and the chemical notation “Z” refers to the cis double bond configuration. As non-limiting examples, one or both R₁ and R₂ are C₈ alkyl groups containing any combination of double bonds in the cis and/or trans configuration at one or more positions, and/or are of any structure shown in the below Examples. Similarly, as non-limiting examples, one or both R₁ and R₂ are C₁₂ alkyl groups containing any combination of double bonds which can be characterized by either the “E” chemical notation and/or the “Z” chemical notation at one or more positions in the side-chain. In some embodiments, the positions of saturation in R₁ and R₂ are the same.

In some embodiments, R₃ includes 1, 2, 3, 4, 5, 6, or more sites of unsaturation that correspond to, for example, cis double bonds, trans double bonds, or combinations thereof, and may be located at specific positions in one or both of the unsaturated R₁ and R₂ side-chains. For those unsaturated side-chains where a double bond is located between hydrogen atoms and alkyl or alkylene chains, the chemical notation “E” refers to the trans double bond configuration and the chemical notation “Z” refers to the cis double bond configuration. As non-limiting examples, one or both R₃ is C₉ alkyl groups containing any combination of double bonds in the cis and/or trans configuration at one or more positions. As another non-limiting example, R₃ may have the following structure:

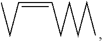

or any R₃ structure shown in the below Examples.

In some embodiments, R₁, R₂, and R₃ are independently an alkenyl selected from the group consisting of hept-1-ene, hept-2-ene, hept-3-ene, oct-1-ene, oct-2-ene, oct-3-ene, oct-4-ene, non-1-ene, non-2-ene, non-3-ene, non-4-ene, non-5-ene, dec-1-ene, dec-2-ene, dec-3-ene, dec-4-ene, dec-5-ene, dec-6-ene, undec-1-ene, undec-2-ene, undec-3-ene, undec-4-ene, undec-5-ene, undec-6-ene, undec-7-ene, dodec-1-ene, dodec-2-ene, dodec-3-ene, dodec-4-ene, dodec-5-ene, dodec-6-ene, and dodec-8-ene.

In some embodiments, R₁, R₂, and R₃ are independently an alkynyl selected from the group consisting of hept-1-yne, hept-2-yne, hept-3-yne, oct-1-yne, oct-2-yne, oct-3-yne, oct-4-yne, non-1-yne, non-2-yne, non-3-yne, non-4-yne, non-5-yne, dec-1-yne, dec-2-yne, dec-3-yne, dec-4-yne, dec-5-yne, dec-6-yne, undec-1-yne, undec-2-yne, undec-3-yne, undec-4-yne, undec-5-yne, undec-6-yne, undec-7-yne, dodec-1-yne, dodec-2-yne, dodec-3-yne, dodec-4-yne, dodec-5-yne, dodec-6-yne, and dodec-8-yne.

In some embodiments, the linker connecting the phosphate group and the amine group may include 1, 2, 3, 4, 5, 6, or more sites of unsaturation that correspond to, for example, cis double bonds, trans double bonds, or combinations thereof, and/or one or more triple bonds and may be located at specific positions within the linker. Exemplary embodiments include, but are not limited to, (E)-4-(dioctylamino)but-2-en-1-yl nonyl hydrogen phosphate and (dioctylamino)ethynyl nonyl hydrogen phosphate, having the following structures:

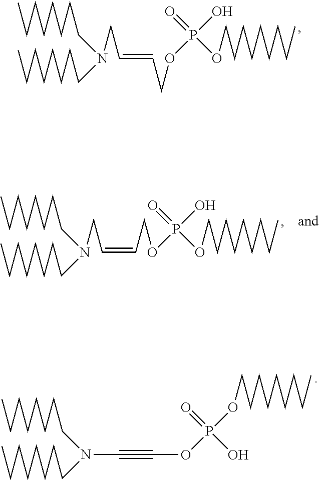

In embodiments, the present disclosure provides a reversible zwitterionic lipid of Formula II having the following structure:

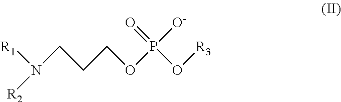

or salts thereof, wherein:
R₁ and R₂ are either the same or different and are independently and optionally substituted C₇-C₂₂ alkyl, C₇-C₂₂ alkenyl, or C₇-C₂₂ alkynyl, optionally R₁, R₂, or R₁ and R₂ are an optionally substituted heterocycle or R₁ and R₂ may join to form an optionally substituted heterocycle; and
R₃ is optionally substituted C₃-C₂₂ alkyl, C₃-C₂₂ alkenyl, or C₃-C₂₂ alkynyl.

In some embodiments, R₁ and R₂ are each independently C₇-C₈ alkyl, C₇-C₉ alkyl, C₇-C₁₀ alkyl, C₇-C₁₁ alkyl, C₇-C₁₂ alkyl, C₇-C₁₃ alkyl, C₇-C₁₄ alkyl, C₇-C₁₅ alkyl, C₇-C₁₆ alkyl, C₈-C₉ alkyl, C₈-C₁₀ alkyl, C₈-C₁₁ alkyl, C₉-C₁₀ alkyl, C₉-C₁₁ alkyl, C₇-C₈ alkenyl, C₇-C₉ alkenyl, C₇-C₁₀ alkenyl, C₇-C₁₁ alkenyl, C₇-C₁₂ alkenyl, C₇-C₁₃ alkenyl, C₇-C₁₄ alkenyl, C₇-C₁₅ alkenyl, C₇-C₁₆ alkenyl, C₈-C₉ alkenyl, C₈-C₁₀ alkenyl, C₈-C₁₁ alkenyl, C₉-C₁₀ alkenyl, C₉-C₁₁ alkenyl, C₂-C₃ alkynyl, C₂-C₄ alkynyl, C₇-C₈ alkynyl, C₇-C₉ alkynyl, C₇-C₁₀ alkynyl, C₇-C₁₁ alkynyl, C₇-C₁₂ alkynyl, C₇-C₁₃ alkynyl, C₇-C₁₄ alkynyl, C₇-C₁₅ alkynyl, C₇-C₁₆ alkynyl, C₈-C₉ alkynyl, C₈-C₁₀ alkynyl, C₈-C₁₁ alkynyl, C₉-C₁₀ alkynyl, and/or C₉-C₁₁ alkynyl. In some embodiments, R₁ and R₂ are the same.

In some embodiments, R₃ is an optionally substituted C₇-C₈ alkyl, C₇-C₉ alkyl, C₇-C₁₀ alkyl, C₇-C₁₁ alkyl, C₇-C₁₂ alkyl, C₇-C₁₃ alkyl, C₇-C₁₄ alkyl, C₇-C₁₅ alkyl, C₇-C₁₆ alkyl, C₈-C₉ alkyl, C₈-C₁₀ alkyl, C₈-C₁₁ alkyl, C₉-C₁₀ alkyl, C₉-C₁₁ alkyl, C₇-C₈ alkenyl, C₇-C₉ alkenyl, C₇-C₁₀ alkenyl, C₇-C₁₁ alkenyl, C₇-C₁₂ alkenyl, C₇-C₁₃ alkenyl, C₇-C₁₄ alkenyl, C₇-C₁₅ alkenyl, C₇-C₁₆ alkenyl, C₈-C₉ alkenyl, C₈-C₁₀ alkenyl, C₈-C₁₁ alkenyl, C₉-C₁₀ alkenyl, C₉-C₁₁ alkenyl, C₂-C₃ alkynyl, C₂-C₄ alkynyl, C₇-C₈ alkynyl, C₇-C₉ alkynyl, C₇-C₁₀ alkynyl, C₇-C₁₁ alkynyl, C₇-C₁₂ alkynyl, C₇-C₁₃ alkynyl, C₇-C₁₄ alkynyl, C₇-C₁₅ alkynyl, C₇-C₁₆ alkynyl, C₈-C₉ alkynyl, C₈-C₁₀ alkynyl, C₈-C₁₁ alkynyl, C₉-C₁₀ alkynyl, and/or C₉-C₁₁ alkynyl. In some embodiments, R₁ and R₂ are both C₈ alkyl and R₃ is C₉ alkyl. In some embodiments, n is 3, 4, 5, 6, or 7. In some embodiments, n is 3 or 4.

In some embodiments, R₁ and/or R₂ include 1, 2, 3, 4, 5, 6, or more sites of unsaturation that correspond to, for example, cis double bonds, trans double bonds, or combinations thereof, and may be located at specific positions in one or both of the unsaturated R₁ and R₂ side-chains. For those unsaturated side-chains where a double bond is located between hydrogen atoms and alkyl or alkylene chains, the chemical notation “E” refers to the trans double bond configuration and the chemical notation “Z” refers to the cis double bond configuration. As non-limiting examples, one or both R₁ and R₂ are C₈ alkyl groups containing any combination of double bonds in the cis and/or trans configuration at one or more positions. Similarly, as non-limiting examples, one or both R₁ and R₂ are C₁₂ alkyl groups containing any combination of double bonds which can be characterized by either the “E” chemical notation and/or the “Z” chemical notation at one or more positions in the side-chain. In some embodiments, the positions of saturation in R₁ and R₂ are the same.

In some embodiments, R₃ includes 1, 2, 3, 4, 5, 6, or more sites of unsaturation that correspond to, for example, cis double bonds, trans double bonds, or combinations thereof, and may be located at specific positions in one or both of the unsaturated R₁ and R₂ side-chains. For those unsaturated side-chains where a double bond is located between hydrogen atoms and alkyl or alkylene chains, the chemical notation “E” refers to the trans double bond configuration and the chemical notation “Z” refers to the cis double bond configuration. As non-limiting examples, one or both R₃ is C₉ alkyl groups containing any combination of double bonds in the cis and/or trans configuration at one or more positions.

In some embodiments, the linker connecting the phosphate group and the amine group may include 1, 2, 3, 4, 5, 6, or more sites of unsaturation that correspond to, for example, cis double bonds, trans double bonds, or combinations thereof, and/or one or more triple bonds and may be located at specific positions within the linker.

In embodiments, the present disclosure provides a lipid of any of the following structures:

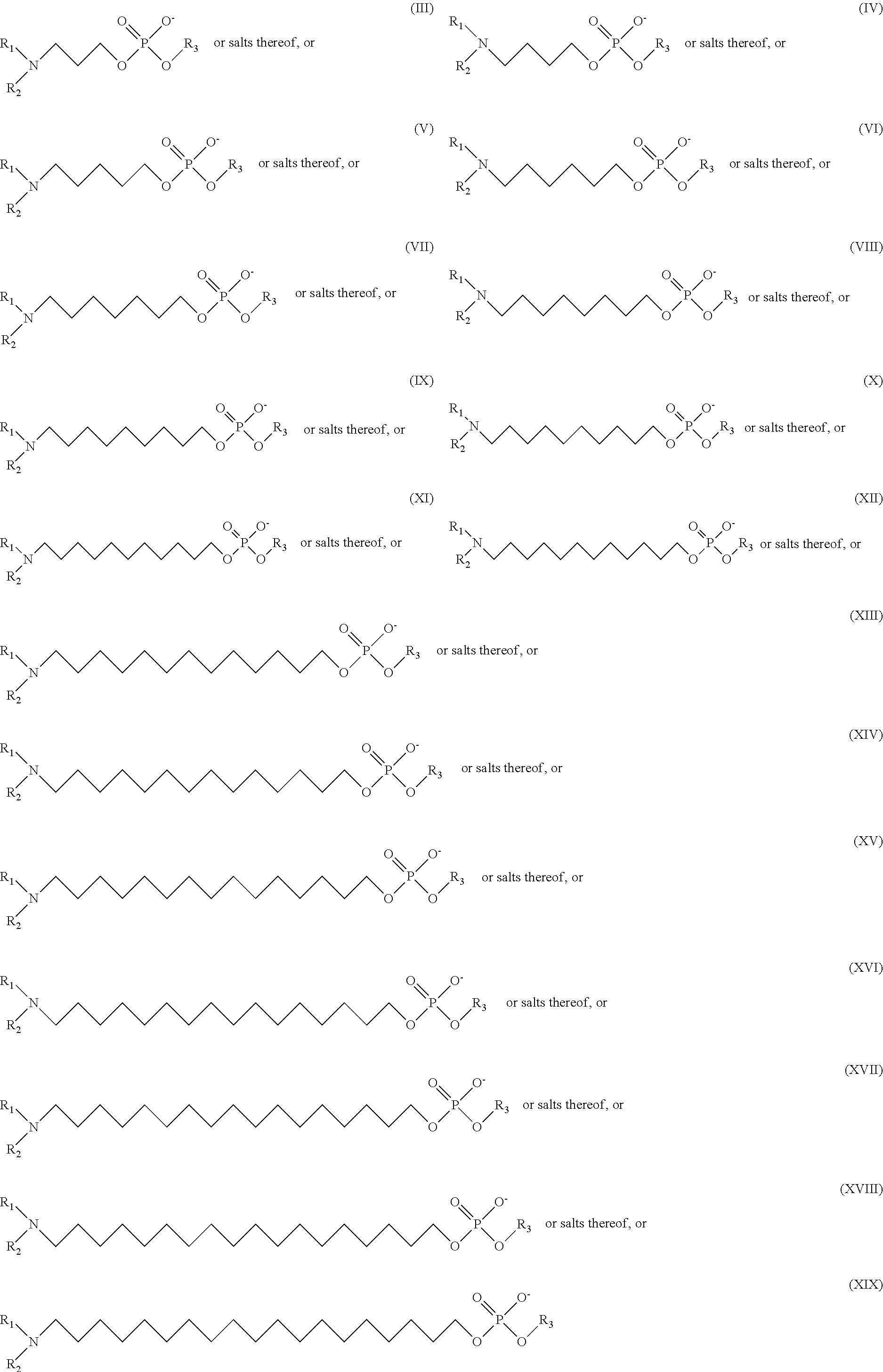

or salts thereof, wherein:
R₁ and R₂ are either the same or different and are independently and optionally substituted C₇-C₂₂ alkyl, C₇-C₂₂ alkenyl, or C₇-C₂₂ alkynyl, optionally R₁, R₂, or R₁ and R₂ are an optionally substituted heterocycle or R₁ and R₂ may join to form an optionally substituted heterocycle; and
R₃ is optionally substituted C₃-C₂₂ alkyl, C₃-C₂₂ alkenyl, or C₃-C₂₂ alkynyl.

In some embodiments, R₁ and R₂ are each independently C₇-C₈ alkyl, C₇-C₉ alkyl, C₇-C₁₀ alkyl, C₇-C₁₁ alkyl, C₇-C₁₂ alkyl, C₇-C₁₃ alkyl, C₇-C₁₄ alkyl, C₇-C₁₅ alkyl, C₇-C₁₆ alkyl, C₈-C₉ alkyl, C₈-C₁₀ alkyl, C₈-C₁₁ alkyl, C₉-C₁₀ alkyl, C₉-C₁₁ alkyl, C₁₀-C₁₁ alkyl, C₁₀-C₁₂ alkyl, C₁₀-C₁₃ alkyl, C₁₀-C₁₄ alkyl, C₇-C₈ alkenyl, C₇-C₉ alkenyl, C₇-C₁₀ alkenyl, C₇-C₁₁ alkenyl, C₇-C₁₂ alkenyl, C₇-C₁₃ alkenyl, C₇-C₁₄ alkenyl, C₇-C₁₅ alkenyl, C₇-C₁₆ alkenyl, C₈-C₉ alkenyl, C₈-C₁₀ alkenyl, C₈-C₁₁ alkenyl, C₉-C₁₀ alkenyl, C₉-C₁₁ alkenyl, C₂-C₃ alkynyl, C₂-C₄ alkynyl, C₇-C₈ alkynyl, C₇-C₉ alkynyl, C₇-C₁₀ alkynyl, C₇-C₁₁ alkynyl, C₇-C₁₂ alkynyl, C₇-C₁₃ alkynyl, C₇-C₁₄ alkynyl, C₇-C₁₅ alkynyl, C₇-C₁₆ alkynyl, C₈-C₉ alkynyl, C₈-C₁₀ alkynyl, C₈-C₁₁ alkynyl, C₉-C₁₀ alkynyl, and/or C₉-C₁₁ alkynyl. In some embodiments, R₁ and R₂ are the same.

In some embodiments, R₃ is an optionally substituted C₇-C₈ alkyl, C₇-C₉ alkyl, C₇-C₁₀ alkyl, C₇-C₁₁ alkyl, C₇-C₁₂ alkyl, C₇-C₁₃ alkyl, C₇-C₁₄ alkyl, C₇-C₁₅ alkyl, C₇-C₁₆ alkyl, C₈-C₉ alkyl, C₈-C₁₀ alkyl, C₈-C₁₁ alkyl, C₉-C₁₀ alkyl, C₉-C₁₁ alkyl, C₁₀-C₁₁ alkyl, C₁₀-C₁₂ alkyl, C₁₀-C₁₃ alkyl, C₁₀-C₁₄ alkyl, C₇-C₈ alkenyl, C₇-C₉ alkenyl, C₇-C₁₀ alkenyl, C₇-C₁₁ alkenyl, C₇-C₁₂ alkenyl, C₇-C₁₃ alkenyl, C₇-C₁₄ alkenyl, C₇-C₁₅ alkenyl, C₇-C₁₆ alkenyl, C₈-C₉ alkenyl, C₈-C₁₀ alkenyl, C₈-C₁₁ alkenyl, C₉-C₁₀ alkenyl, C₉-C₁₁ alkenyl, C₂-C₃ alkynyl, C₂-C₄ alkynyl, C₇-C₈ alkynyl, C₇-C₉ alkynyl, C₇-C₁₀ alkynyl, C₇-C₁₁ alkynyl, C₇-C₁₂ alkynyl, C₇-C₁₃ alkynyl, C₇-C₁₄ alkynyl, C₇-C₁₅ alkynyl, C₇-C₁₆ alkynyl, C₈-C₉ alkynyl, C₈-C₁₀ alkynyl, C₈-C₁₁ alkynyl, C₉-C₁₀ alkynyl, and/or C₉-C₁₁ alkynyl. In some embodiments, R₁ and R₂ are both C₈ alkyl and R₃ is C₉ alkyl.

In some embodiments, R₁ and/or R₂ include 1, 2, 3, 4, 5, 6, or more sites of unsaturation that correspond to, for example, cis double bonds, trans double bonds, or combinations thereof, and may be located at specific positions in one or both of the unsaturated R₁ and R₂ side-chains. For those unsaturated side-chains where a double bond is located between hydrogen atoms and alkyl or alkylene chains, the chemical notation “E” refers to the trans double bond configuration and the chemical notation “Z” refers to the cis double bond configuration. As non-limiting examples, one or both R₁ and R₂ are C₈ alkyl groups containing any combination of double bonds in the cis and/or trans configuration at one or more positions. Similarly, as non-limiting examples, one or both R₁ and R₂ are C₁₂ alkyl groups containing any combination of double bonds which can be characterized by either the “E” chemical notation and/or the “Z” chemical notation at one or more positions in the side-chain. In some embodiments, the positions of saturation in R₁ and R₂ are the same.

In some embodiments, R₃ includes 1, 2, 3, 4, 5, 6, or more sites of unsaturation that correspond to, for example, cis double bonds, trans double bonds, or combinations thereof, and may be located at specific positions in one or both of the unsaturated R₁ and R₂ side-chains. For those unsaturated side-chains where a double bond is located between hydrogen atoms and alkyl or alkylene chains, the chemical notation “E” refers to the trans double bond configuration and the chemical notation “Z” refers to the cis double bond configuration. As non-limiting examples, one or both R₃ is C₉ alkyl groups containing any combination of double bonds in the cis and/or trans configuration at one or more positions.

In some embodiments, R₁, R₂, and R₃ are independently an alkenyl selected from the group consisting of hept-1-ene, hept-2-ene, hept-3-ene, oct-1-ene, oct-2-ene, oct-3-ene, oct-4-ene, non-1-ene, non-2-ene, non-3-ene, non-4-ene, non-5-ene, dec-1-ene, dec-2-ene, dec-3-ene, dec-4-ene, dec-5-ene, dec-6-ene, undec-1-ene, undec-2-ene, undec-3-ene, undec-4-ene, undec-5-ene, undec-6-ene, undec-7-ene, dodec-1-ene, dodec-2-ene, dodec-3-ene, dodec-4-ene, dodec-5-ene, dodec-6-ene, and dodec-8-ene.

In some embodiments, R₁, R₂, and R₃ are independently an alkynyl selected from the group consisting of hept-1-yne, hept-2-yne, hept-3-yne, oct-1-yne, oct-2-yne, oct-3-yne, oct-4-yne, non-1-yne, non-2-yne, non-3-yne, non-4-yne, non-5-yne, dec-1-yne, dec-2-yne, dec-3-yne, dec-4-yne, dec-5-yne, dec-6-yne, undec-1-yne, undec-2-yne, undec-3-yne, undec-4-yne, undec-5-yne, undec-6-yne, undec-7-yne, dodec-1-yne, dodec-2-yne, dodec-3-yne, dodec-4-yne, dodec-5-yne, dodec-6-yne, and dodec-8-yne.

In some embodiments, the linker connecting the phosphate group and the amine group may include 1, 2, 3, 4, 5, 6, or more sites of unsaturation that correspond to, for example, cis double bonds, trans double bonds, or combinations thereof, and/or one or more triple bonds and may be located at specific positions within the linker.

In some embodiments, the present disclosure provides a reversible zwitterionic lipid selected from the following groups:

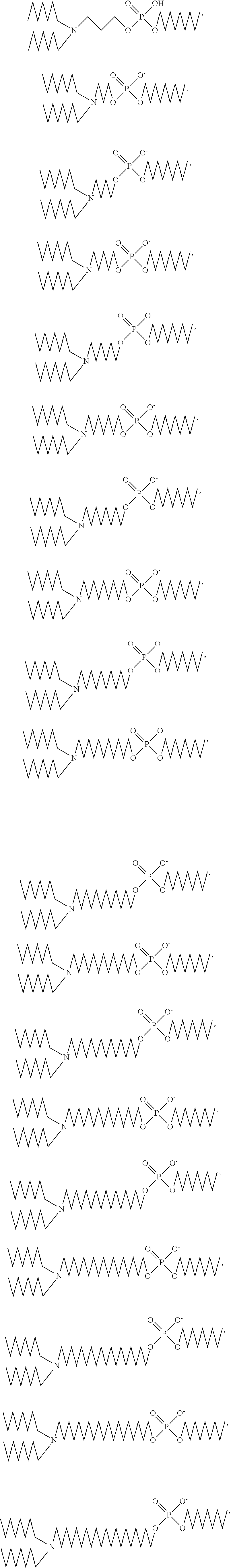

and salts and isomers thereof.

In some embodiments, the present disclosure provides a reversible zwitterionic lipid selected from the following groups:

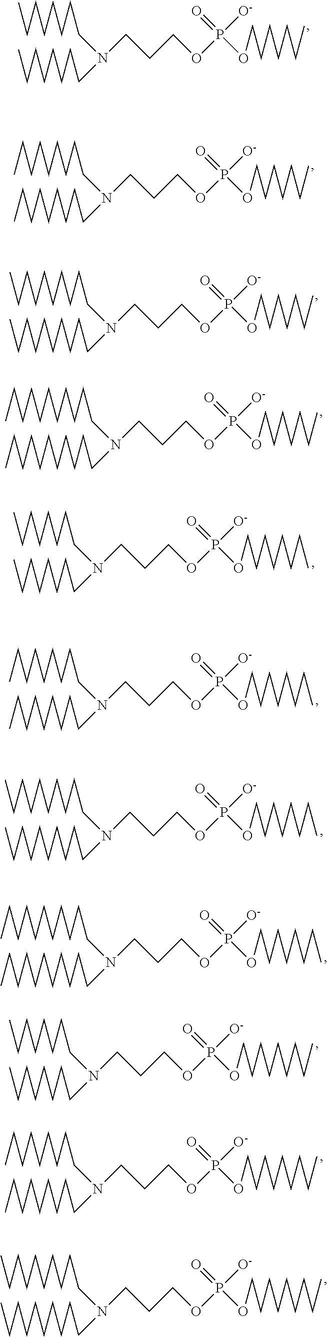

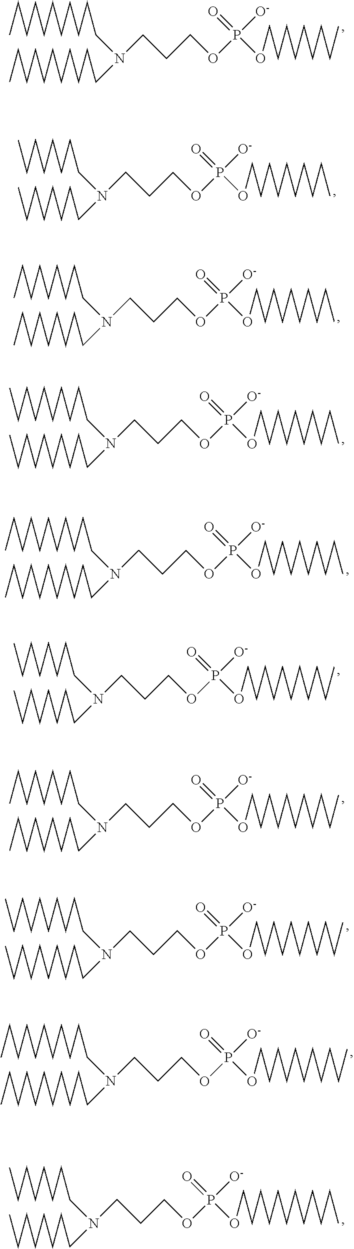

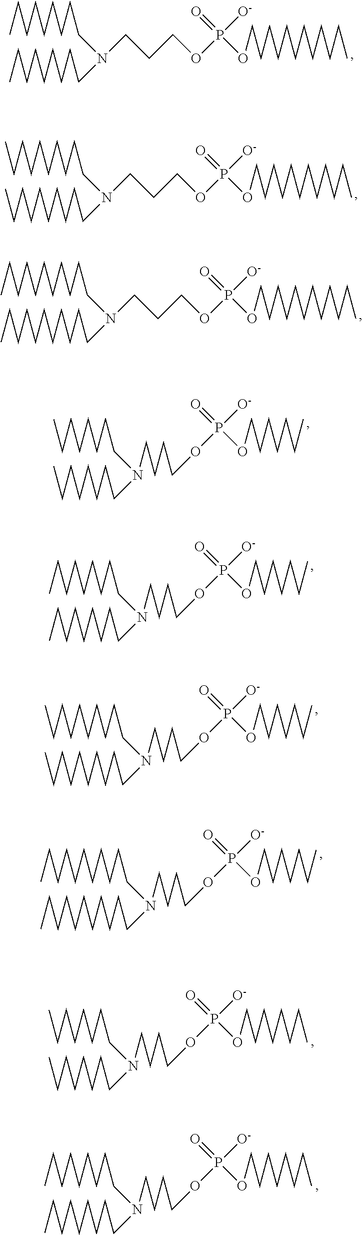

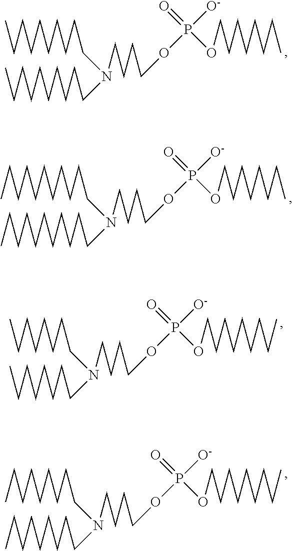

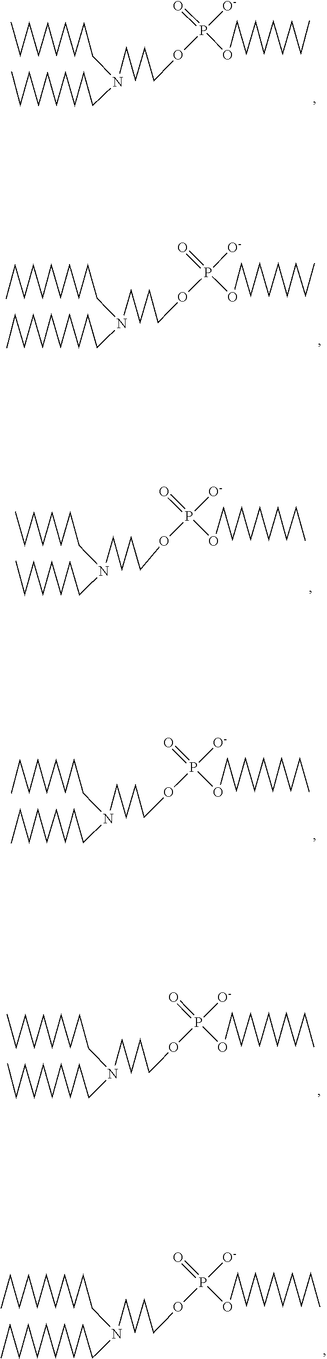

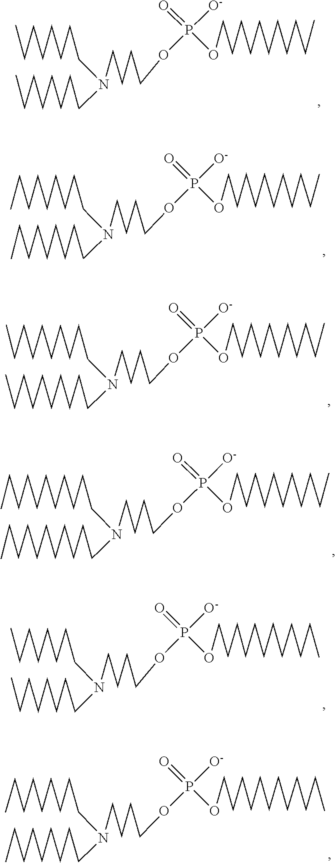

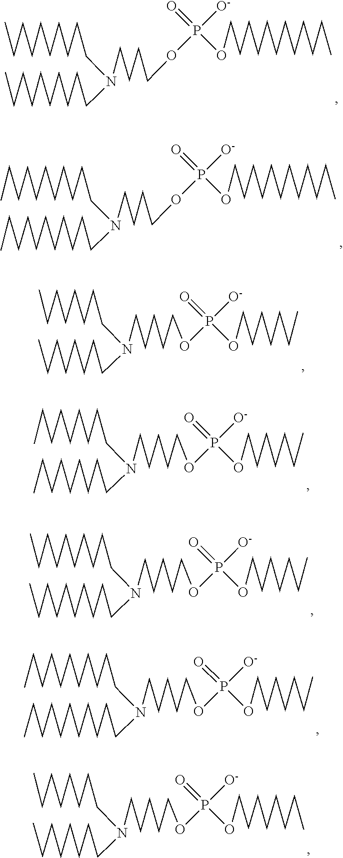

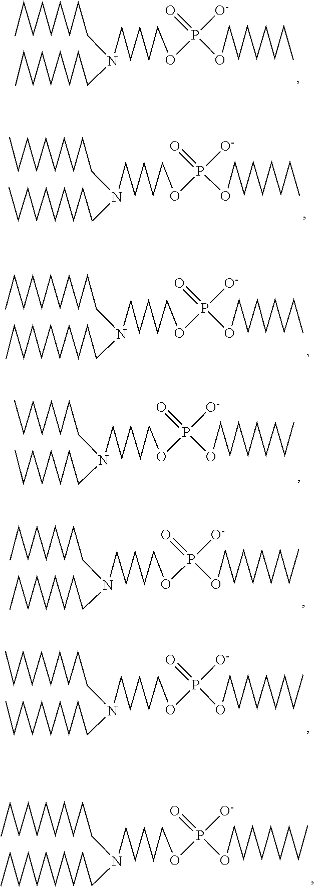

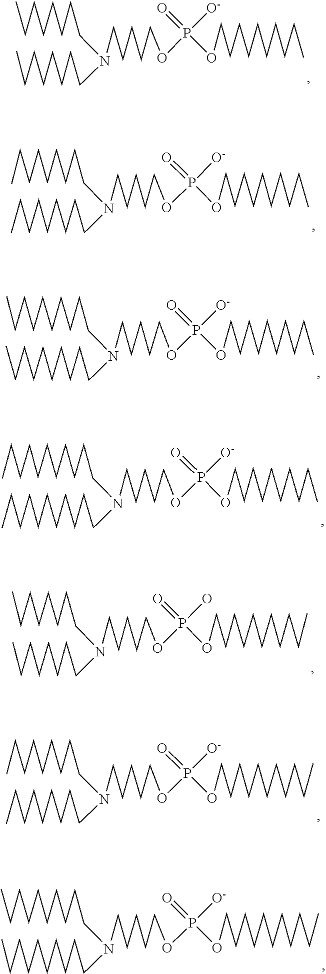

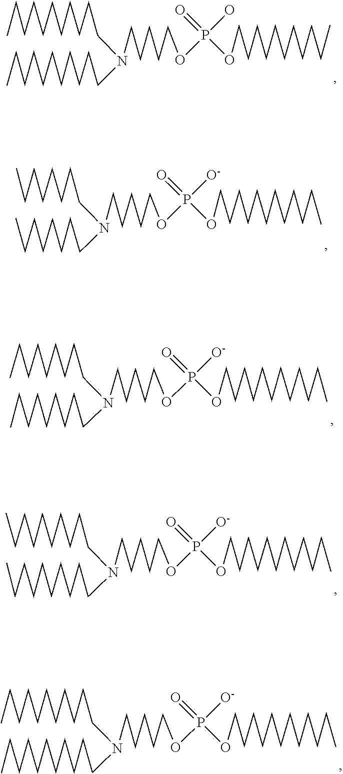

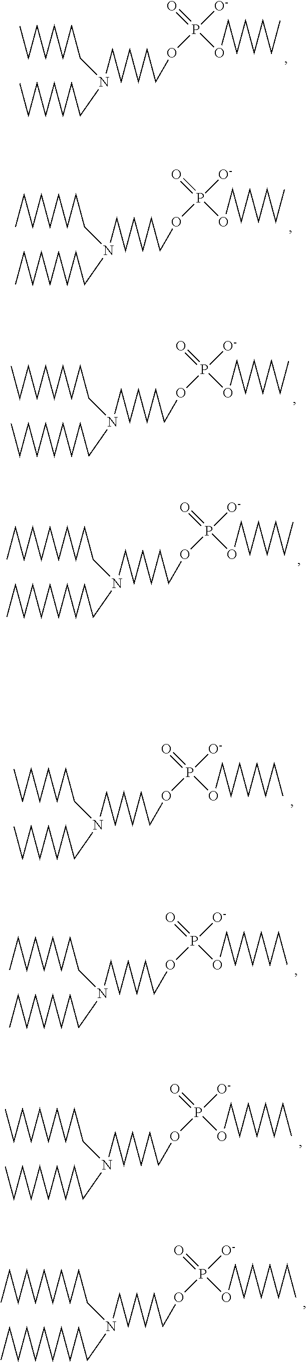

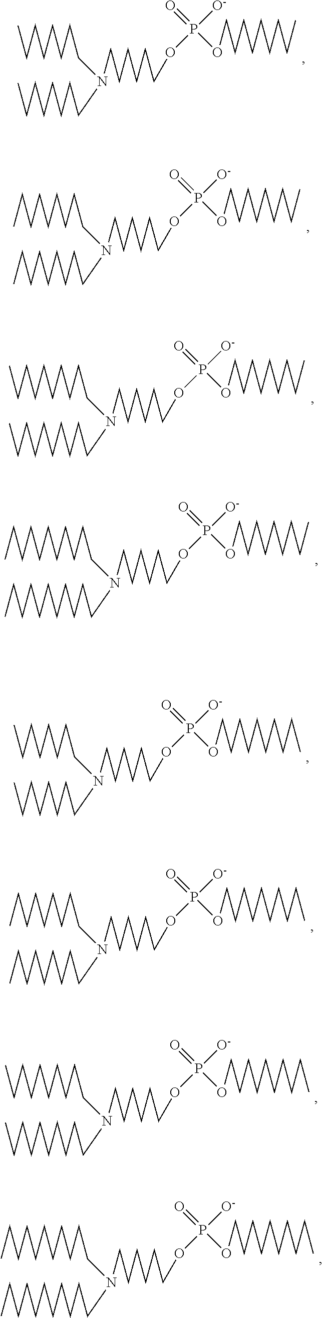

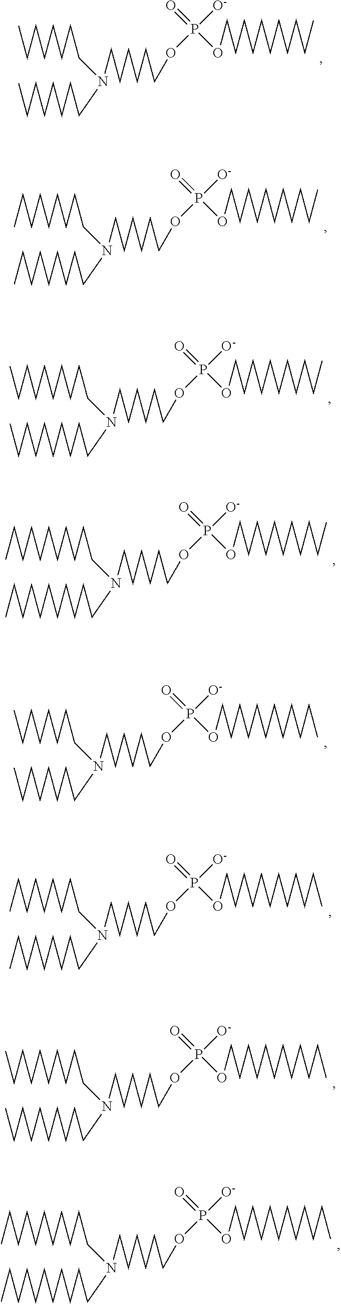

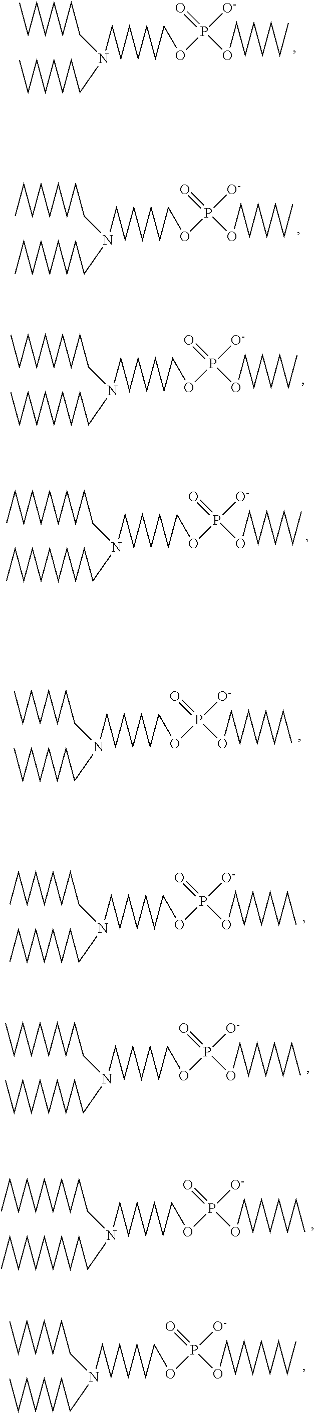

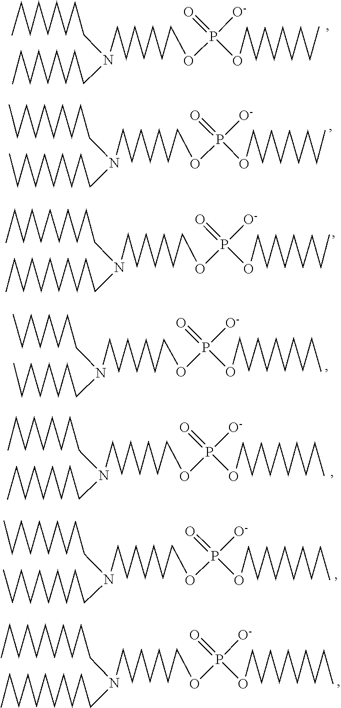

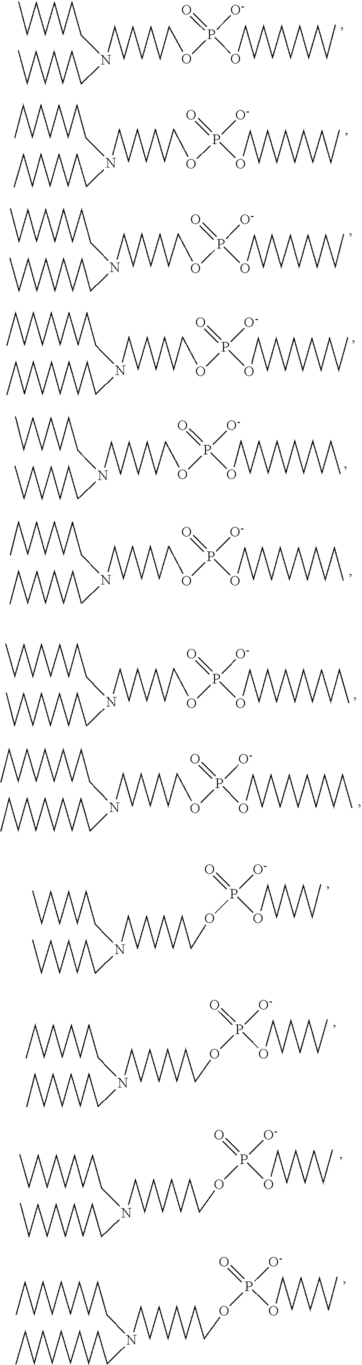

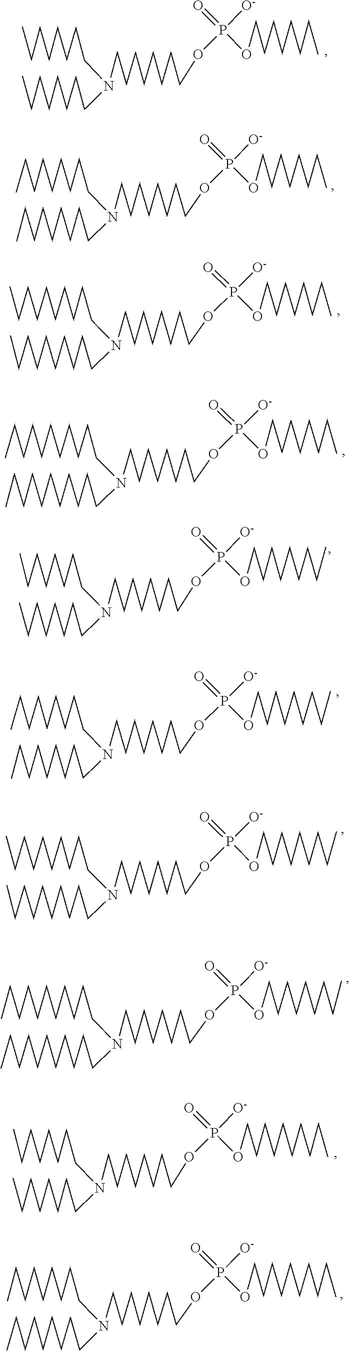

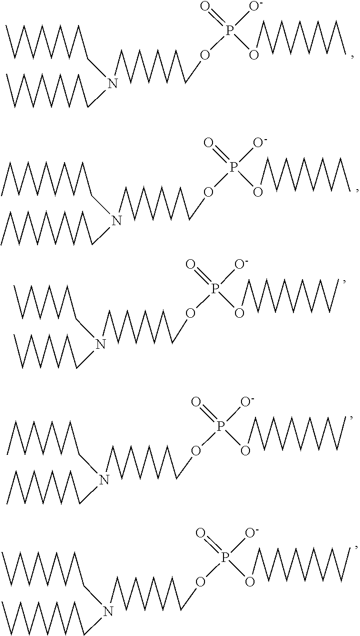

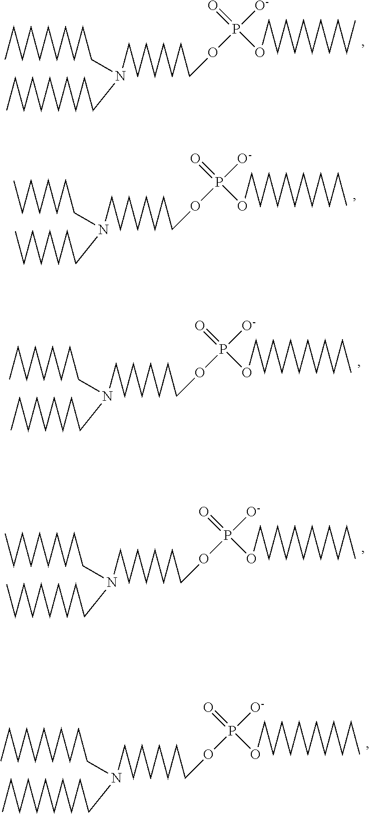

and salts and isomers thereof.

In some embodiments, the present disclosure provides a reversible zwitterionic lipid selected from the following groups:

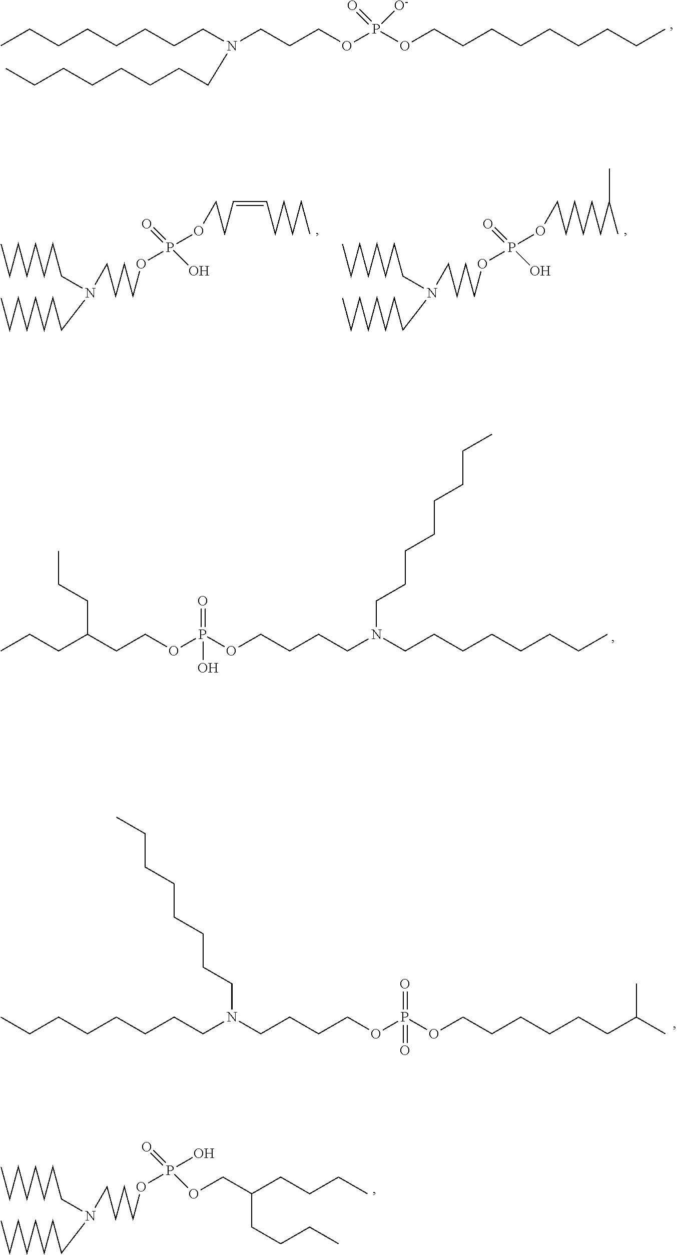

and salts and isomers thereof.

In some embodiments, the disclosure provides a reversible zwitterionic lipid selected from the group consisting of:

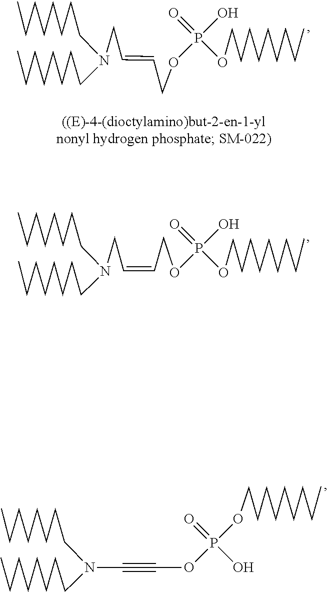

and salts and isomers thereof.

The compounds described herein may be prepared by known organic synthesis techniques, including the methods described in the below Examples.

### Lipid-Based Compositions

The techniques herein provide lipid-based compositions (e.g., LNPs and the like) comprising one or more of the reversible zwitterionic lipids or salts thereof described herein. In some embodiments, the lipid-based compositions of the disclosure further comprise one or more non-cationic lipids. In some embodiments, the lipid-based compositions further comprise one or more conjugated lipids capable of reducing or inhibiting particle aggregation. In some embodiments, the lipid-based compositions further comprise one or more active agents or therapeutic agents such as, for example, nucleic acids (e.g., siRNA, ASO, tRNA, miRNA, mRNA, DNA, and the like), proteins, peptides, and other macromolecules.

As disclosed herein, lipid-based compositions include, but are not limited to, lipid nanoparticles, lipid vesicles (e.g., liposomes), and the like. As used herein, a lipid vesicle may include a structure having lipid-containing membranes enclosing an aqueous interior. In some embodiments, lipid-based compositions comprising one or more of the reversible zwitterionic lipids described herein may be used to encapsulate therapeutic agents such as, for example, nucleic acids, within the lipid vesicles. In some embodiments, lipid vesicles comprising one or more of the reversible zwitterionic lipids described herein may be complexed with nucleic acids.

The lipid-based compositions of the disclosure typically comprise a therapeutic agent, a reversible zwitterionic lipid, a non-cationic lipid, and a conjugated lipid (e.g., a polyethylene glycol (PEG)-lipid) that inhibits aggregation of particles. In some embodiments, the therapeutic agent is fully encapsulated within the lipid portion of the lipid-based compositions such that the therapeutic agent is resistant to enzymatic degradation, e.g., by a nuclease or protease. In some embodiments, the lipid-based compositions described herein are substantially non-toxic to mammals such as humans.

It is contemplated within the scope of the disclosure that the lipid-based compositions described herein typically have a mean diameter of from about 30 nm to about 250 nm, from about 40 nm to about 200 nm, from about 50 nm to about 150 nm, from about 60 nm to about 130 nm, from about 70 nm to about 110 nm, or from about 70 to about 90 nm. in some embodiments, the lipid-based compositions disclosed herein have a lipid:therapeutic agent (e.g., lipid:nucleic acid) ratio (mass/mass ratio) of from about 1:1 to about 1000:1, from about 1:1 to about 500:1, from about 2:1 to about 250:1, from about 3:1 to about 200:1, from about 5:1 to about 150:1, from about 5:1 to about 100:1, from about 5:1 to about 50:1, from about 5:1 to about 25:1, from about 5:1 to about 20:1, from about 5:1 to about 10:1, or from about 6:1 to about 9:1. Alternatively, the lipid-based compositions disclosed herein have a lipid:therapeutic agent (e.g., lipid:nucleic acid) ratio (mole/mole ratio) of from about 1:1 to about 30:1, from about 2:1 to about 20:1, from about 2:1 to about 15:1, from about 3:1 to about 10:1, from about 4:1 to about 9:1, from about 5:1 to about 8:1, or from about 6:1 to about 8:1.

In some embodiments, the lipid-based compositions of the disclosure are nucleic acid-lipid particles that include an interfering RNA (e.g., dsRNA such as siRNA, Dicer-substrate dsRNA, shRNA, aiRNA, and/or miRNA), an ionizable lipid (e.g., one or more lipids of Formulas I-XIX or salts thereof as set forth herein), a non-cationic lipid (e.g., mixtures of one or more phospholipids and cholesterol), and a conjugated lipid that inhibits aggregation of the particles (e.g., one or more PEG-lipid conjugates). The nucleic acid-lipid particle may comprise at least 1, 2, 3, 4, 5, 6, 7, 8, 9, 10, or more unmodified and/or modified interfering RNA molecules (e.g., siRNA). Nucleic acid-lipid particles and their method of preparation are described in, e.g., U.S. Pat. Nos. 5,753,613; 5,785,992; 5,705,385; 5,976,567; 5,981,501; 6,110,745; and 6,320,017; and PCT Publication No. WO 96/40964, the disclosures of which are each herein incorporated by reference in their entirety for all purposes.

In the nucleic acid-lipid particles disclosed herein, the nucleic acid may be fully encapsulated within the lipid portion of the particle, thereby protecting the nucleic acid from nuclease degradation. In preferred embodiments, a nucleic acid-lipid particle comprising a nucleic acid such as an interfering RNA may be fully encapsulated within the lipid portion of the particle, thereby protecting the nucleic acid from nuclease degradation. In some embodiments, the nucleic acid may be complexed with the lipid portion of the particle. It is contemplated within the scope of the disclosure that the lipid-based compositions disclosed herein are substantially non-toxic to mammals such as humans.

As used herein, the term “fully encapsulated” indicates that the nucleic acid in the nucleic acid-lipid particle is not significantly degraded after exposure to serum or a nuclease assay that would significantly degrade free DNA or RNA. In a fully encapsulated system, preferably less than about 25% of the nucleic acid in the particle is degraded in a treatment that would normally degrade 100% of free nucleic acid, more preferably less than about 10%, and most preferably less than about 5% of the nucleic acid in the particle is degraded.

In some embodiments, the present disclosure provides a nucleic acid-lipid particle composition comprising a plurality of nucleic acid-lipid particles.

In some instances, the nucleic acid-lipid particle composition comprises nucleic acid that is fully encapsulated within the lipid portion of the particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the particles have the nucleic acid encapsulated therein.

The techniques herein provide that the proportions of the components within the lipid-based compositions may be varied and the delivery efficiency of a particular formulation can be measured using, e.g., an endosomal release parameter (ERP) assay. It is contemplated within the scope of the disclosure that the lipid-based compositions disclosed herein have increased delivery efficiency due to enhanced endosomal release caused, at least in part, by the novel reversible zwitterionic lipids disclosed herein.

According to the techniques herein, any one or more of the novel reversible zwitterionic lipids of Formulas I-XIX may be used in the lipid-based compositions disclosed herein, either alone or in combination with one or more other cationic lipid species or non-cationic lipid species.

Other obligate cationic lipids or salts thereof and/or ionizable lipids or salts thereof may also be included in the lipid-based compositions of the present disclosure

In some embodiments, the reversible zwitterionic lipids disclosed herein comprise from about 40 mol % to about 90 mol %, from about 40 mol % to about 85 mol %, from about 40 mol % to about 80 mol %, from about 40 mol % to about 75 mol %, from about 40 mol % to about 70 mol %, from about 40 mol % to about 65 mol %, from about 40 mol % to about 60 mol %, from about 40 mol % to about 55 mol %, from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, from about 50 mol % to about 60 mol % of the total lipid present in the particle.

In some embodiments, the reversible zwitterionic lipids disclosed herein comprise from about 50 mol % to about 58 mol %, from about 51 mol % to about 59 mol %, from about 51 mol % to about 58 mol %, from about 51 mol % to about 57 mol %, from about 52 mol % to about 58 mol %, from about 52 mol % to about 57 mol %, from about 52 mol % to about 56 mol %, or from about 53 mol % to about 55 mol % of the total lipid present in the particle. In some embodiments, the cationic lipid comprises about 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, or 65 mol % (or any fraction thereof or range therein) of the total lipid present in the particle. In some embodiments, the ionizable lipid comprises at least about 66, 67, 68, 69, 70, 71, 72, 73, 74, 75, 76, 77, 78, 79, 80, 81, 82, 83, 84, 85, 86, 87, 88, 89, or 90 mol % of the total lipid present in the particle.

In some embodiments, the reversible zwitterionic lipids disclosed herein comprises from about 2 mol % to about 60 mol %, from about 5 mol % to about 50 mol %, from about 10 mol % to about 50 mol %, from about 20 mol % to about 50 mol %, from about 20 mol % to about 40 mol %, from about 30 mol % to about 40 mol %, or about 40 mol % of the total lipid present in the particle.

One of skill in the art will appreciate that the percentage of reversible zwitterionic lipid present in the lipid-based compositions of the disclosure is a target amount, and that the actual amount of cationic lipid present in the formulation may vary, for example, by about ±5 mol %.

The lipid-based compositions disclosed herein may also include a variety of non-cationic lipids including, but not limited to, phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl.

Other examples of non-cationic lipids may include, but are not limited to, sterols such as cholesterol and derivatives thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In preferred embodiments, the cholesterol derivative is a polar analogue such as cholesteryl-(4′-hydroxy)-butyl ether.

In some embodiments, the non-cationic lipid comprises from about 10 mol % to about 60 mol %, from about 20 mol % to about 55 mol %, from about 20 mol % to about 45 mol %, from about 20 mol % to about 40 mol %, from about 25 mol % to about 50 mol %, from about 25 mol % to about 45 mol %, from about 30 mol % to about 50 mol %, from about 30 mol % to about 45 mol %, from about 30 mol % to about 40 mol %, from about 35 mol % to about 45 mol %, from about 37 mol % to about 42 mol %, or about 35 mol %, 36 mol %, 37 mol %, 38 mol %, 39 mol %, 40 mol %, 41 mol %, 42 mol %, 43 mol %, 44 mol %, or 45 mol % (or any fraction thereof or range therein) of the total lipid present in the particle.

As discussed above with respect to cationic lipids, one of skill in the art will also appreciate that the percentage of non-cationic lipid present in the lipid particles of the disclosure is a target amount, and that the actual amount of non-cationic lipid present in the formulation may vary, for example, by ±5 mol %.

Lipid nanoparticles of any size may be used according to the instant disclosure. In certain embodiments of the instant disclosure, lipid nanoparticles have a size ranging from about 0.02 microns to about 0.4 microns, between about 0.05 and about 0.2 microns, or between 0.07 and 0.12 microns in diameter.

In some embodiments, the particles of the instant disclosure may include neutral lipids, for example, diacylphosphatidylcholine, diacylphosphatidylethanolamine, ceramide, sphingomyelin, cephalin, cholesterol, cerebrosides and diacylglycerols. In other embodiments, LNPs may include anionic lipids, including but not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids. In some aspects, the non-cationic lipid used in the instant disclosure is 1,2-Dioleoyl-sn-glycero-3-phosphoethanolamine (DOPE), 1,2-dioleoyl-sn-glycero-3-phosphocholine (DOPC), and/or 1,2-Distearoyl-sn-glycero-3-phosphocholine (DSPC). In some aspects, one or more non-cationic lipid of the instant particles is cholesterol (CHE), β-sitosterol, and/or derivatives thereof.

In some embodiments that employ PEG-conjugated lipids, the PEG-conjugated lipid is one or more of a polyethyleneglycol (PEG)-lipid conjugate, a polyamide (ATTA)-lipid conjugate, and a mixture thereof. In one aspect, the PEG-lipid conjugate is one or more of a PEG-dialkyloxypropyl (DAA), a PEG-diacylglycerol (DAG), a PEG-phospholipid, a PEG-ceramide, and a mixture thereof. In one aspect, the PEG-DAG conjugate is one or more of a PEG-dilauroylglycerol (C12), a PEG-dimyristoylglycerol (C14), a PEG-dipalmitoylglycerol (C16), and a PEG-distearoylglycerol (C18). In one aspect, the PEG-DAA conjugate is one or more of a PEG-dilauryloxypropyl (C12), a PEG-dimyristyloxypropyl (C14), a PEG-dipalmityloxypropyl (C16), and a PEG-distearyloxypropyl (C18). In some embodiments, PEG is 2-dimyristoyl-rac-glycero-3-methoxypolyethylene glycol-2000 (PEG-DMG) and/or 1,2-distearoyl-rac-glycero-3-methoxypolyethylene glycol-2000 (PEG-DSG).

In some embodiments, amphipathic lipids are included in particles of the instant disclosure. Amphipathic lipids may refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols.

A variety of methods for preparing lipid nanoparticles are known in the art, including e.g., those described in Szoka, et al., Ann. Rev. Biophys. Bioeng., 9:467 (1980); U.S. Pat. Nos. 4,186,183, 4,217,344, 4,235,871, 4,261,975, 4,485,054, 4,501,728, 4,774,085, 4,837,028, 4,946,787; PCT Publication No. WO 91/17424; Deamer and Bangham, Biochim. Biophys. Acta, 443:629-634 (1976); Fraley, et al., Proc. Natl. Acad. Sci. USA, 76:3348-3352 (1979); Hope, et al., Biochim. Biophys. Acta, 812:55-65 (1985); Mayer, et al., Biochim. Biophys. Acta, 858:161-168 (1986); Williams, et al., Proc. Natl. Acad. Sci., 85:242-246 (1988); Lipid nanoparticles, Marc J. Ostro, ed., Marcel Dekker, Inc., New York, 1983, Chapter 1; Hope, et al., Chem. Phys. Lip., 40:89 (1986); and Lipid nanoparticles: A Practical Approach, Torchilin, V. P. et al., ed., Oxford University Press (2003), and references cited therein. Suitable methods include, but are not limited to, sonication, extrusion, high pressure/homogenization, microfluidization, detergent dialysis, calcium-induced fusion of small lipid nanoparticle vesicles, and ether-infusion methods, all of which are well known in the art.

Lipid particles prepared according to methods as disclosed herein and as known in the art can in certain embodiments be stored for substantial periods of time prior to drug loading and administration to a patient. For example, lipid nanoparticles can be dehydrated, stored, and subsequently rehydrated and loaded with one or more active agents, prior to administration. Lipid nanoparticles may also be dehydrated after being loaded with one or more active agents. Dehydration can be accomplished by a variety of methods available in the art, including the dehydration and lyophilization procedures described, e.g., in U.S. Pat. Nos. 4,880,635, 5,578,320, 5,837,279, 5,922,350, 4,857,319, 5,376,380, 5,817,334, 6,355,267, and 6,475,517. In one embodiment, lipid nanoparticles are dehydrated using standard freeze-drying apparatus, i.e., they are dehydrated under low pressure conditions. Also, the lipid nanoparticles can be frozen, e.g., in liquid nitrogen, prior to dehydration. Sugars can be added to the LNP environment, e.g., to the buffer containing the lipid nanoparticles, prior to dehydration, thereby promoting the integrity of the lipid nanoparticle during dehydration. See, e.g., U.S. Pat. No. 5,077,056 or 5,736,155.

Lipid nanoparticles may be sterilized by conventional methods at any point during their preparation, including, e.g., after sizing or after generating a pH gradient.

### Therapeutic Agents

As disclosed herein, therapeutic agents may include any molecule or compound capable of exerting a desired effect on a cell, tissue, tumor, organ, or subject. Therapeutic agents may be any type of molecule or compound including, but not limited to, nucleic acids, peptides, polypeptides, small molecules, and mixtures thereof.

In some embodiments, the therapeutic agent may be a salt or derivative thereof. Therapeutic agents may be therapeutically active themselves, or they may be prodrugs, which become active upon further modification/alteration.

In some embodiments, the lipid-based compositions described herein may be associated with a nucleic acid such as, for example, an siRNA, Dicer-substrate dsRNA, shRNA, aiRNA, miRNA, antisense oligonucleotides, ribozymes, and immunostimulatory oligonucleotides.

Nucleic acid therapy has well-known, tremendous potential to treat diseases at the gene level. However, safe and effective delivery systems are essential for nucleic acid therapeutics. Non-specific delivery to organs and tissues often results in off-site effects and toxicity. Delivery of therapeutics to a specific organ of interest is a well-recognized need in the development of lipid-nanoparticles, as well as in drug development generally. The concept of only targeting the cause of a disease without harming other parts of the body was described by Ehrlich 120 years ago. However, extant methods do not provide defined or well-known methodologies for developing nanoparticles targeting specific tissues without introducing additional ligand-based targeting strategies. Organ-specific targeting of lipid nanoparticles based on the structural affinity of the lipid to the tissue, as now disclosed herein, therefore meets a well-established need in terms of reducing off-site effects and toxicity.

Nucleic acids associated with or encapsulated by LNPs may contain modifications including but not limited to those selected from the following group: 2′-O-methyl modified nucleotides, a nucleotide comprising a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide; internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2.′

In certain embodiments, the active agent is a mRNA or a vector capable expressing a mRNA in a cell.

In embodiments, the active agent is a CRISPR/Cas system. Optionally, a LNP of the instant disclosure can be formulated to include, e.g., both a guide strand (gRNA) and a Cas enzyme as cargoes, thereby providing a self-contained delivery vehicle capable of effecting and controlling CRISPR-mediated targeting of a gene in a target cell.

In certain featured embodiments, the active agent is a nucleic acid modulating controller (e.g., a mRNA that encodes protein controller components, as described above).

In some embodiments, the active agent is a therapeutic agent, or a salt or derivative thereof. Therapeutic agent derivatives may be therapeutically active themselves or they may be prodrugs, which become active upon further modification. Thus, in one embodiment, a therapeutic agent derivative retains some or all of the therapeutic activity as compared to the unmodified agent, while in another embodiment, a therapeutic agent derivative lacks therapeutic activity.

In various embodiments, therapeutic agents include agents and drugs, such as anti-inflammatory compounds, narcotics, depressants, anti-depressants, stimulants, hallucinogens, analgesics, antibiotics, birth control medication, antipyretics, vasodilators, anti-angiogenics, cytovascular agents, signal transduction inhibitors, vasoconstrictors, hormones, and steroids.

In certain embodiments, the active agent is an oncology drug, which may also be referred to as an anti-tumor drug, an anti-cancer drug, a tumor drug, an antineoplastic agent, or the like. Examples of oncology drugs that may be used according to the instant disclosure include, but are not limited to, adriamycin, alkeran, allopurinol, altretamine, amifostine, anastrozole, araC, arsenic trioxide, azathioprine, bexarotene, biCNU, bleomycin, busulfan intravenous, busulfan oral, capecitabine (Xeloda), carboplatin, carmustine, CCNU, celecoxib, chlorambucil, cisplatin, cladribine, cyclosporin A, cytarabine, cytosine arabinoside, daunorubicin, cytoxan, daunorubicin, dexamethasone, dexrazoxane, dodetaxel, doxorubicin, doxorubicin, DTIC, epirubicin, estramustine, etoposide phosphate, etoposide and VP-16, exemestane, FK506, fludarabine, fluorouracil, 5-FU, gemcitabine (Gemzar), gemtuzumab-ozogamicin, goserelin acetate, hydrea, hydroxyurea, idarubicin, ifosfamide, imatinib mesylate, interferon, irinotecan (Camptostar, CPT-111), letrozole, leucovorin, leustatin, leuprolide, levamisole, litretinoin, megastrol, melphalan, L-PAM, mesna, methotrexate, methoxsalen, mithramycin, mitomycin, mitoxantrone, nitrogen mustard, paclitaxel, pamidronate, Pegademase, pentostatin, porfimer sodium, prednisone, rituxan, streptozocin, STI-571, tamoxifen, taxotere, temozolamide, teniposide, VM-26, topotecan (Hycamtin), toremifene, tretinoin, ATRA, valrubicin, velban, vinblastine, vincristine, VP16, and vinorelbine. Other examples of oncology drugs that may be used according to the instant disclosure are ellipticin and ellipticin analogs or derivatives, epothilones, intracellular kinase inhibitors and camptothecins.

While LNP compositions of the instant disclosure generally comprise a single active agent, in certain embodiments, they may comprise more than one active agent.

In other embodiments of the instant disclosure, the lipid nanoparticles of the instant disclosure have a plasma circulation half-life of at least 0.5, 0.8, 1.2, 1.5, 2.0, 4.0, 6.0, 8.0, or 12 hours. In some embodiments, lipid nanoparticles have a plasma drug half-life of at least 0.5, 0.8, 1.2, 1.5, 2.0, 4.0, 6.0, 8.0, or 12 hours. Circulation and blood or plasma clearance half-lives may be determined as described, for example, in U.S. Patent Publication No. 2004-0071768-A1.

The techniques herein further comprise lipid particles and/or pharmaceutical compositions in which a therapeutic agent such as, for example, nucleic acids (e.g., siRNA, ASO, tRNA, miRNA, mRNA, DNA, and the like), proteins, peptides, and other macromolecules, is enclosed within the lipid portion of the particle or composition so that it is protected from degradation. Such lipid particles and/or pharmaceutical compositions may be formed by any method known in the art including, but not limited to, a continuous mixing method, a direct dilution process, and an in-line dilution process.

In some embodiments, lipid particles and/or pharmaceutical compositions may include any of the reversible zwitterionic lipids disclosed herein, or salts thereof, alone or in combination with other cationic lipids and/or non-cationic lipids. In other embodiments, the non-cationic lipids may be egg sphingomyelin (ESM), distearoylphosphatidylcholine (DSPC), dioleoylphosphatidylcholine (DOPC), 1-palmitoyl-2-oleoyl-phosphatidylcholine (POPC), dipalmitoyl-phosphatidylcholine (DPPC), monomethyl-phosphatidylethanolamine, dimethyl-phosphatidylethanolamine, 14:0 PE (1,2-dimyristoyl-phosphatidylethanolamine (DMPE)), 16:0 PE (1,2-dipalmitoyl-phosphatidylethanolamine (DPPE)), 18:0 PE (1,2-distearoyl-phosphatidylethanolamine (DSPE)), 18:1 PE (1,2-dioleoylphosphatidylethanolamine (DOPE)), 18:1 trans PE (1,2-dielaidoyl-phosphatidylethanolamine (DEPE)), 18:0-18:1 PE (1-stearoyl-2-oleoyl-phosphatidylethanolamine (SOPE)), 16:0-18:1 PE (1-palmitoyl-2-oleoyl-phosphatidylethanolamine (POPE)), polyethylene glycol-based polymers (e.g., PEG 2000, PEG 5000, PEG-modified diacylglycerols, or PEG-modified dialkyloxypropyls), cholesterol, derivatives thereof, or combinations thereof.

The lipid particles and/or pharmaceutical compositions disclosed herein may be formed using techniques know in the art such as, for example, continuous mixing in which the process of continuously introducing lipid and buffer solutions into a mixing area causes a continuous dilution of the lipid solution with the buffer solution, which has the effect of producing a lipid vesicle almost immediately upon mixing. By mixing an aqueous solution comprising a therapeutic agent with an organic lipid solution, the organic lipid solution may undergo a continuous stepwise dilution in the presence of the buffer solution to produce a therapeutic agent-lipid particle. Such particles may have a size of from about 30 nm to about 250 nm, from about 40 nm to about 200 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, less than about 120 nm, 110 nm, 100 nm, 90 nm, or 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, or 250 nm, or any intermediate value or sub-range therein. Once formed, the particles do not aggregate. According to the techniques herein, the particles may be sized to achieve a uniform particle size.

It is also contemplated within the scope of the disclosure that such particles may be prepared by a direct dilution process (e.g., forming a lipid vesicle solution and directly introducing it into a container having a controlled amount of dilution buffer) such as is described in U.S. Patent Publication No. 20070042031, the disclosure of which is herein incorporated by reference in its entirety for all purposes. The particles formed using the direct dilution processes typically have a size of from about 30 nm to about 250 nm, from about 40 nm to about 200 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, less than about 120 nm, 110 nm, 100 nm, 90 nm, or 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, or 250 nm, or any intermediate value or sub-range therein. Once formed, the particles do not aggregate. According to the techniques herein, the particles may be sized to achieve a uniform particle size.

In some embodiments, non-lipid polycations which are useful to effect the lipofection of cells may be added to the present compositions. Examples of suitable non-lipid polycations include, hexadimethrine bromide (sold under the brand name POLYBRENE®, from Aldrich Chemical Co., Milwaukee, Wis., USA) or other salts of hexadimethrine. Other suitable polycations include, for example, salts of poly-L-ornithine, poly-L-arginine, poly-L-lysine, poly-D-lysine, polyallylamine, and polyethyleneimine. Addition of these salts is preferably after the particles have been formed.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Synthesis of OMGT-014, Aka SM-007

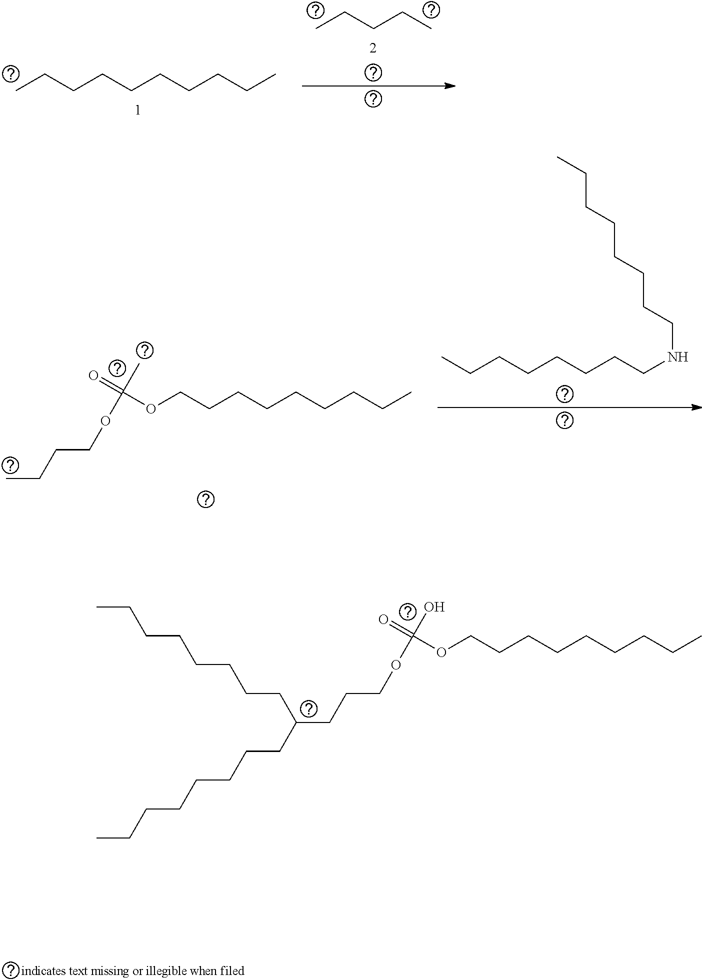

### Step 1: 3-bromopropyl Nonyl Hydrogen Phosphate (3): (EC5059-55/59)

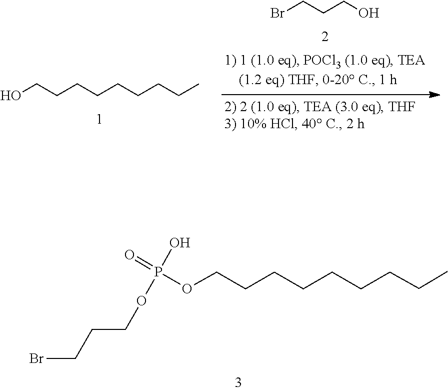

To a solution of TEA (6.31 g, 62.39 mmol, 8.7 mL, 1.2 eq) dry THF (150 mL) was added to POCl₃ (7.97 g, 51.99 mmol, 4.8 mL, 1 eq) slowly at 0° C. under N₂. Then nonan-1-ol (7.5 g, 51.99 mmol, 1 eq) in THF (100 mL) was added dropwise over 1 hour and the resulting mixture was warmed to 20° C. and was stirred for 1 hour. When all of the alcohol was completely consumed (as checked by TLC), the mixture was cooled to 0° C. and a second portion of TEA (15.78 g, 155.98 mmol, 21.7 mL, 3 eq) was added, followed by 3-bromopropan-1-ol (7.23 g, 51.99 mmol, 4.7 mL, 1 eq) in THF (50 mL) that was added dropwise. The reaction mixture was stirred at 20° C. for 14 hours. The mixture was decomposed with HCl (10%, 100 mL) and heated at 40° C. for 2 hours. THF was removed under vacuum and the aqueous residue was extracted with DCM (150 mL×3). The organic layer was dried over Na₂SO₄, filtered, and reduced under vacuum. The residue was purified by flash silica gel chromatography (120 g SepaFlash® Silica Flash Column, (DCM:MeOH: 0˜8%) to give compound 3-bromopropyl nonyl hydrogen phosphate (4.90 g, 14.19 mmol, 49.0% yield) as a yellow oil.

¹H NMR (400 MHz, CDCl3) δ=9.62 (s, 1H), 4.25-4.15 (m, 2H), 4.08-4.01 (m, 2H), 3.57-3.49 (m, 2H), 2.25-2.18 (m, 2H), 1.74-1.66 (m, 2H), 1.41-1.26 (m, 12H), 0.89 (t, J=6.0 Hz, 3H).

### Step 2: 3-(dioctylamino)propyl Nonyl Hydrogen Phosphate (SM-007): (EC5059-61/65)

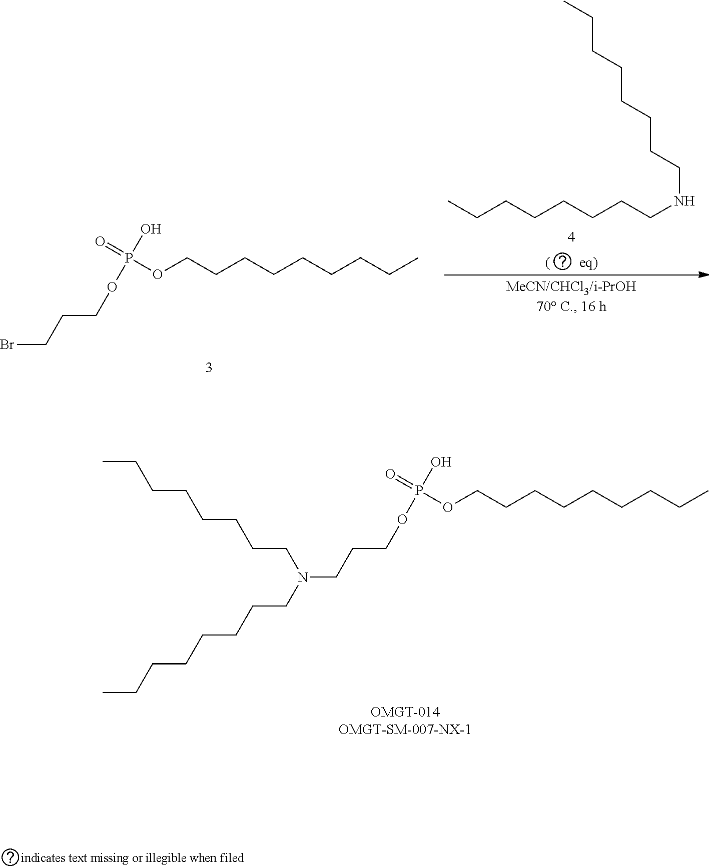 [SMILES: CCCCCCCCNCCCCCCCC ; CCCCCCCCCOP(=O)(O)OCCCBr ; CCCCCCCCCOP(=O)(O)OCCCN(CCCCCCCC)CCCCCCCC]

A mixture of N-octyloctan-1-amine (12.59 g, 52.14 mmol, 6 eq) and 3-bromopropyl nonyl hydrogen phosphate (3 g, 8.69 mmol, 1 eq) in MeCN (2 mL), CHCl₃ (2 mL) and i-PrOH (2 mL) were stirred at 70° C. for 16 hours. The reaction mixture was directly concentrated under reduced pressure to give a residue. The residue was diluted with DCM (150 mL) and washed with HCl (10%, 50 mL×2), dried over Na₂SO₄, filtered and concentrated under reduced pressure to give a residue. The residue was purified by flash silica gel chromatography (80 g SepaFlash® Silica Flash Column, (DCM:MeOH: 0˜10%) to give compound 3-(dioctylamino)propyl nonyl hydrogen phosphate (3.2 g, crude) as a yellow oil, and then the crude product was purified by flash silica gel chromatography (40 g SepaFlash® Silica Flash Column, DCM:MeOH: 0˜5%) to give pure compound 3-(dioctylamino)propyl nonyl hydrogen phosphate aka SM-007 (680.00 mg, 1.33 mmol, 45.0% yield, 99.2% purity) as a white solid.

¹H NMR (400 MHz, CDCl3) δ=13.39 (s, 1H), 4.05-3.96 (m, 2H), 3.92-3.84 (m, 2H), 3.13-3.06 (m, 2H), 2.96-2.84 (m, 4H), 2.07-1.95 (m, 2H), 1.76-1.58 (m, 6H), 1.36-1.22 (m, 32H), 0.92-0.82 (m, 9H).

### Example 2: Synthesis of OMGT-015, Aka SM-008

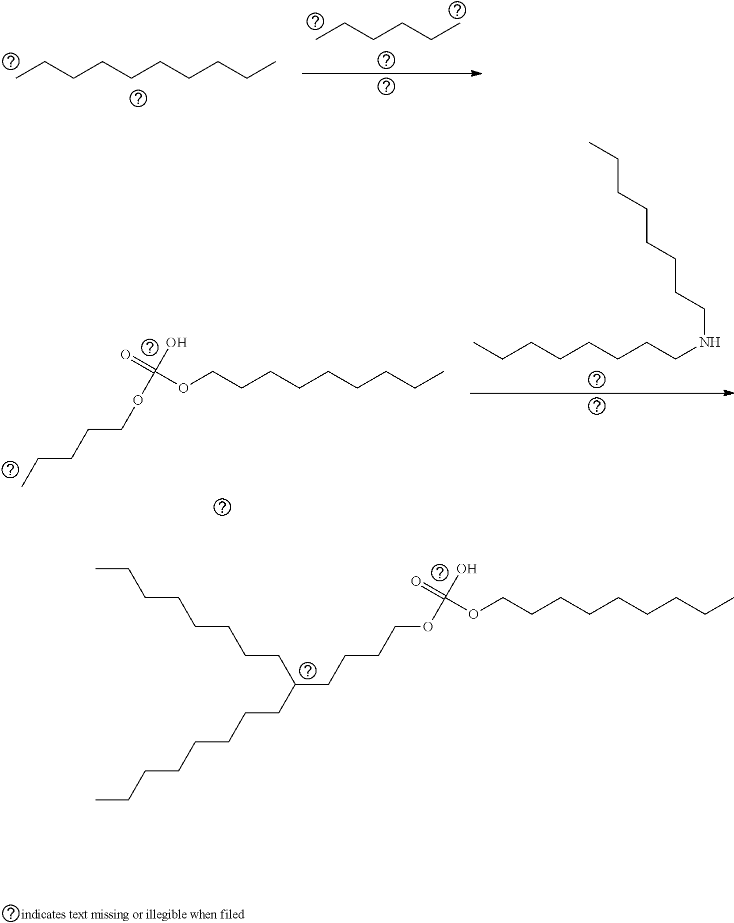

### Step 1: 4-bromobutyl Nonyl Hydrogen Phosphate: (EC5500-29)

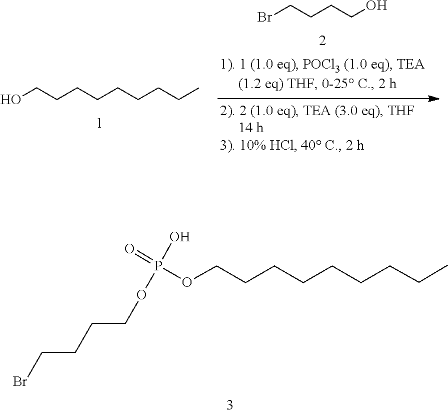

To a solution of TEA (8.42 g, 83.19 mmol, 11.58 mL, 1.2 eq) in dry THF (200 mL) was slowly added POCl₃ (10.63 g, 69.32 mmol, 6.44 mL, 1 eq) at 0° C. under N₂. Then nonan-1-ol (10 g, 69.32 mmol, 1 eq) in THF (150 mL) was added to the above reaction mixture dropwise over 1 hour, and the resulting mixture was heated to 20° C. with stirring for 1 hour. Then the mixture was cooled to 0° C. and a second portion of TEA (21.04 g, 207.97 mmol, 28.95 mL, 3 eq) was added, followed by 4-bromobutan-1-ol (10.61 g, 69.32 mmol, 1 eq) in THF (50 mL) which was added dropwise. The reaction mixture was stirred at 20° C. for 14 hour. The reaction mixture was decomposed with 10% HCl (100 mL) and stirred at 40° C. for 2 hours. THF was removed under vacuum and the aqueous residue was extracted with DCM (150 mL×3). The organic layer was dried over Na₂SO₄, filtered, concentrated under vacuum. The residue was purified by flash silica gel chromatography (ISCO®; 80 g SepaFlash® Silica Flash Column, Eluent of 0˜10% DCM/MeOH gradient @80 mL/min). Compound 4-bromobutyl nonyl hydrogen phosphate (6.70 g, 14.92 mmol, 21.52% yield, 80% purity) was obtained as a white solid.

¹H NMR (400 MHz, CDCl3) δ=4.45-3.73 (m, 4H), 3.58-3.30 (m, 2H), 2.22-1.59 (m, 6H), 1.56-1.06 (m, 12H), 0.89 (t, J=7.2 Hz, 3H).

### Step 2: 4-(dioctylamino)butyl Nonyl Hydrogen Phosphate: (EC5500-33/34)

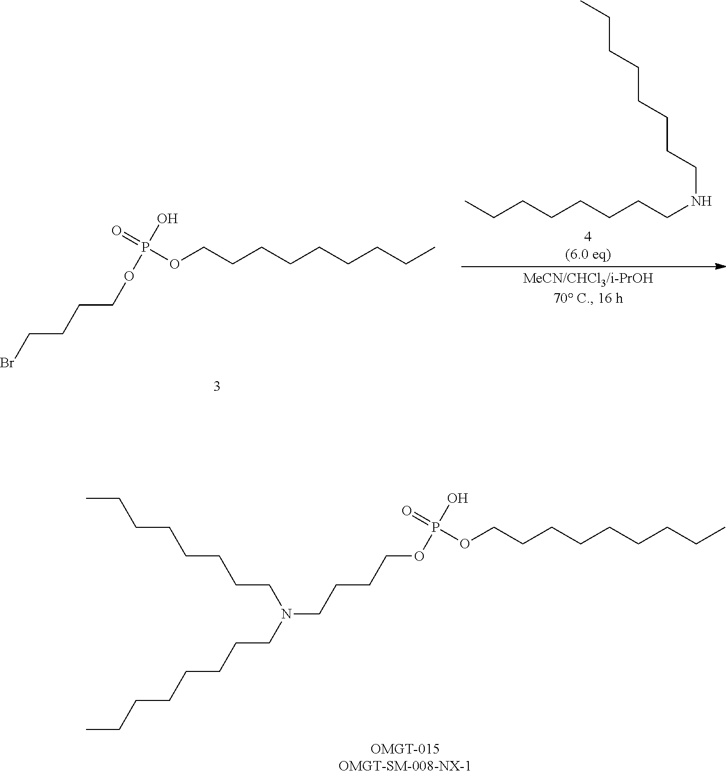 [SMILES: CCCCCCCCNCCCCCCCC ; CCCCCCCCCOP(=O)(O)OCCCCBr ; CCCCCCCCCOP(=O)(O)OCCCCN(CCCCCCCC)CCCCCCCC]

To a solution of 4-bromobutyl nonyl hydrogen phosphate (2.4 g, 6.68 mmol, 1 eq) was added N-octyloctan-1-amine (9.68 g, 40.09 mmol, 6 eq) in MeCN (2 mL), CHCl₃ (2 mL) and i-PrOH (2 mL). The mixture was stirred at 70° C. for 16 hours. The reaction mixture was directly concentrated under reduced pressure to give a residue. The residue was diluted with DCM (150 mL) and washed with 10% HCl solution (50 mL×2), dried over Na₂SO₄, filtered and concentrated under reduced pressure to give a residue. The residue was purified by flash silica gel chromatography (80 g SepaFlash® Silica Flash Column, (Dichloromethane:Methanol: 0˜10%) to yield compound 4-(dioctylamino)butyl nonyl hydrogen phosphate aka SM-008 (1.20 g, 2.31 mmol, 34.56% yield) was obtained as a colorless oil.

LCMS: [M+H]+: 520.6

¹H NMR (400 MHz, CDCl3) δ=13.51-12.29 (brs, 1H), 4.02-3.70 (m, 4H), 3.09-2.84 (m, 6H), 1.98-1.86 (m, 2H), 1.76-1.54 (m, 8H), 1.39-1.21 (m, 32H), 1.01-0.76 (m, 9H).

### Example 3: Synthesis of OMGT-031, Aka SM-009

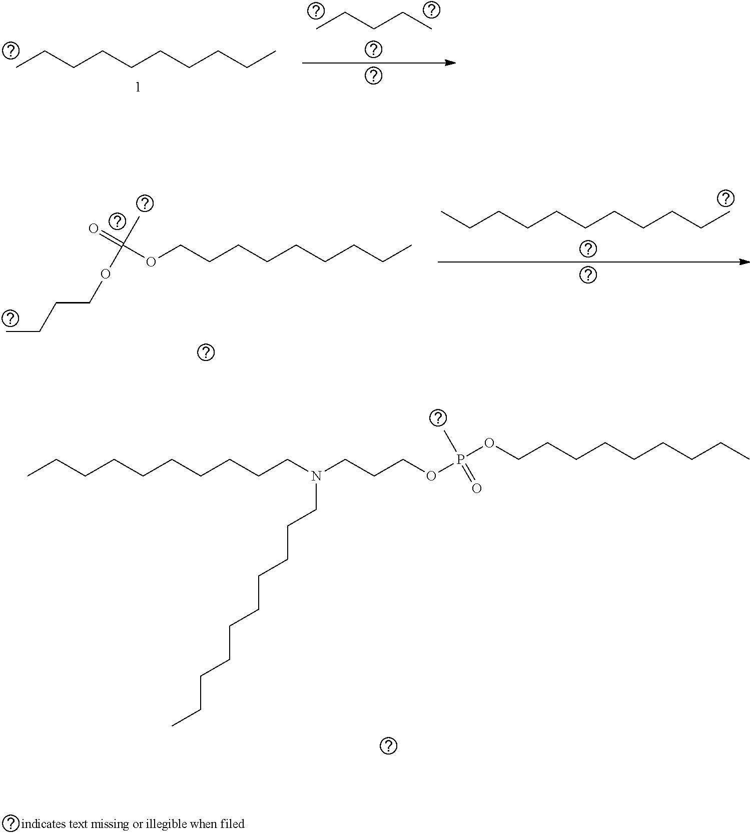

### Step 1: 3-bromopropyl Nonyl Hydrogen Phosphate: (EC5000-66/EC5000-89)

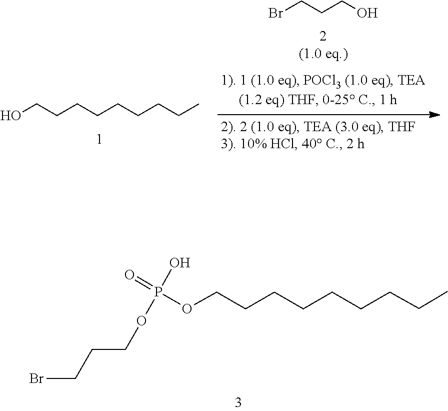

To a solution of TEA (42.09 g, 415.93 mmol, 57.89 mL, 3 eq) in dry THF (150 mL) was slowly added POCl₃ (21.26 g, 138.64 mmol, 12.88 mL, 1 eq) at 0° C. under N₂. Then nonan-1-ol (20.00 g, 138.64 mmol, 1 eq) in THF (150 mL) was added dropwise over 1 hour, the resulting mixture was warmed to 20° C. and stirred for 1 hour. When all of the alcohol had reacted (as checked by TLC), the mixture was cooled to 0° C. and a second portion of TEA (42.09 g, 415.93 mmol, 57.89 mL, 3 eq) was added, followed by 3-bromopropan-1-ol (19.27 g, 138.64 mmol, 12.51 mL, 1 eq) in THF (150 mL), which was added dropwise. The reaction mixture was stirred at 20° C. for 14 hours, decomposed with HCl (10%, 200 mL) and heated at 40° C. for 2 hours. THF was removed under vacuum and the aqueous residue was extracted with EA (300 mL×3). The organic layer was dried over Na₂SO₄, filtered, reduced under vacuum. The residue was purified by column chromatography (SiO₂, DCM/MeOH=1/0 to 5/1) to give compound 3-bromopropyl nonyl hydrogen phosphate (8.70 g, 25.20 mmol, 29.00% yield) as a yellow oil.

¹H NMR (400 MHz, CDCl3) δ=4.42-3.79 (m, 4H), 3.68-3.35 (m, 2H), 2.48-2.04 (m, 2H), 1.72-1.53 (m, 2H), 1.45-1.15 (m, 12H), 0.89 (t, J=6.4 Hz, 3H).

### Step 2: 3-(didecylamino)propyl Nonyl Hydrogen Phosphate: (EC5000-91/EC5000-99)

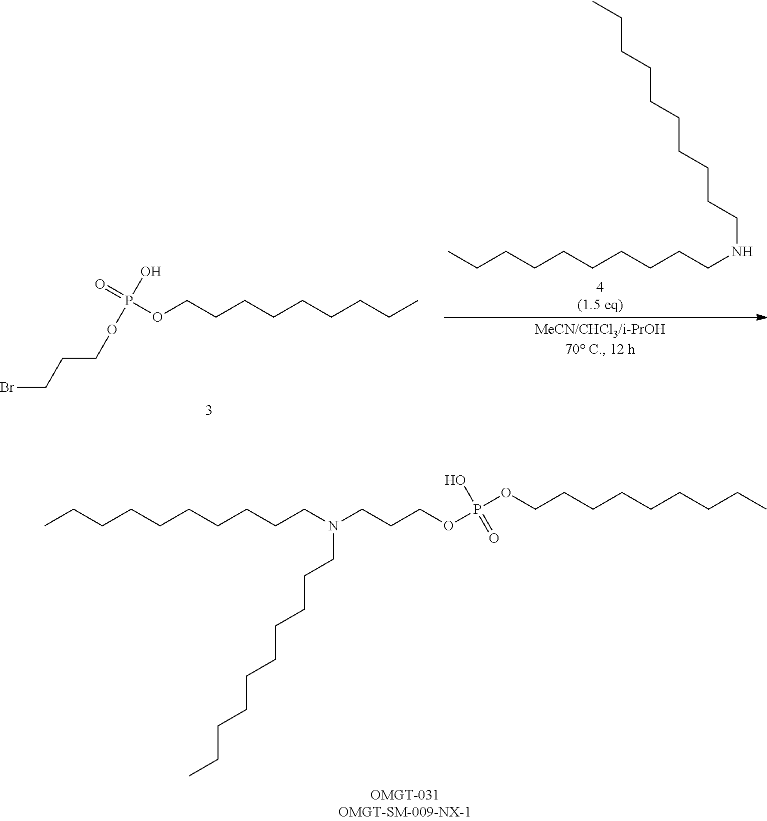 [SMILES: CCCCCCCCCCNCCCCCCCCCC ; CCCCCCCCCOP(=O)(O)OCCCBr ; CCCCCCCCCCN(CCCCCCCCCC)CCCOP(=O)(O)OCCCCCCCCC]

To a solution of 3-bromopropyl nonyl hydrogen phosphate (2.00 g, 5.79 mmol, 1 eq) in MeCN (2 mL), CHCl₃ (2 mL) and i-PrOH (2 mL) was added N-decyldecan-1-amine (2.59 g, 8.69 mmol, 1.5 eq). The mixture was stirred at 70° C. for 12 hours. The reaction mixture was concentrated under reduced pressure to give a residue. The residue was diluted with MeCN (70 mL) and washed with HCl solution (10%, 50 mL×2), dried over Na₂SO₄, filtered and concentrated under reduced pressure to give a residue. The residue was purified by column chromatography (SiO₂, DCM/MeOH=30/1 to 10/1) to give compound 3-(didecylamino)propyl nonyl hydrogen phosphate aka SM-009 (220 mg, 391.56 umol, 56.32% yield) as a yellow oil.

¹H NMR (400 MHz, CDCl3) δ=13.80-12.62 (brs, 1H), 4.19-4.06 (m, 2H), 3.98-3.83 (m, 2H), 3.21-3.11 (m, 2H), 2.98-2.79 (m, 4H), 2.11-1.97 (m, 2H), 1.82-1.52 (m, 6H), 1.49-1.09 (m, 40H), 0.87 (t, J=6.4 Hz, 9H).

### Example 4: Synthesis of OMGT-032, Aka SM-010

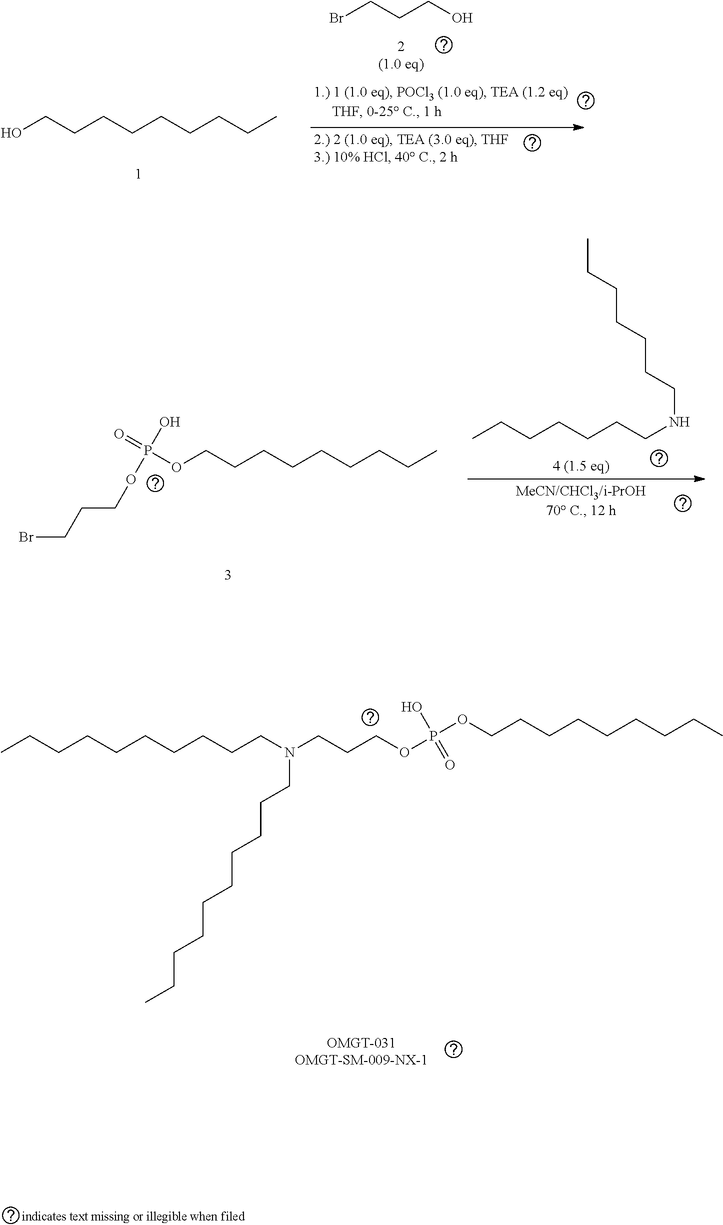

### Step 1: 3-bromopropyl Nonyl Hydrogen Phosphate: (EC5000-66/EC5000-89)

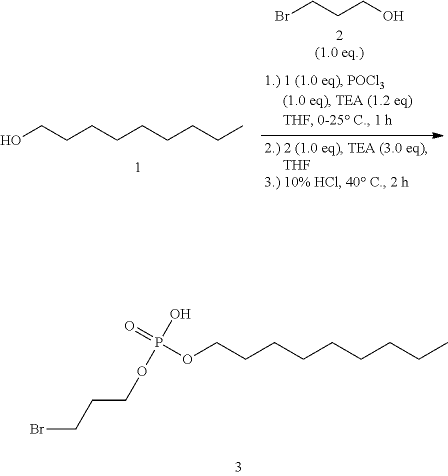

To a solution of TEA (42.09 g, 415.93 mmol, 57.89 mL, 3 eq) in dry THF (150 mL) was slowly added POCl₃ (21.26 g, 138.64 mmol, 12.88 mL, 1 eq) at 0° C. under N₂. Then nonan-1-ol (20 g, 138.64 mmol, 1 eq) in THF (150 mL) was added dropwise over 1 hour, and the resulting mixture was warmed to 20° C. and stirred for 1 hour. When all the alcohol had reacted (as checked by TLC), the mixture was cooled to 0° C. and a second portion of TEA (42.09 g, 415.93 mmol, 57.89 mL, 3 eq) was added, followed by 3-bromopropan-1-ol (19.27 g, 138.64 mmol, 12.51 mL, 1 eq) in THF (150 mL), which was added dropwise. The reaction mixture was stirred at 20° C. for 14 hours. Decomposed with HCl (10%, 200 mL) and heated at 40° C. for 2 hours. THF was removed under vacuum and the aqueous residue was extracted with EA (300 mL×3). The organic layer was dried over Na₂SO₄, filtered, reduced under vacuum. The residue was purified by column chromatography (SiO₂, DCM/MeOH=1/0 to 5/1) to give compound 3-bromopropyl nonyl hydrogen phosphate (8.70 g, 25.20 mmol, 29.00% yield) as a yellow oil.

¹H NMR (400 MHz, CDCl3) δ=4.42-3.79 (m, 4H), 3.68-3.35 (m, 2H), 2.48-2.04 (m, 2H), 1.72-1.53 (m, 2H), 1.45-1.15 (m, 12H), 0.89 (t, J=6.4 Hz, 3H).

### Step 2: 3-(diheptylamino)propyl Nonyl Hydrogen Phosphate: (EC5000-93/EC5000-108)

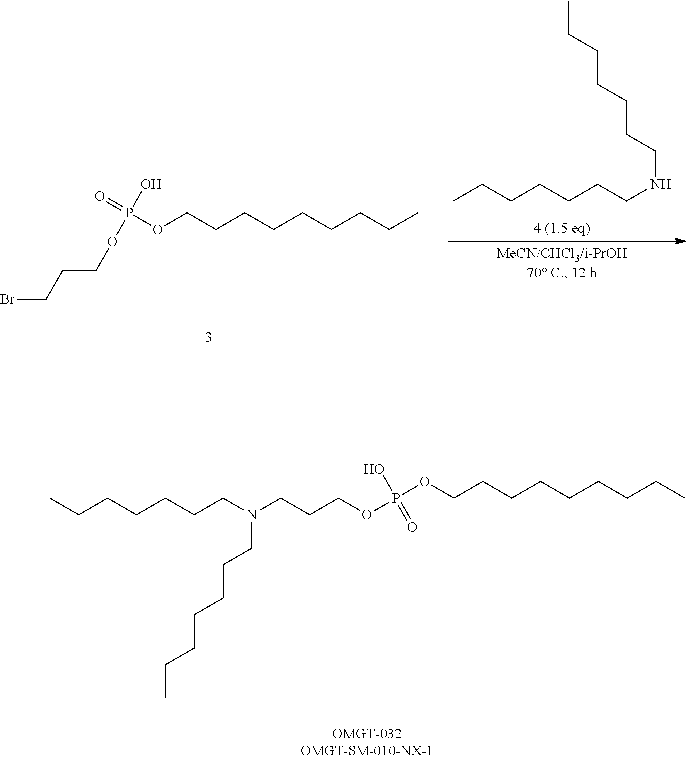

To a solution of 3-bromopropyl nonyl hydrogen phosphate (2.60 g, 7.53 mmol, 1 eq) in MeCN (1.5 mL), CHCl₃ (1.5 mL) and i-PrOH (1.5 mL) was added N-heptylheptan-1-amine (2.41 g, 11.30 mmol, 1.5 eq). The mixture was stirred at 70° C. for 12 hours. The reaction mixture was directly concentrated under reduced pressure to give a residue. The residue was diluted with MeCN (150 mL) and washed with HCl solution (10%, 50 mL×2), dried over Na₂SO₄, filtered and concentrated under reduced pressure to give a residue. The residue was purified by column chromatography (SiO₂, DCM/MeOH=30/1 to 10/1) to give compound 3-(diheptylamino)propyl nonyl hydrogen phosphate aka SM-010 (500.00 mg, 1.03 mmol, 46.89% yield, 98% purity) as a yellow oil.

¹H NMR (400 MHz, CDCl3) δ=13.79-13.06 (brs, 1H), 4.19-4.06 (m, 2H), 3.98-3.83 (m, 2H), 3.21-3.11 (m, 2H), 2.98-2.79 (m, 4H), 2.09-1.97 (m, 2H), 1.82-1.52 (m, 6H), 1.49-1.19 (m, 28H), 0.88 (t, J=6.4 Hz, 9H).

### Example 5: Synthesis of OMGT-033, Aka SM-012

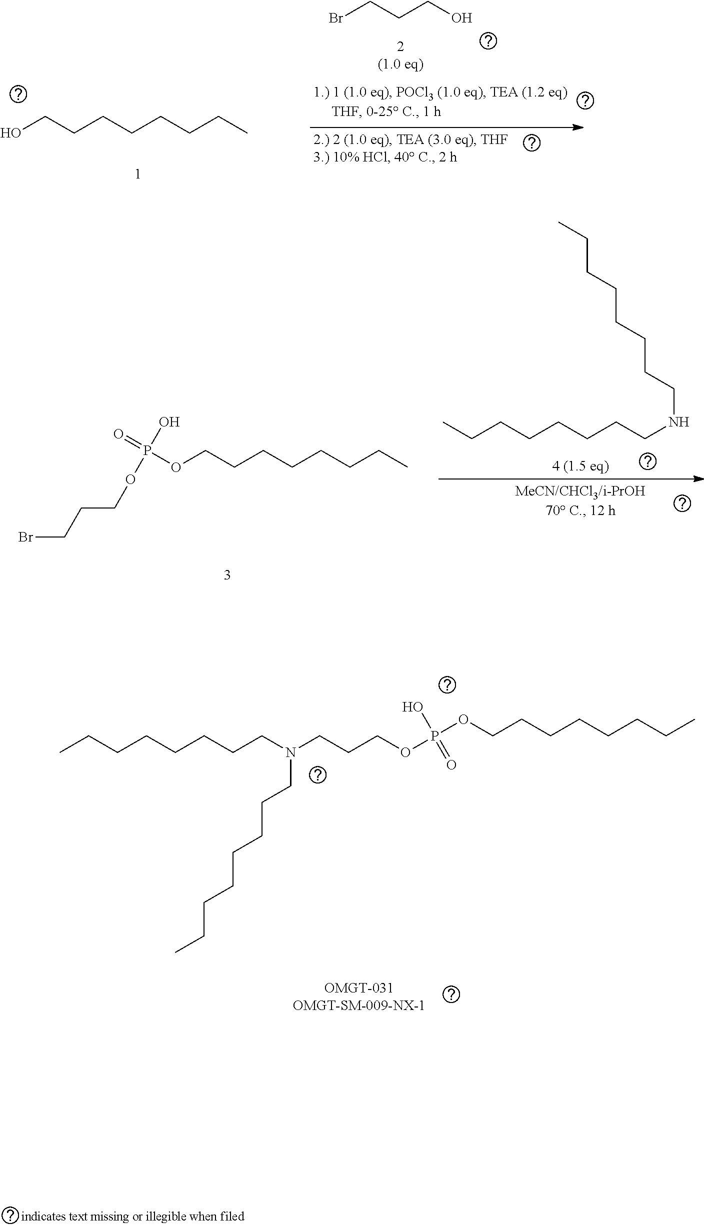

### Step 1: 3-bromopropyl Octyl Hydrogen Phosphate: (EC5500-45)

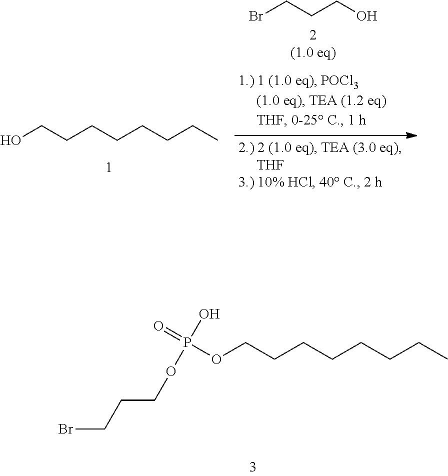

To a solution of POCl₃ (11.77 g, 76.79 mmol, 7.14 mL, 1 eq) in THF (200 mL) was slowly added TEA (9.32 g, 92.15 mmol, 12.83 mL, 1.2 eq) at 0° C. under N₂. Then the solution of octan-1-ol (10.00 g, 76.79 mmol, 12.14 mL, 1 eq) in THF (150 mL) was added dropwise over 1 hour, and the resulting mixture was warmed to 20° C. and stirred for 1 hour. When all the alcohol had reacted (as checked by TLC), the mixture was cooled to 0° C. and a second portion of TEA (23.31 g, 230.37 mmol, 32.06 mL, 3 eq) was added, followed by the solution of 3-bromopropan-1-ol (10.67 g, 76.79 mmol, 6.93 mL, 1 eq) in THF (50 mL), which was added dropwise. The reaction mixture was stirred at 20° C. for 14 hours. The reaction mixture was decomposed with HCl (10%, 100 mL) and heated at 40° C. for 2 hours. THF was removed under vacuum and the aqueous residue was extracted with DCM (150 mL×3). The organic layer was dried over Na₂SO₄, filtered, concentrated under reduced pressure. The residue was purified by flash silica gel chromatography (ISCO®; 80 g SepaFlash® Silica Flash Column, Eluent of 0˜10% DCM/MeOH gradient @80 mL/min) to give compound 3-bromopropyl octyl hydrogen phosphate (7.00 g, 21.14 mmol, 27.53% yield) as a white solid.

¹H NMR (400 MHz, CDCl3) δ=4.40-3.91 (m, 4H), 3.65-3.42 (m, 2H), 2.34-2.08 (m, 2H), 1.79-1.60 (m, 2H), 1.42-1.19 (m, 10H), 0.89 (t, J=6.4 Hz, 3H).

### Step 2: 3-(dioctylamino)propyl Octyl Hydrogen Phosphate: (EC5000-101/EC5000-106)

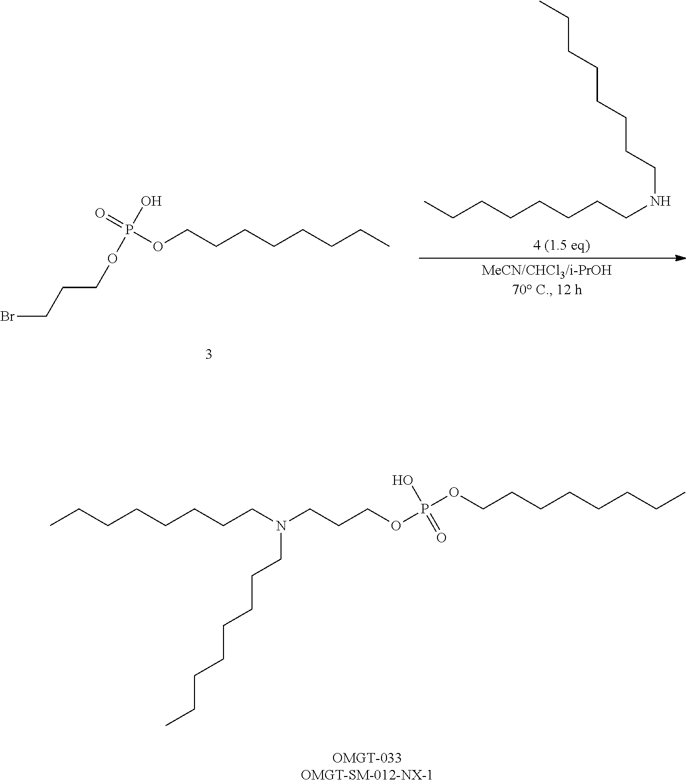 [SMILES: CCCCCCCCOP(=O)(O)OCCCBr ; CCCCCCCCNCCCCCCCC ; CCCCCCCCOP(=O)(O)OCCCN(CCCCCCCC)CCCCCCCC]

To a solution of 3-bromopropyl octyl hydrogen phosphate (3.00 g, 9.06 mmol, 1 eq) in MeCN (2 mL), CHCl₃ (2 mL) and i-PrOH (2 mL) was added N-octyloctan-1-amine (3.28 g, 13.59 mmol, 1.5 eq). The mixture was stirred at 70° C. for 12 hours. The reaction mixture was directly concentrated under reduced pressure to give a residue. The residue was diluted with DCM (100 mL) and washed with HCl solution (10%, 60 mL×2), dried over Na₂SO₄, filtered and concentrated under reduced pressure to give a residue. The residue was purified by column chromatography (SiO2, DCM/MeOH=30/1 to 10/1) to give compound 3-(dioctylamino)propyl octyl hydrogen phosphate aka SM-012 (350.00 mg, 711.78 umol, 36.84% yield, 100% purity) as a yellow oil.

LCMS: [M+H]+: 492.6

¹H NMR (400 MHz, CDCl3) δ=13.89-13.16 (brs, 1H), 4.21-4.06 (m, 2H), 3.98-3.81 (m, 2H), 3.31-3.02 (m, 2H), 2.98-2.71 (m, 4H), 2.09-1.97 (m, 2H), 1.79-1.56 (m, 6H), 1.42-1.19 (m, 30H), 0.89 (t, J=6.4 Hz, 9H).

### Example 6: Synthesis of OMGT-030, Aka SM-013

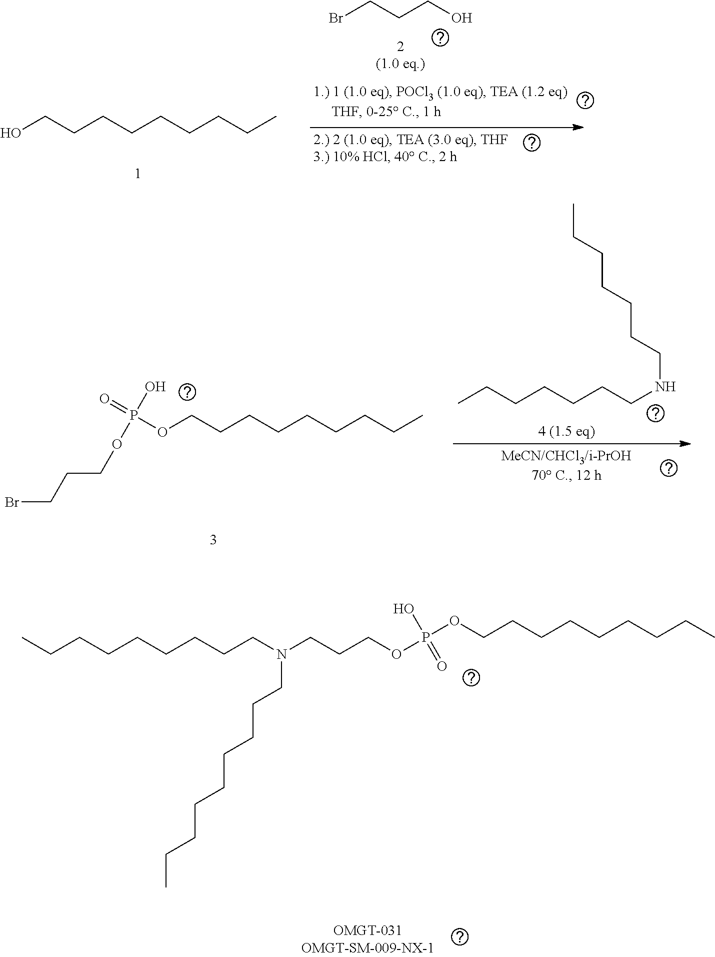

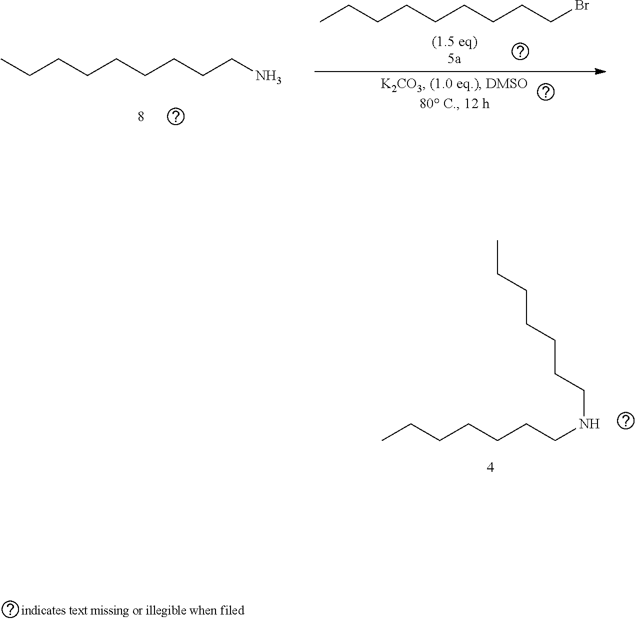

### Step 1: N-nonylnonan-1-amine: (EC5000-97)

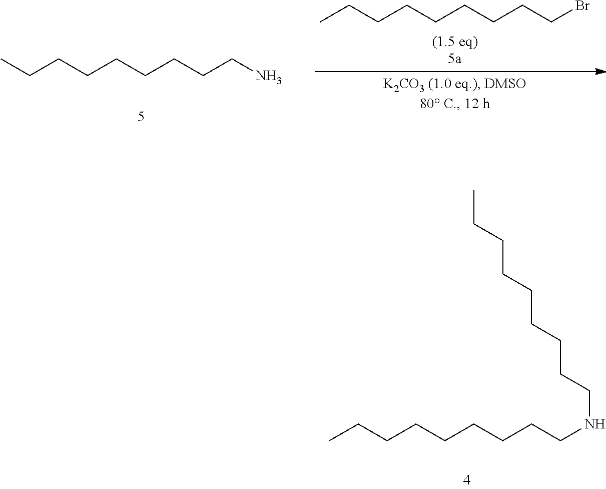

To a mixture of nonan-1-amine (9.00 g, 62.82 mmol, 1 eq) and 1-bromononane (26.03 g, 125.64 mmol, 2 eq) in DMSO (70 mL) was added K₂CO₃ (8.68 g, 62.82 mmol, 1 eq), the reaction mixture was stirred at 80° C. for 12 hours. TLC (PE/EtOAc/TEA=10:1:0.05) showed the reaction was completed. The reaction mixture was diluted with DCM (150 mL) and washed with water (3*100 mL). The organic layers were dried over Na₂SO₄, filtered and concentrated under reduced pressure to give a residue. The residue was purified by flash silica gel chromatography (220 g SepaFlash® Silica Flash Column, Petroleum ether:Ethyl acetate: 5˜30%, 1% TEA in EtOAc) to give compound N-nonylnonan-1-amine (8.10 g, 47.8% yield) as a yellow oil.

### Step 2: 3-bromopropyl Nonyl Hydrogen Phosphate: (EC5000-66/EC5000-89)

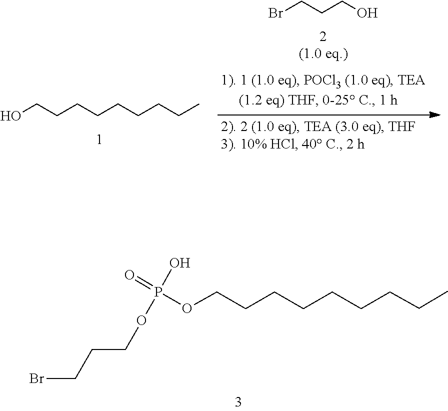

To a solution of TEA (42.09 g, 415.93 mmol, 57.89 mL, 3 eq) in dry THF (150 mL) was slowly added POCl₃ (21.26 g, 138.64 mmol, 12.88 mL, 1 eq) at 0° C. under N₂. Then nonan-1-ol (20.00 g, 138.64 mmol, 1 eq) in THF (150 mL) was added dropwise over 1 hour, the resulting mixture was warmed to 20° C. and stirred for 1 hour. When all the alcohol had reacted (as checked by TLC), the mixture was cooled to 0° C. and a second portion of TEA (42.09 g, 415.93 mmol, 57.89 mL, 3 eq) was added, followed by 3-bromopropan-1-ol (19.27 g, 138.64 mmol, 12.51 mL, 1 eq) in THF (150 mL), which was added dropwise. The reaction mixture was stirred at 20° C. for 14 hours. Decomposed with HCl (10%, 200 mL) and heated at 40° C. for 2 hours. THF was removed under vacuum and the aqueous residue was extracted with EA (300 mL×3). The organic layer was dried over Na₂SO₄, filtered, reduced under vacuum. The residue was purified by column chromatography (SiO₂, DCM/MeOH=1/0 to 5/1) to give compound 3-bromopropyl nonyl hydrogen phosphate (8.70 g, 25.20 mmol, 29.00% yield) as a yellow oil.

¹H NMR (400 MHz, CDCl3) δ=4.42-3.79 (m, 4H), 3.68-3.35 (m, 2H), 2.48-2.04 (m, 2H), 1.72-1.53 (m, 2H), 1.45-1.15 (m, 12H), 0.89 (t, J=6.4 Hz, 3H).

### Step 3: 3-[di(nonyl)amino]propyl Nonyl Hydrogen Phosphate: (EC5000-105/EC5000-112)

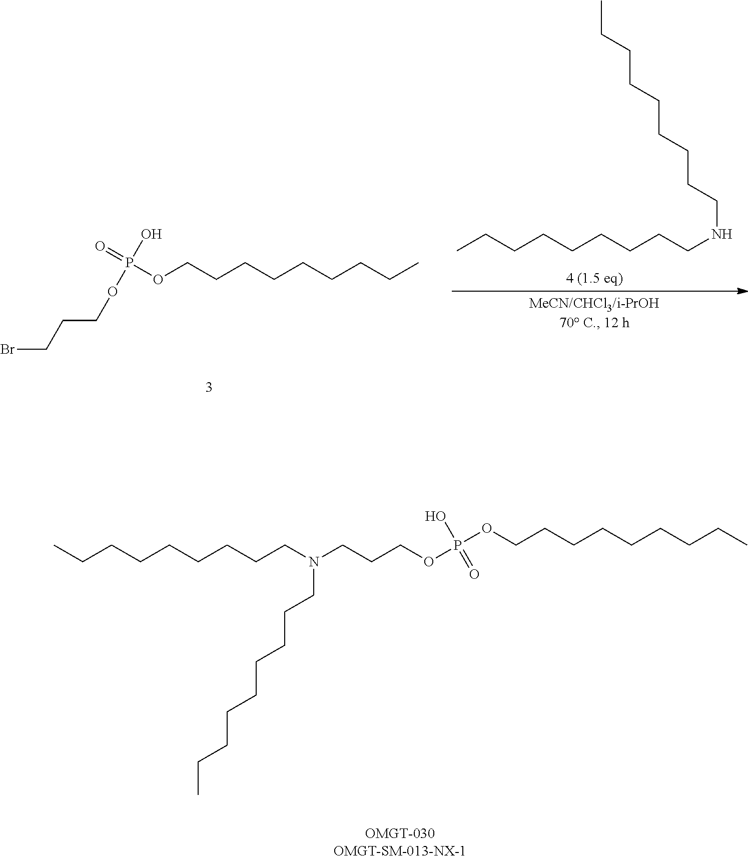

To a solution of 3-bromopropyl nonyl hydrogen phosphate (1.20 g, 3.48 mmol, 1 eq) in MeCN (1 mL), CHCl₃ (1 mL) and i-PrOH (1 mL) was added N-nonylnonan-1-amine (1.41 g, 5.21 mmol, 1.5 eq). The mixture was stirred at 70° C. for 12 hours. The reaction mixture was directly concentrated under reduced pressure to give a residue. The residue was diluted with DCM (60 mL) and washed with HCl solution (10%, 50 mL×2), dried over Na₂SO₄, filtered and concentrated under reduced pressure to give a residue. The residue was purified by column chromatography (SiO₂, DCM/MeOH=30/1 to 10/1) to give compound 3-(dinonylamino)propyl nonyl hydrogen phosphate aka SM-013 (240.00 mg, 444.70 umol, 24.73% yield, 98.91% purity) as a yellow oil.

¹H NMR (400 MHz, CDCl3) δ=13.89-13.06 (brs, 1H), 4.21-4.06 (m, 2H), 3.98-3.81 (m, 2H), 3.31-3.02 (m, 2H), 2.98-2.71 (m, 4H), 2.09-1.97 (m, 2H), 1.81-1.56 (m, 6H), 1.45-1.21 (m, 36H), 0.88 (t, J=6.4 Hz, 9H).

### Example 7: Synthesis of 4-(dioctylamino)but-2-yn-1-yl Nonyl Hydrogen Phosphate (OMGT-047, Aka SM-017)

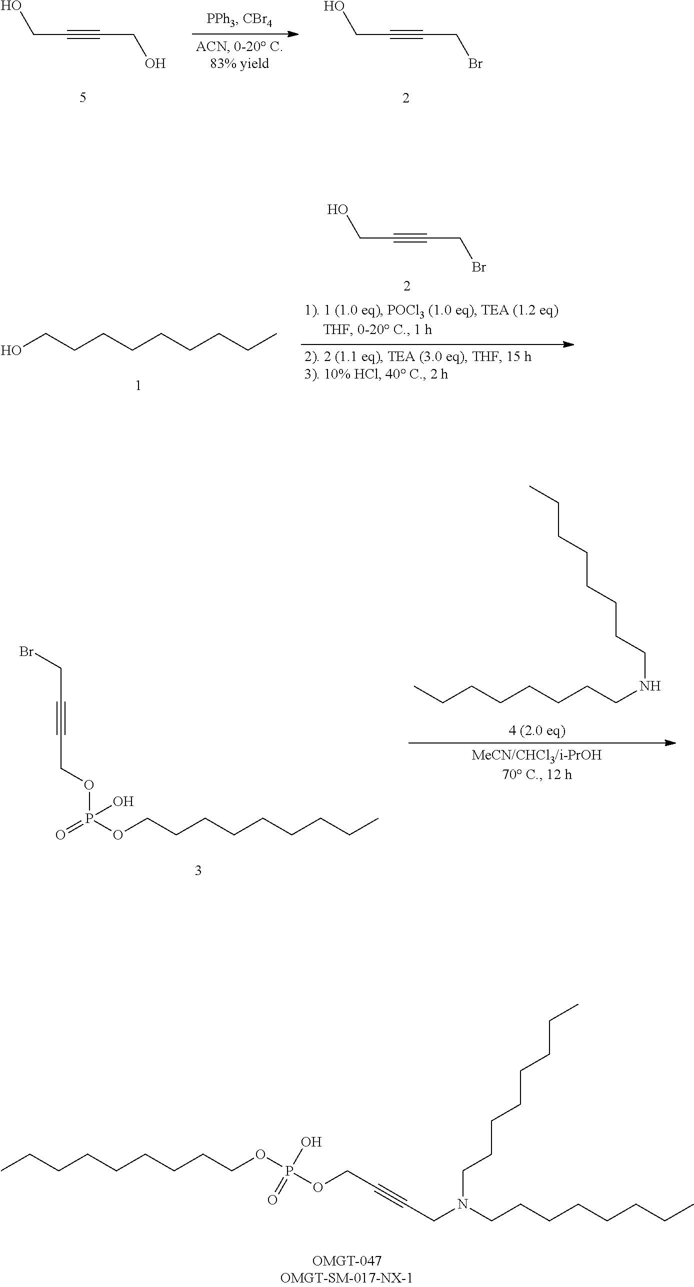

### Step 1: 4-bromobut-2-yn-1-ol (EC7119-4)

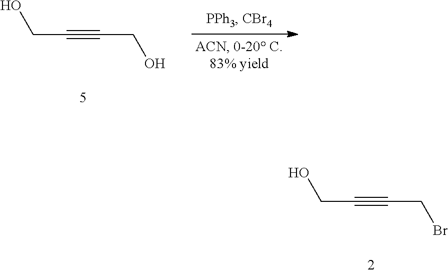 [SMILES: OCC#CCO ; OCC#CCBr]

To a solution of but-2-yne-1,4-diol (13.0 g, 151 mmol, 1.0 eq.) in ACN (150 mL) were added PPh₃ (59.4 g, 226 mmol, 1.5 eq.) and CBr₄ (60.1 g, 181 mmol, 1.2 eq.) at 0° C. Then the mixture was warmed to 20° C. and stirred for 1 h. After completion, the reaction mixture was diluted with H₂O (50 mL) and extracted with DCM (50 mL*2). The organic was washed with brine, dried with anhydrous sodium sulfate and concentrated. The residue was purified by column chromatography (SiO₂, petroleum ether/ethyl acetate=15/1 to 3/1) to give 4-bromobut-2-yn-1-ol (10.9 g, 73.2 mmol, 48.5% yield) as an off-white liquid, characterized by ¹HNMR (EC7119-4-P1N).

¹H NMR (400 MHz, CDCl₃) δ=4.31 (s, 2H), 3.94 (t, J=2.0 Hz, 2H).

### Step 2: 4-bromobut-2-yn-1-yl Nonyl Hydrogen Phosphate (EC7119-6)

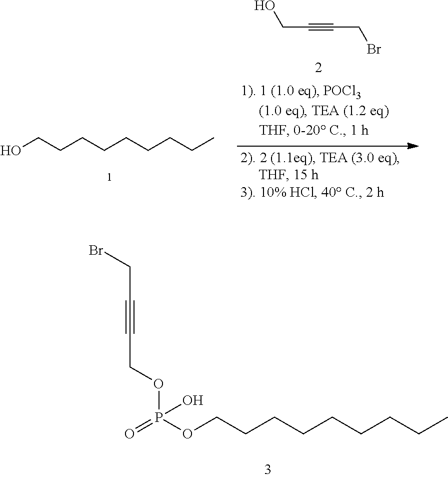

To a solution of POCl₃ (2.13 g, 13.9 mmol, 1.29 mL, 1.0 eq.) in THF (30 mL) was added TEA (1.68 g, 16.6 mmol, 2.32 mL, 1.2 eq.) slowly at 0° C., then nonan-1-ol (2.00 g, 13.9 mmol, 1.0 eq.) dissolved in TIFF (30 mL) was added dropwise. After that, the resulting solution was warmed up to 20° C. and stirred for 1 h. Then the solution was cooled down to 0° C. after the alcohol (Reactant 1) was consumed completely, and a second portion TEA (4.21 g, 41.6 mmol, 5.79 mL, 3.0 eq.) was added followed by 4-bromobut-2-yn-1-ol (2.27 g, 15.3 mmol, 1.1 eq.) in TIFF (30 mL). After that, the reaction mixture was stirred at 20° C. for 15 h. After completion, the reaction was quenched with 1M HCl solution (60 mL), then the solution was heated to 40° C. and stirred for 2 h. After that, the solution was cooled down to 20° C. and extracted with ethyl acetate (100 mL*3) and the organic layers were dried over anhydrous sodium sulfate, filtered and concentrated under reduced pressure. The residue was purified by column chromatography (SiO₂, CH₂Cl₂:MeOH=100/1 to 10/1) to give 4-bromobut-2-yn-1-yl nonyl hydrogen phosphate (800 mg, 2.25 mmol, 16.2% yield) as an off-white oil, characterized by ¹H NMR (EC7119-6-P1N2).

¹H NMR (400 MHz, CDCl₃) δ=4.79-4.54 (m, 1H), 4.31-3.74 (m, 4H), 3.50-3.51 (m, 1H), 1.75-1.55 (m, 2H), 1.29-1.30 (m, 12H), 0.96-0.82 (m, 3H).

### Step 3: 4-(dioctylamino)but-2-yn-1-yl Nonyl Hydrogen Phosphate (EC7119-7)

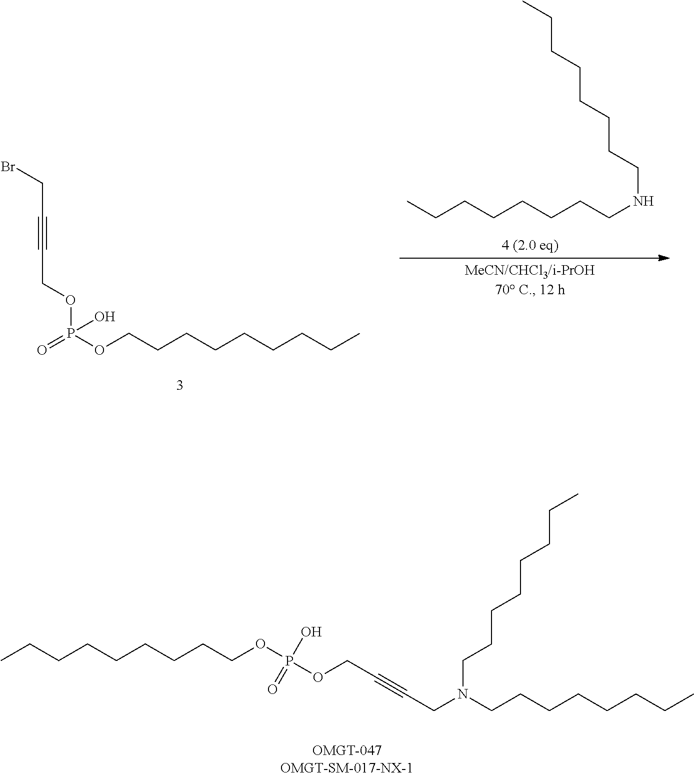 [SMILES: CCCCCCCCCOP(=O)(O)OCC#CCBr ; CCCCCCCCNCCCCCCCC ; CCCCCCCCCOP(=O)(O)OCC#CCN(CCCCCCCC)CCCCCCCC]

To a solution of 4-bromobut-2-yn-1-yl nonyl hydrogen phosphate (800 mg, 2.25 mmol, 1.0 eq.) in MeCN (2.0 mL), i-PrOH (2.0 mL) and CHCl₃ (2.0 mL) was added dioctylamine (1.09 g, 4.50 mmol, 2.0 eq.). Then the mixture was stirred at 70° C. for 12 h under nitrogen atmosphere. After completion, the reaction mixture was concentrated under reduced pressure to give a residue. The residue was purified by column chromatography (SiO₂, CH₂Cl₂:MeOH:NH₃·H₂O=50/1/0.05 to 6/1/0.05) to give 4-(dioctylamino)but-2-yn-1-yl nonyl hydrogen phosphate aka SM-017 (852.46 mg, 1.65 mmol, 73.3% yield, 99.82% purity) as a yellow oil, characterized by ¹HNMR (EC7119-7-P1N), Special analysis (EC7119-7-P1C1) and LCMS (EC7119-7-P1C2).

LCMS: [M+H]⁺: 516.7

¹H NMR (400 MHz, MeOH) δ=4.59-4.48 (m, 2H), 3.90-3.78 (m, 4H), 2.99-2.72 (m, 4H), 1.68-1.55 (m, 6H), 1.40-1.27 (m, 32H), 0.95-0.86 (m, 9H).

### Example 8: Synthesis of 4-(dioctylamino)butyl (3-propylhexyl) Hydrogen Phosphate (OMGT-043, Aka SM-018)

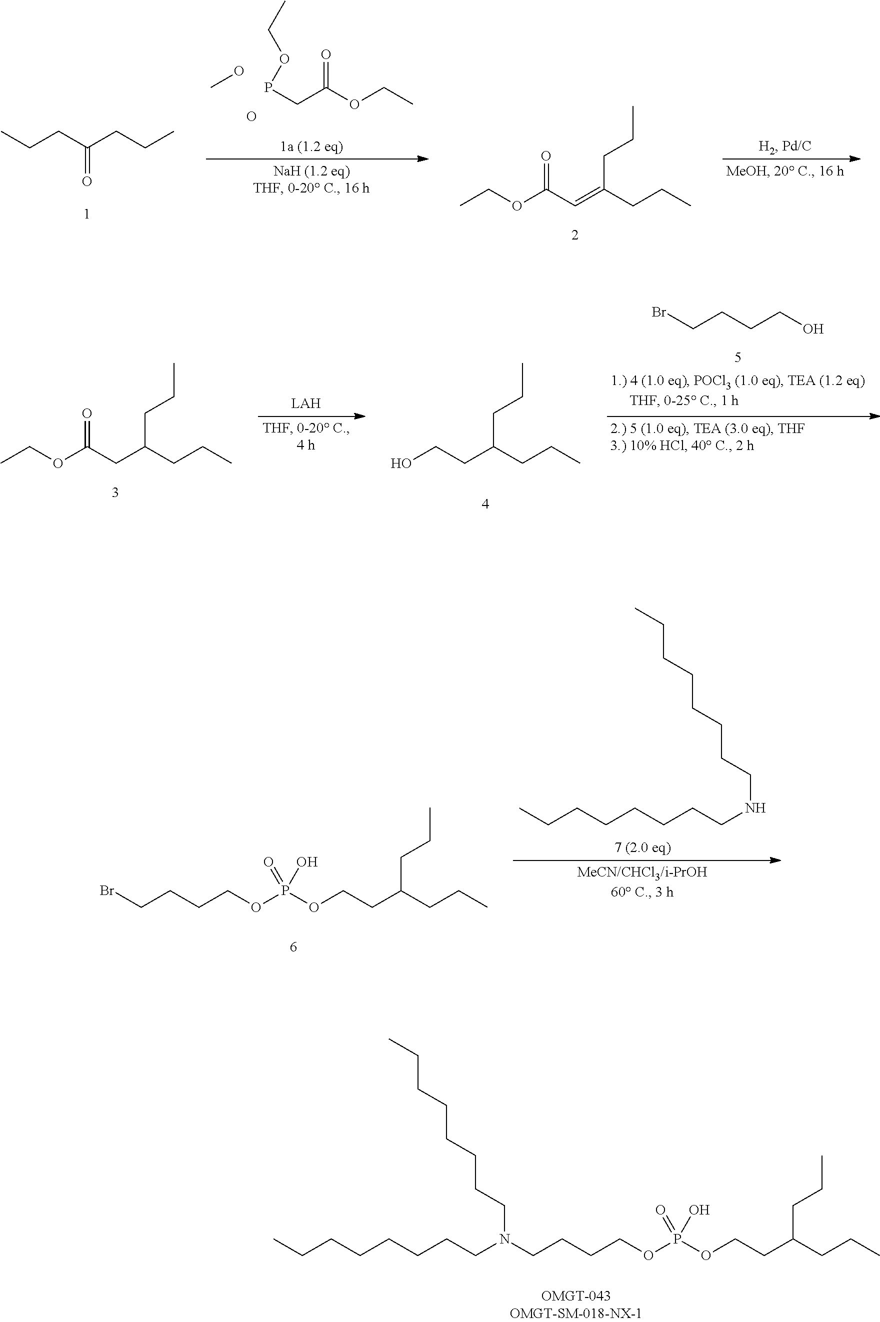

### Step 1: Ethyl 3-propylhex-2-enoate (2): (EC5059-149/152)

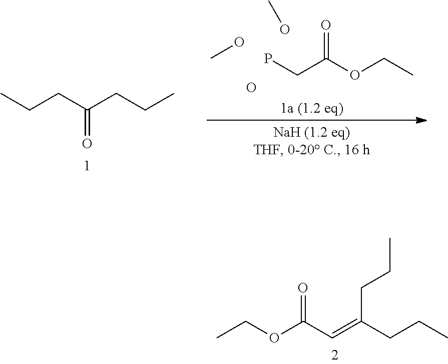

A solution of ethyl 2-diethoxyphosphorylacetate (25.45 g, 113.50 mmol, 22.5 mL, 1.2 eq) in TIF (80 mL) was added dropwise to a suspension of NaH (4.54 g, 113.50 mmol, 60% purity, 1.2 eq) in THF (120 mL) at 0° C. under N₂ over 0.5 h. After addition, the mixture was stirred at 20° C. for 0.5 h, and then heptan-4-one (10.8 g, 94.58 mmol, 13.3 mL, 1 eq) in THF (50 mL) was added dropwise at 0° C. The resulting mixture was stirred at 20° C. for 15 h. The reaction mixture was quenched by addition NH₄Cl (100 mL) at 0° C., and then diluted with EtOAc (100 mL) and extracted with EtOAc (100 mL*3). The combined organic layers were washed with brine (120 mL), dried over Na₂SO₄, filtered and concentrated under reduced pressure to give a residue. The residue was purified by flash silica gel chromatography (120 g SepaFlash® Silica Flash Column, PE:EtOAc: 0˜5%) to give compound ethyl 3-propylhex-2-enoate (7.5 g, 40.70 mmol, 43.1% yield) as colorless liquid.

¹H NMR (400 MHz, CDCl₃)=5.62 (s, 1H), 4.20-4.06 (m, 2H), 2.62-2.53 (m, 2H), 2.11 (t, J=7.2 Hz, 2H), 1.55-1.45 (m, 4H), 1.27 (t, J=7.2 Hz, 3H), 0.98-0.90 (m, 6H).

### Step 2: Ethyl 3-propylhexanoate (3): (EC5059-155)

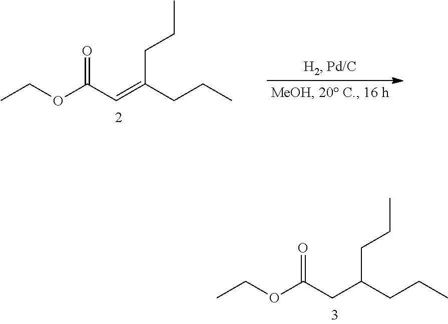

To a solution of ethyl 3-propylhex-2-enoate (6 g, 32.56 mmol, 1 eq) in MeOH (120 mL) was added Pd/C (600 mg, 10% purity) under N₂. The suspension was degassed under vacuum and purged with H₂ several times. The mixture was stirred under H₂ (15 psi) at 20° C. for 16 h. The reaction mixture was filtered and the filter cake was washed with MeOH (20 mL*2). The filtrate was concentrated under reduced pressure to give compound ethyl 3-propylhexanoate (4.9 g, 26.30 mmol, 80.8% yield) as colorless liquid. The crude product was used into the next step without further purification.

¹H NMR (400 MHz, CDCl₃) δ=4.13 (q, J=6.8 Hz, 2H), 2.22 (d, J=7.2 Hz, 2H), 1.92-1.80 (m, 1H), 1.33-1.22 (m, 11H), 0.92-0.86 (m, 6H).

### Step 3: 3-propylhexan-1-ol (4): (EC5059-158/162)

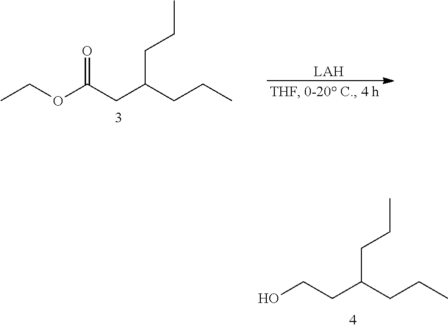

To a solution of ethyl 3-propylhexanoate (4.9 g, 26.30 mmol, 1 eq) in THF (100 mL) was added LAH (1.20 g, 31.56 mmol, 1.2 eq) in portions at 0° C. under N₂. After addition, the mixture was stirred at 20° C. for 4 h. The reaction mixture was quenched by addition H₂O (1.2 mL) at 0° C., and then successively added 15% aq. NaOH (1.2 mL), H₂O (3.6 mL). The reaction mixture was diluted with EtOAc (100 mL) and filtered, the filter cake was washed with EtOAc (30 mL*2). The filtrate was concentrated under reduced pressure to give a residue. The residue was purified by flash silica gel chromatography (40 g SepaFlash® Silica Flash Column, PE:EtOAc: 0˜25%) to give compound 3-propylhexan-1-ol (2.8 g, 19.41 mmol, 73.7% yield) as colorless oil.

¹H NMR (400 MHz, CDCl₃) δ=3.67 (t, J=7.2 Hz, 2H), 1.54-1.51 (m, 2H), 1.48-1.43 (m, 1H), 1.33-1.21 (m, 8H), 0.89 (t, J=6.8 Hz, 6H).

### Step 4: 4-bromobutyl (3-propylhexyl) Hydrogen Phosphate (6): (EC5059-167/171)

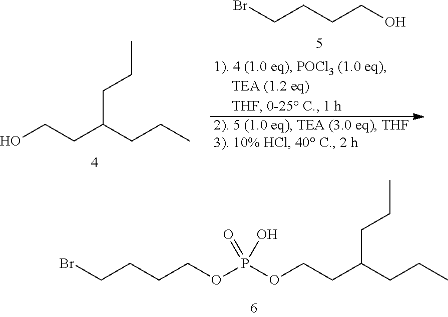

TEA (2.36 g, 23.29 mmol, 3.24 mL, 1.2 eq) was slowly added to POCl₃ (2.98 g, 19.41 mmol, 1.80 mL, 1 eq) in dry THF (50 mL) at 0° C. under N₂. Then 3-propylhexan-1-ol (2.8 g, 19.41 mmol, 1 eq) in THF (30 mL) was added dropwise over 1 h and the resulting mixture was warmed to 20° C. was stirred for 1 h. When all the alcohol had reacted (checked by TLC), the mixture was cooled to 0° C. and a second portion of TEA (5.89 g, 58.23 mmol, 8.1 mL, 3 eq) was added, followed by 4-bromobutan-1-ol (2.97 g, 19.41 mmol, 1 eq) in THF (30 mL). The reaction mixture was stirred at 20° C. for 14 h. Decomposed with HCl 10% (50 mL) and heated at 40° C. for 2 h. THF was removed under vacuum and the aqueous residue was extracted with DCM (150 mL*3). The organic layer was dried over Na₂SO₄, filtered and concentrated under reduced pressure to give a residue. The residue was purified by flash silica gel chromatography (40 g SepaFlash® Silica Flash Column, PE:EtOAc: 0˜50%) and prep-HPLC (MS directive, column: Waters xbridge 150*25 mm 10 um; mobile phase: [water(FA)-ACN], B %: 50%-80%, 8 min) to give compound 4-bromobutyl 3-propylhexyl hydrogen phosphate (1.4 g, 3.81 mmol, 19.8% yield, 97.7% purity) as yellow oil.

LCMS: [2M+H]⁺: 719.0

¹H NMR (400 MHz, CDCl₃) δ=9.86 (brs, 1H), 4.11-4.02 (m, 4H), 3.45 (t, J=6.8 Hz, 2H), 2.06-1.95 (m, 2H), 1.90-1.81 (m, 2H), 1.65 (q, J=6.8 Hz, 2H), 1.51-1.47 (m, 1H), 1.33-1.23 (m, 8H), 0.89 (t, J=6.8 Hz, 6H).

### Step 5: 4-(dioctylamino)butyl (3-propylhexyl) hydrogen phosphate (SM-018): (EC5059-179/181)

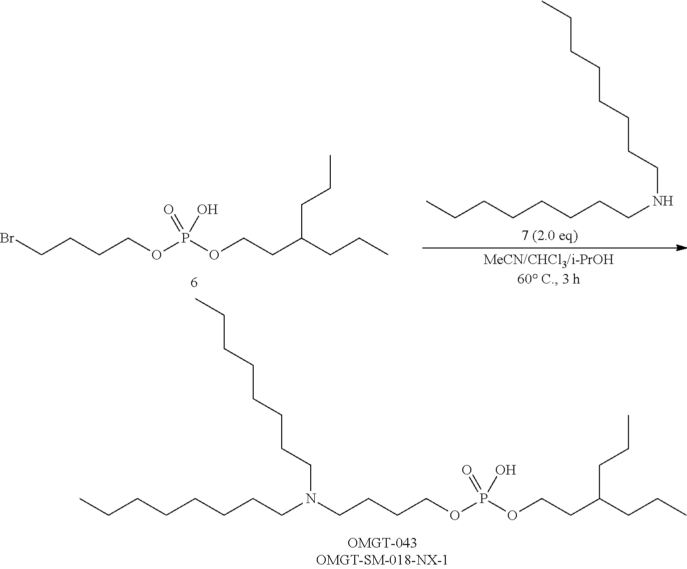 [SMILES: CCCC(CCC)CCOP(=O)(O)OCCCCBr ; CCCCCCCCNCCCCCCCC ; CCCCCCCCN(CCCCCCCC)CCCCOP(=O)(O)OCCC(CCC)CCC]

A mixture of N-octyloctan-1-amine (1.34 g, 5.57 mmol, 2 eq), 4-bromobutyl 3-propylhexyl hydrogen phosphate (1 g, 2.78 mmol, 1 eq) in MeCN (0.5 mL), CHCl₃ (0.5 mL) and i-PrOH (0.5 mL) was degassed and purged with N₂ for 3 times, and then the mixture was stirred at 60° C. for 3 h under N₂ atmosphere. The reaction mixture was directly concentrated under reduced pressure to give a residue. The residue was diluted with DCM (80 mL) and washed with HCl solution (10%, 20 mL*2), dried over Na₂SO₄, filtered and concentrated under reduced pressure to give a residue. The residue was purified by flash silica gel chromatography (40 g SepaFlash® Silica Flash Column, (DCM:MeOH: 0˜5%, 2% NH₃·H₂O in MeOH) to give 4-(dioctylamino)butyl (3-propylhexyl) hydrogen phosphate (SM-018) (588 mg, 1.13 mmol, 42.0% yield, 99.99% purity) as colorless oil.

LCMS: [M+H]⁺: 520.7

¹H NMR (400 MHz, CD₃OD-d₄) δ=3.92-3.85 (m, 4H), 3.20-3.06 (m, 6H), 1.87-1.80 (m, 2H), 1.75-1.66 (m, 6H), 1.62-1.58 (m, 2H), 1.55-1.50 (m, 1H), 1.40-1.28 (m, 28H), 0.94-0.88 (m, 12H).

### Example 9: Synthesis of 4-(dioctylamino)butyl 7-methyloctyl Hydrogen Phosphate (OMGT-042, aka SM-020)

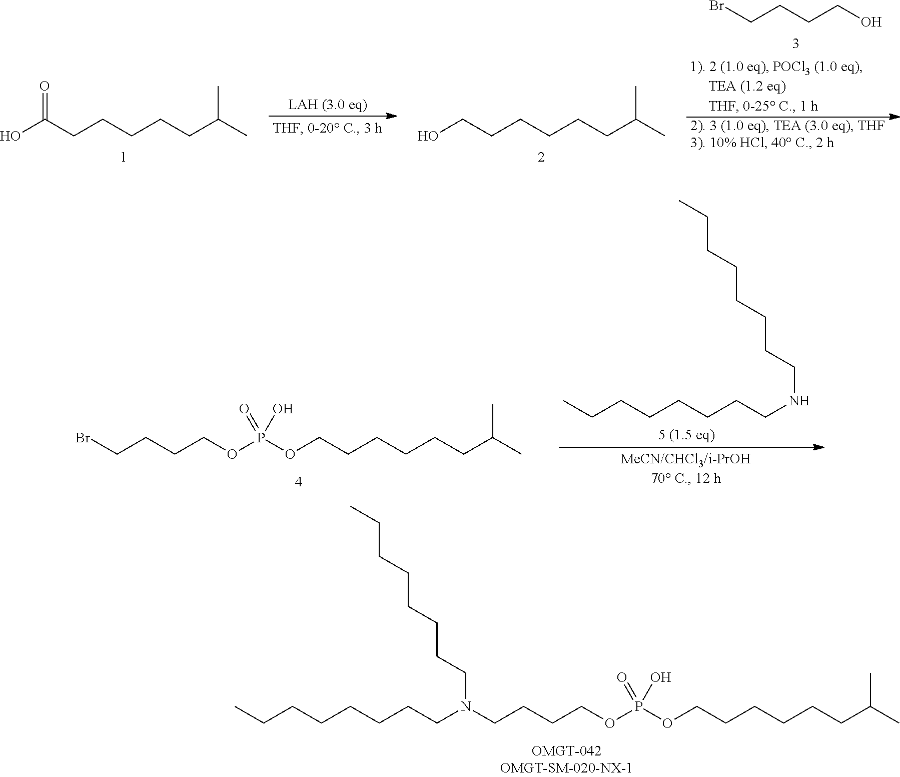

### Step 1: 7-methyloctan-1-ol (2): (EC5000-142)

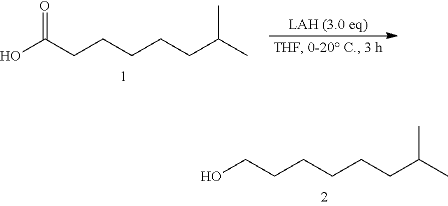

To a solution of 7-methyloctanoic acid (3 g, 18.96 mmol, 1 eq) in THF (50 mL) was added LAH (2.16 g, 56.88 mmol, 3 eq) at 0° C. The mixture was stirred at 20° C. for 2 h. The reaction was quenched with water (2 mL), NaOH (2 mL) and water (6 mL) at 0° C. The reaction mixture was filtered and the filtrate was concentrated under reduced pressure to give a residue. The residue was purified by column chromatography (SiO₂, PE/EtOAc=30/1 to 10/1) to give compound 7-methyloctan-1-ol (2.6 g, 18.02 mmol, 95.1% yield) as a yellow oil.

¹H NMR (400 MHz, CDCl₃) δ=3.67 (t, J=6.8 Hz, 2H), 1.81-1.63 (m, 2H), 1.53-1.42 (m, 1H), 1.31-1.32 (m, 1H), 1.21-1.13 (m, 1H), 1.08-0.89 (m, 12H).

### Step 2: 4-bromobutyl 7-methyloctyl Hydrogen Phosphate (4): (EC5000-151/159/161)

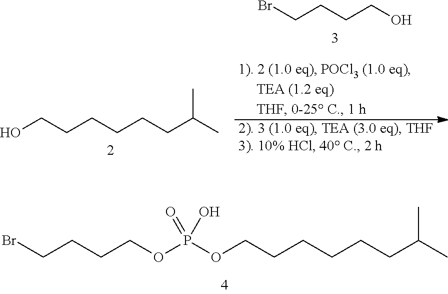

TEA (2.10 g, 20.80 mmol, 2.89 mL, 1.2 eq) was slowly added to a solution of POCl₃ (2.66 g, 17.33 mmol, 1.61 mL, 1 eq) in dry THF (80 mL) at 0° C. under N₂. Then 7-methyloctan-1-ol (2.5 g, 17.33 mmol, 1 eq) in THF (80 mL) was added dropwise over 1 h and the resulting mixture was warmed to 20° C. was stirred for 1 hour. When all the alcohol had reacted (checked by TLC), the mixture was cooled to 0° C. and a second portion of TEA (5.26 g, 51.99 mmol, 7.24 mL, 3 eq) was added, followed by 4-bromobutan-1-ol (2.65 g, 17.33 mmol, 1 eq) in THF (80 mL) was added dropwise. The reaction mixture was stirred at 20° C. for 14 h. Decomposed with HCl 10% (100 mL) and heated at 40° C. for 2 h. THF was removed under vacuum and the aqueous residue was extracted with EtOAc (200 mL×3). The organic layer was dried over Na₂SO₄, filtered, and the filtrate was concentrated under reduced pressure to give a residue. The residue was purified by column chromatography (SiO₂, DCM/MeOH=40/1 to 20/1) and prep-HPLC (column: Waters xbridge 150×25 mm 10 um; mobile phase: [water(FA)-ACN]; B %: 50%-80%, 8 min) to give compound 4-bromobutyl 7-methyloctyl hydrogen phosphate (700 mg, 1.75 mmol, 24.2% yield, 90% purity) as a yellow oil.

¹H NMR (400 MHz, CDCl₃) δ=7.72-7.55 (m, 1H), 4.23-3.92 (m, 4H), 3.45 (t, J=6.4 Hz, 2H), 2.18-1.96 (m, 2H), 1.92-1.81 (m, 2H), 1.74-1.69 (m, 2H), 1.59-1.47 (m, 1H), 1.38-1.04 (m, 4H), 0.99-0.89 (m, 10H).

### Step 3: 4-(dioctylamino)butyl 7-methyloctyl Hydrogen Phosphate: (EC5000-163/168/169)

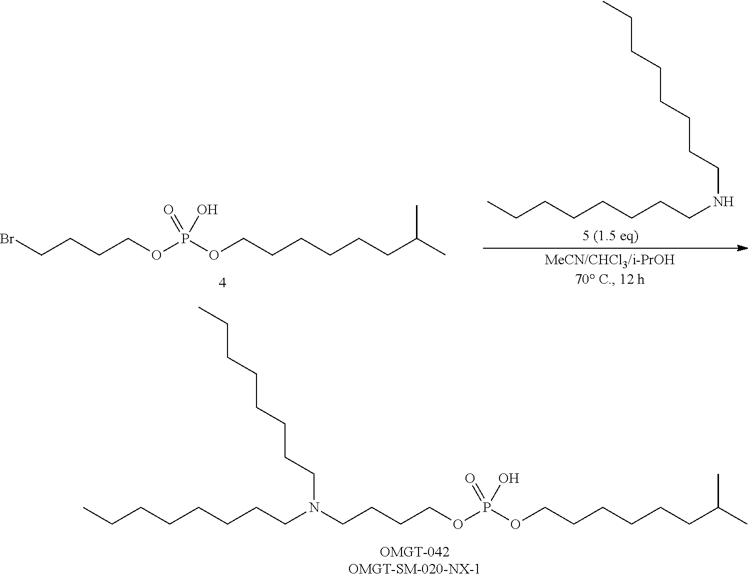 [SMILES: CC(C)CCCCCCOP(=O)(O)OCCCCBr ; CCCCCCCCNCCCCCCCC ; CCCCCCCCN(CCCCCCCC)CCCCOP(=O)(O)OCCCCCCC(C)C]

To a solution of 4-bromobutyl 7-methyloctyl hydrogen phosphate (700 mg, 1.95 mmol, 1 eq) in MeCN (1 mL), CHCl₃ (1 mL) and i-PrOH (1 mL) was added N-octyloctan-1-amine (705.74 mg, 2.92 mmol, 1.5 eq). The mixture was stirred at 70° C. for 12 h. The reaction mixture was concentrated under reduced pressure to give a residue. The residue was diluted with MeCN (30 mL) and washed with HCl solution (30 mL×2), dried over Na₂SO₄, filtered and concentrated under reduced pressure to give a residue. The residue was purified by column chromatography (SiO₂, DCM/MeOH=40/1 to 10/1) and then by prep-HPLC (column: Waters xbridge 150×25 mm 10 um; mobile phase: [water(FA)-MeOH]; B %: 80%-100%, 8 min) to give compound 4-(dioctylamino)butyl 7-methyloctyl hydrogen phosphate aka SM-020 (231 mg, 444.42 umol, 39.8% yield, 100% purity) as an off-white gum.

LCMS: [M+H]⁺: 520.6

¹H NMR (400 MHz, CD₃OD) δ=3.95-3.81 (m, 4H), 3.23-3.07 (m, 6H), 1.89-1.80 (m, 2H), 1.77-1.64 (m, 8H), 1.57-1.18 (m, 24H), 1.15-1.06 (m, 1H), 0.97-0.89 (m, 16H).

### Example 10: Synthesis of 2-butylhexyl 4-(dioctylamino)butyl Hydrogen Phosphate (OMGT-044, Aka SM-021)

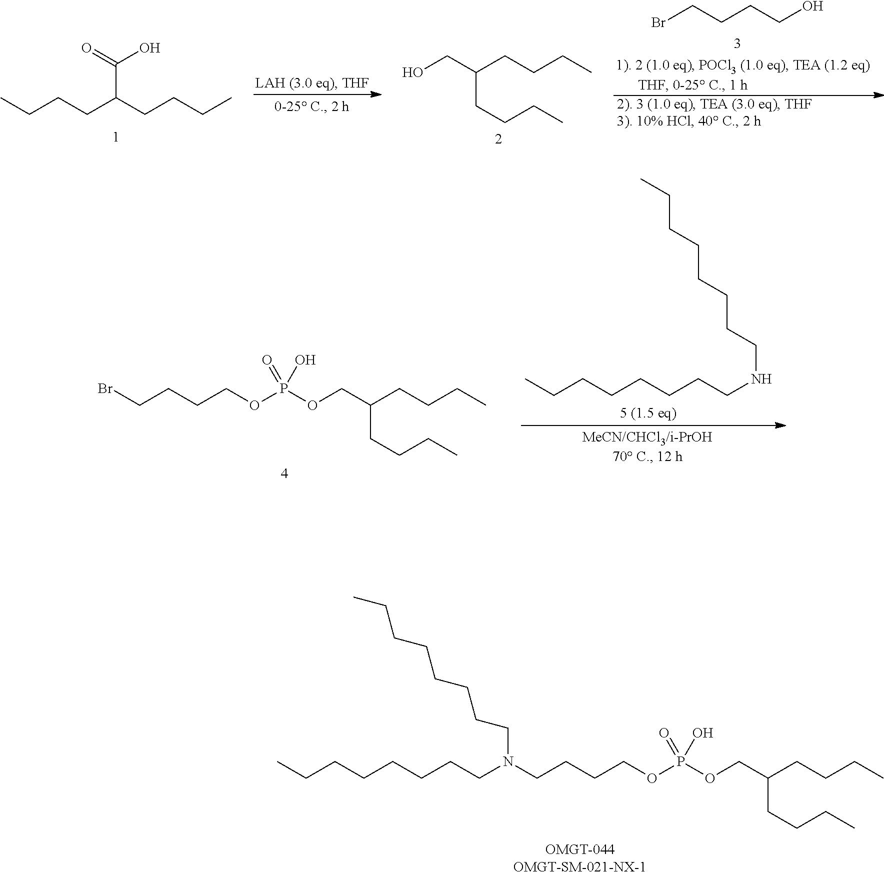

### Step 1: 2-butylhexan-1-ol (2): (EC5000-141)

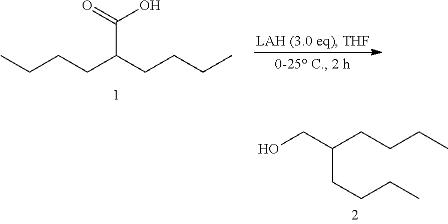

To a solution of 2-butylhexanoic acid (14 g, 81.27 mmol, 1 eq) in THF (150 mL) was added LAH (9.25 g, 243.81 mmol, 3 eq) at 0° C. The mixture was stirred at 25° C. for 4 h. The reaction was quenched with water (10 mL), NaOH (10 mL) and water (30 mL) at 0° C. The reaction mixture was filtered and the filtrate was concentrated under reduced pressure to give a residue. The residue was purified by column chromatography (SiO₂, PE/EtOAc=40/1 to 20/1) to give compound 2-butylhexan-1-ol (10.7 g, 67.60 mmol, 83.2% yield) as a yellow oil.

¹H NMR (400 MHz, CDCl₃) δ=3.51 (d, J=5.2 Hz, 2H), 1.48-1.39 (m, 1H), 1.34-1.23 (m, 12H), 1.03-0.75 (m, 6H).

### Step 2: 4-bromobutyl 7-methyloctyl Hydrogen Phosphate: (EC5000-145/149)

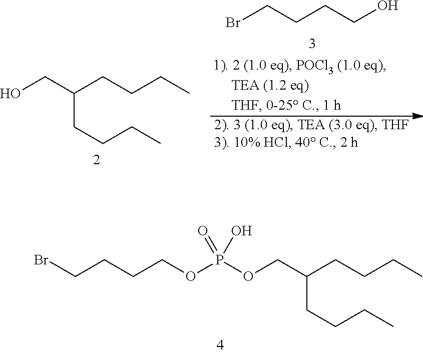

TEA (1.53 g, 15.16 mmol, 2.11 mL, 1.2 eq) was slowly added to a solution of POCl₃ (1.94 g, 12.64 mmol, 1.17 mL, 1 eq) in dry THF (80 mL) at 0° C. under N₂. Then 2-butylhexan-1-ol (2 g, 12.64 mmol, 1 eq) in THF (80 mL) was added dropwise over 1 h and the resulting mixture was warmed to 20° C. was stirred for 1 h. When all the alcohol had reacted (checked by TLC), the mixture was cooled to 0° C. and a second portion of TEA (3.84 g, 37.91 mmol, 5.28 mL, 3 eq) was added, followed by 4-bromobutan-1-ol (1.93 g, 12.64 mmol, 1 eq) in THF (80 mL). The reaction mixture was stirred at 20° C. for 14 h. Decomposed with HCl 10% (100 mL) and heated at 40° C. for 2 h. THF was removed under vacuum and the aqueous residue was extracted with EtOAc (200 mL×3). The organic layer was dried over Na₂SO₄, filtered, reduced under vacuum. The residue was purified by column chromatography (SiO₂, DCM/MeOH=40/1 to 20/1) to give compound 4-bromobutyl 2-butylhexyl hydrogen phosphate (1.6 g, 4.29 mmol, 40.0% yield) as a yellow oil.

¹H NMR (400 MHz, CDCl₃) δ=4.18-3.86 (m, 4H), 3.62-3.21 (m, 2H), 2.13-1.77 (m, 4H), 1.65-1.56 (m, 1H), 1.41-1.23 (m, 12H), 0.96-0.84 (m, 6H).

### Step 3: 2-butylhexyl 4-(dioctylamino)butyl Hydrogen Phosphate: (EC5000-158/167)

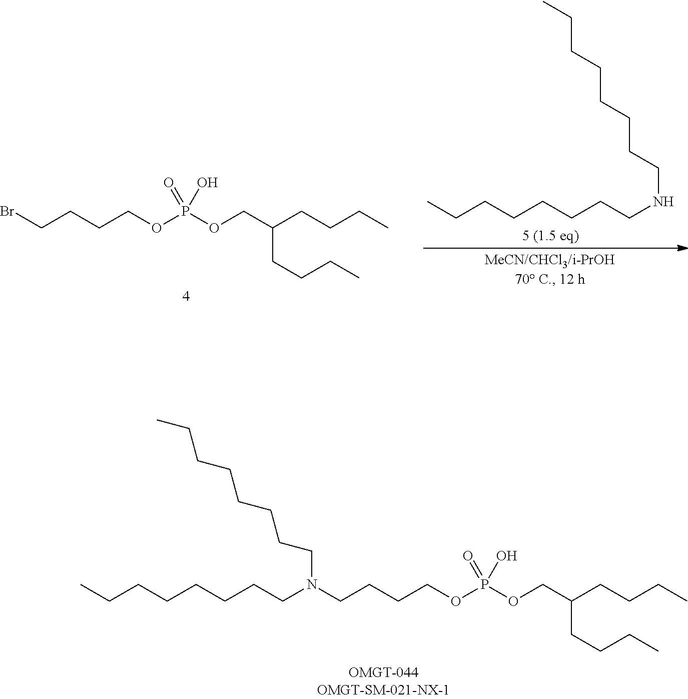 [SMILES: CCCCC(CCCC)COP(=O)(O)OCCCCBr ; CCCCCCCCNCCCCCCCC ; CCCCCCCCN(CCCCCCCC)CCCCOP(=O)(O)OCC(CCCC)CCCC]

To a solution of 4-bromobutyl 2-butylhexyl hydrogen phosphate (1.6 g, 4.29 mmol, 1 eq) in MeCN (1 mL), CHCl₃ (1 mL) and i-PrOH (1 mL) was added N-octyloctan-1-amine (1.55 g, 6.43 mmol, 1.5 eq). The mixture was stirred at 70° C. for 12 h. The reaction mixture was concentrated under reduced pressure to give a residue. The residue was diluted with MeCN (30 mL) and washed with HCl solution (0.5 N, 30 mL×2), dried over Na₂SO₄, filtered and concentrated under reduced pressure to give a residue. The residue was purified by column chromatography (SiO₂, DCM/MeOH=40/1 to 10/1) and then by prep-HPLC (column: Waters xbridge 150×25 mm 10 um, mobile phase: [water (FA)-MeOH], B %: 80%-100%, 8 min) to give compound 2-butylhexyl 4-(dioctylamino)butyl hydrogen phosphate aka SM-021 (440 mg, 824.27 umol, 44.0% yield, 100% purity) as a colorless oil.

LCMS: [M+H]⁺: 534.6

¹H NMR (400 MHz, CD₃OD) δ=3.95-3.81 (m, 2H), 3.79-3.63 (m, 2H), 3.25-3.00 (m, 6H), 1.95-1.79 (m, 2H), 1.72-1.62 (m, 6H), 1.59-1.49 (m, 1H), 1.42-1.25 (m, 32H), 0.95-0.81 (m, 12H).

### Example 11: Synthesis of (E)-4-(dioctylamino)but-2-en-1-yl Nonyl Hydrogen Phosphate (OMGT-045, Aka SM-022)

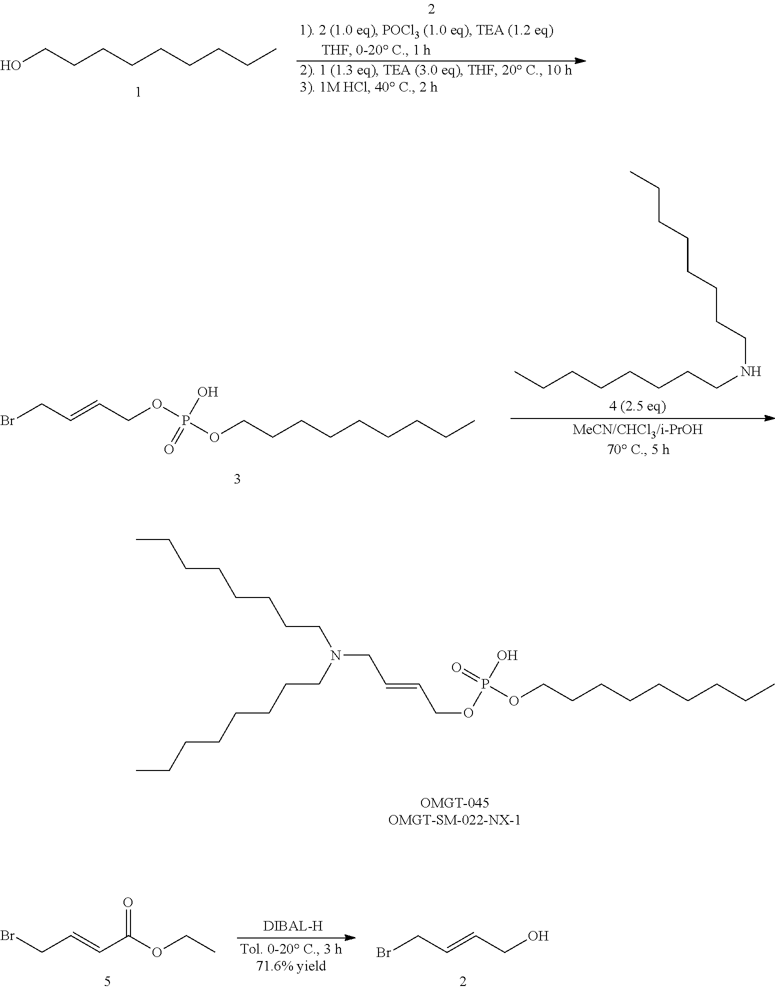

### Step 1: (E)-4-bromobut-2-en-1-ol (EC7197-1)

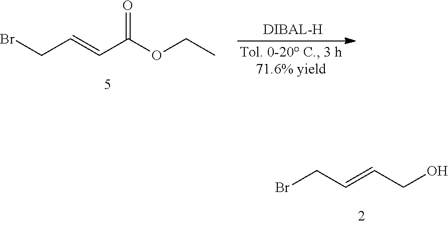

To a solution of ethyl (E)-4-bromobut-2-enoate (10.0 g, 51.8 mmol, 7.14 mL, 1.0 eq.) in toluene (120 mL) was added DIBAL-H (1 M, 109 mL, 2.1 eq.) dropwise at 0° C. over 1 h. Then the mixture was stirred at 0° C. for 2 h under nitrogen atmosphere. After completion, the mixture was quenched with 2N HCl solution (30 mL) at 0° C. and stirred for 30 minutes. Then the solution was diluted with ethyl acetate. The organic was washed with brine, dried with anhydrous sodium sulfate and concentrated. The residue was purified by column chromatography (SiO₂, petroleum ether/ethyl acetate=30/1 to 3/1) to give (E)-4-bromobut-2-en-1-ol (5.60 g, 37.1 mmol, 71.6% yield) as a colorless oil, characterized by ¹HNMR (EC7197-1-P1N1).

¹H NMR (400 MHz, CDCl₃) δ=5.96-5.92 (m, 2H), 4.19 (d, J=3.6 Hz, 2H), 4.00-3.96 (m, 2H)

### Step 2: (E)-4-bromobut-2-en-1-yl Nonyl Hydrogen Phosphate (EC7197-5)

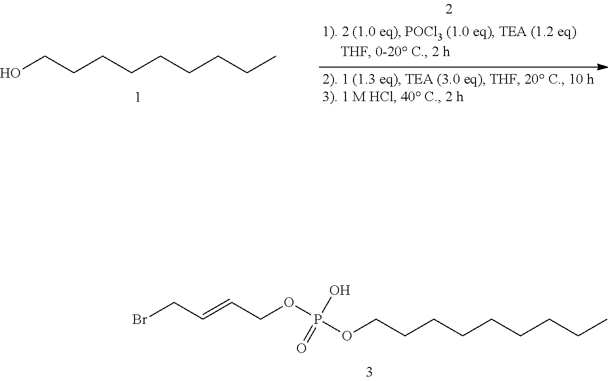

To a solution of POCl₃ (2.13 g, 13.9 mmol, 1.29 mL, 1.0 eq.) in THF (80 mL) was added TEA (1.68 g, 16.6 mmol, 2.32 mL, 1.2 eq.) slowly at 0° C., then nonan-1-ol (2.00 g, 13.9 mmol, 1.0 eq.) dissolved in THF (50 mL) was added dropwise. After that, the resulting solution was warmed up to 20° C. and stirred for 2 h. Then the solution was cooled down to 0° C. after the alcohol (Reactant 1) was consumed completely, and a second portion TEA (4.21 g, 41.6 mmol, 5.79 mL, 3.0 eq.) was added, followed by (E)-4-bromobut-2-en-1-ol (2.72 g, 18.0 mmol, 1.3 eq.) in THF (50 mL). After that, the reaction mixture was stirred at 20° C. for 10 h. After completion, the reaction was quenched with 1M HCl solution (20 mL), then the solution was heated to 40° C. and stirred for 2 h. After that, the solution was cooled down to 20° C. and extracted with ethyl acetate (200 mL*3) and the organic layers were dried over anhydrous sodium sulfate, filtered and concentrated under reduced pressure. The residue was purified by column chromatography (SiO₂, CH₂Cl₂:MeOH=100/1 to 10/1) to give (E)-4-bromobut-2-en-1-yl nonyl hydrogen phosphate (1.4 g, crude) as a yellow oil, characterized by ¹HNMR (EC7197-5-P1N3).

¹H NMR (400 MHz, CDCl₃) δ=6.02-5.84 (m, 2H), 4.16-4.12 (m, 2H), 4.09-4.02 (m, 2H), 3.95-3.91 (m, 2H), 1.61-1.59 (m, 2H), 1.34-1.30 (m, 12H), 0.88-0.86 (m, 3H).

### Step 3: (E)-4-(dioctylamino)but-2-en-1-yl Nonyl Hydrogen Phosphate (EC7197-7)

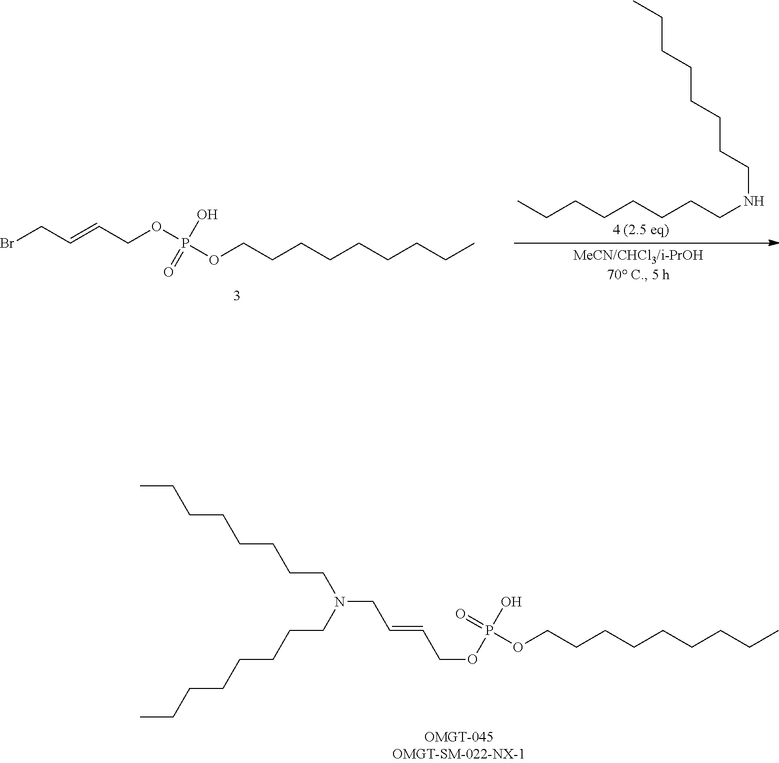

To a solution of (E)-4-bromobut-2-en-1-yl nonyl hydrogen phosphate (800 mg, 2.24 mmol, 1.0 eq.), i-PrOH (1.5 mL) and CHCl₃ (1.5 mL) in MeCN (1.5 mL) was added dioctylamine (1.35 g, 5.60 mmol, 2.5 eq.). Then the mixture was stirred at 70° C. for 5 h under nitrogen atmosphere. After completion, the reaction mixture was concentrated under reduced pressure. The residue was diluted with MeCN (20 mL) and filtered. The filtrate was concentrated under reduced pressure to give a residue. The residue was purified by column chromatography (SiO₂, CH₂Cl₂/MeOH/NH₃·H₂O=200/1/0.05 to 6/1/0.05) and then prep-HPLC (FA condition; column: Waters xbridge 150*25 mm 10 um; mobile phase: [water (FA)-MeOH]; B %: 80%-100%, 8 min) to give (E)-4-(dioctylamino)but-2-en-1-yl nonyl hydrogen phosphate aka SM-022 (217 mg, 420 umol, 18.8% yield, 99.9% purity) as a white gum, characterized by ¹HNMR (EC7197-7-P1B1), Special HPLC (EC7197-7-P1B1) and LCMS (EC7197-7-P1B1).

LCMS: [M+H]⁺: 518.5

¹H NMR (400 MHz, CDCl₃) δ=6.15-5.93 (m, 2H), 4.49-4.38 (m, 2H), 3.92-3.81 (m, 2H), 3.53 (d, J=6.8 Hz, 2H), 3.03-2.82 (m, 4H), 1.70-1.57 (m, 6H), 1.35-1.22 (m, 32H), 0.94-0.83 (m, 9H).

### Example 12: Synthesis of (Z)-4-(dioctylamino)butyl non-3-en-1-yl Hydrogen Phosphate (OMGT-040, Aka SM-023)

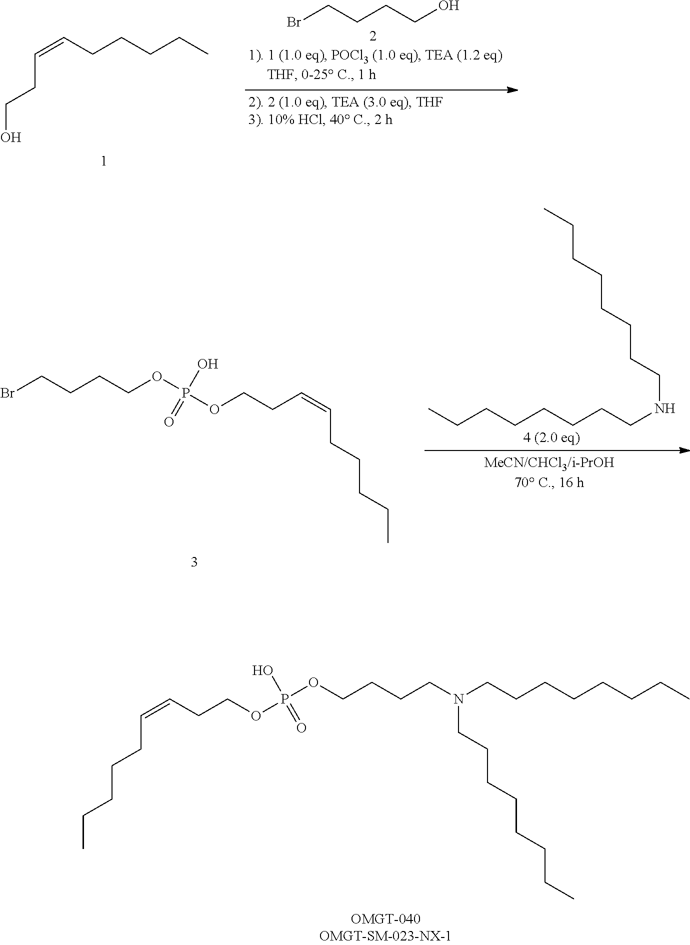

### Step 1: (Z)-4-bromobutyl non-3-en-1-yl hydrogen phosphate: (EC5500-72)

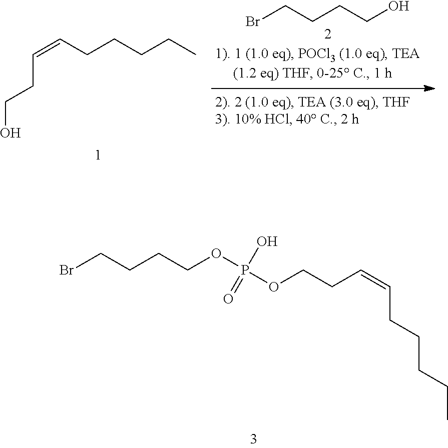

To a solution of TEA (8.54 g, 84.37 mmol, 11.74 mL, 1.2 eq) in dry THF (200 mL) was slowly added POCl₃ (10.78 g, 70.30 mmol, 6.53 mL, 1 eq) at 0° C. under N₂. Then the solution of (Z)-non-3-en-1-ol (10.00 g, 70.30 mmol, 1 eq) in THF (150 mL) was added dropwise over 1 h and the resulting mixture was warmed to 20° C. stirring for 1 h. When all the alcohol had reacted (checked by TLC), the mixture was cooled to 0° C. and a second portion of TEA (21.34 g, 210.91 mmol, 29.36 mL, 3 eq) was added, followed by 4-bromobutan-1-ol (10.76 g, 70.30 mmol, 1 eq) in THF (50 mL). The reaction mixture was stirred at 20° C. for 14 h. The reaction mixture was decomposed with HCl (10%, 300 mL) and heated at 40° C. for 2 h. THF was removed under vacuum and the aqueous residue was extracted with DCM (300 mL×3). The organic layer was dried over Na₂SO₄, filtered, and concentrated under reduced pressure. The residue was purified by flash silica gel chromatography (ISCO®; 80 g SepaFlash® Silica Flash Column, Eluent of 0˜10% MeOH/DCM gradient @ 80 mL/min). Compound 4-bromobutyl [(Z)-non-3-enyl]hydrogen phosphate (20.00 g, 55.99 mmol, 79.6% yield) was obtained as a yellow oil.

¹H NMR (400 MHz, CDCl₃) δ=5.65-5.25 (m, 2H), 4.18-3.65 (m, 4H), 3.53-3.38 (m, 2H), 2.56-2.35 (m, 2H), 2.05-1.69 (m, 6H), 1.45-1.20 (m, 6H), 0.90 (t, J=6.8 Hz, 3H).

### Step 2: (Z)-4-(dioctylamino)butyl non-3-en-1-yl hydrogen phosphate: (EC5500-75/80)

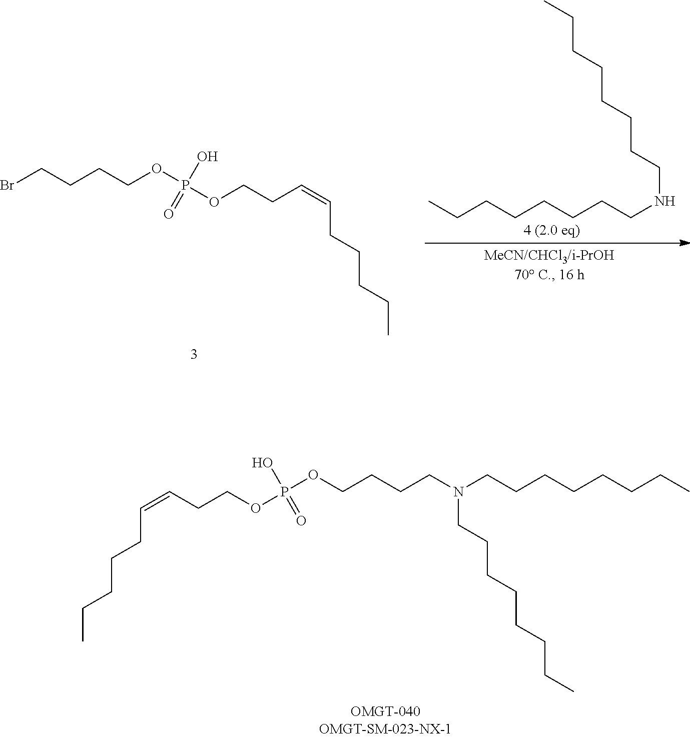 [SMILES: CCCCC/C=C\CCOP(=O)(O)OCCCCBr ; CCCCCCCCNCCCCCCCC ; CCCCC/C=C\CCOP(=O)(O)OCCCCN(CCCCCCCC)CCCCCCCC]

To a mixture of 4-bromobutyl [(Z)-non-3-enyl]hydrogen phosphate (3.00 g, 8.40 mmol, 1 eq) in MeCN (2 mL), CHCl₃ (2 mL) and i-PrOH (2 mL) was added N-octyloctan-1-amine (4.06 g, 16.80 mmol, 2.0 eq), the reaction mixture was stirred at 70° C. for 16 h. After the reaction was completed, the reaction mixture was directly concentrated under reduced pressure to give a residue. The residue was diluted with DCM (50 mL) and washed with HCl solution (10%, 50 mL×2), dried over Na₂SO₄, filtered and concentrated under reduced pressure to give a residue. The residue was purified by flash silica gel chromatography (ISCO®; 80 g SepaFlash® Silica Flash Column, Eluent of 0˜10% MeOH/DCM gradient @80 mL/min) to yield compound 4-(dioctylamino)butyl [(Z)-non-2-enyl]hydrogen phosphate or (Z)-4-(dioctylamino)butyl non-3-en-1-yl hydrogen phosphate aka SM-023 (900.00 mg, 772.56 umol, 33.3% yield, 99.99% purity) was obtained as a yellow oil.

LCMS: [M+H]⁺: 518.5

¹H NMR (400 MHz, CDCl₃) δ=13.38-12.51 (brs, 1H), 5.52-5.34 (m, 2H), 3.98-3.84 (m, 4H), 3.06-2.81 (m, 6H), 2.45-2.35 (m, 2H), 2.06-1.91 (m, 4H), 1.76-1.60 (m, 6H), 1.36-1.23 (m, 26H), 1.01-0.70 (m, 9H).

### Example 13: Synthesis of (Z)-4-(dioctylamino)but-2-en-1-yl Nonyl Hydrogen Phosphate (OMGT-046, aka SM-024)

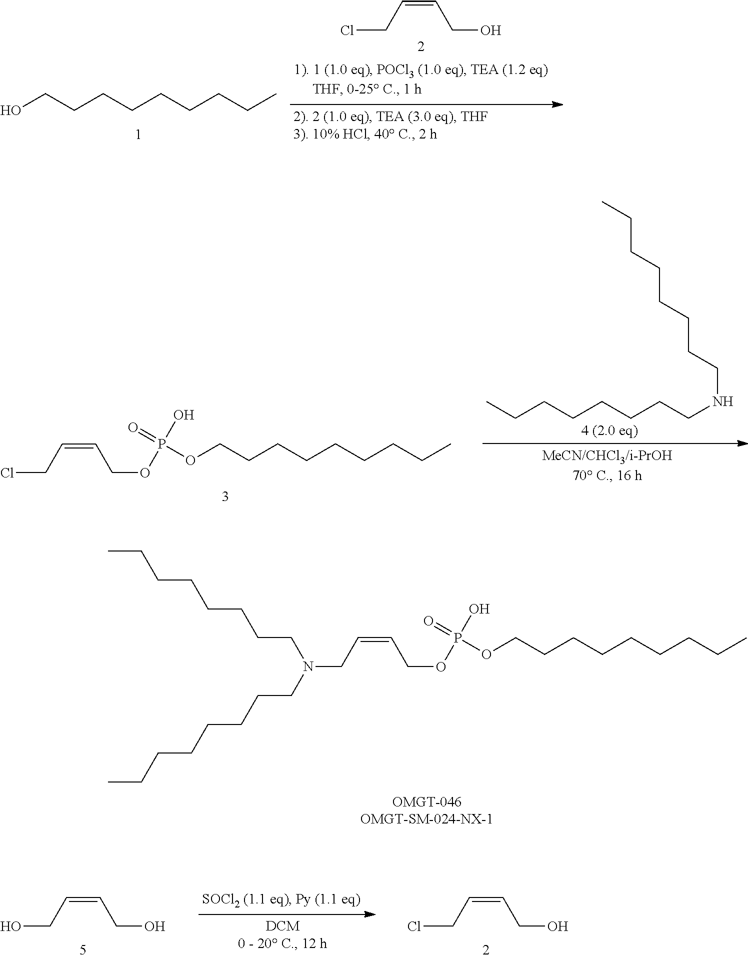

### Step 1: (Z)-4-chlorobut-2-en-1-ol: (EC5500-84/85)

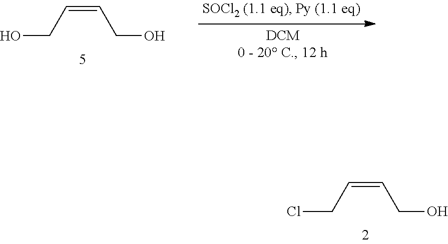 [SMILES: OC/C=C\CO ; OC/C=C\CCl]

To a mixture of (Z)-but-2-ene-1,4-diol (30.00 g, 340.50 mmol, 28.04 mL, 1 eq) and PYRIDINE (29.63 g, 374.55 mmol, 30.23 mL, 1.1 eq) in DCM (30 mL) was added SOCl₂ (44.56 g, 374.55 mmol, 27.17 mL, 1.1 eq) dropwise at 0° C., then the reaction mixture was heated to 25° C. and stirred for 12 h. The reaction mixture was poured into ice water (50 mL) and extracted with EtOAc (50 mL×3), the combined organic layers were washed with NaHCO₃ solution (100 mL), dried over Na₂SO₄ and concentrated under reduced pressure. The residue was purified by flash silica gel chromatography (ISCO®; 80 g SepaFlash® Silica Flash Column, Eluent of 0˜30% EtOAc/PE gradient @100 mL/min). Compound (Z)-4-chlorobut-2-en-1-ol (10.00 g, 93.85 mmol, 50.0% yield) was obtained as a yellow liquid.

¹H NMR (400 MHz, CDCl₃) δ=5.87-5.74 (m, 2H), 4.30 (d, J=5.6 Hz, 2H), 4.14 (d, J=7.2 Hz, 2H).

### Step 2: (Z)-4-chlorobut-2-en-1-yl Nonyl Hydrogen Phosphate: (EC5500-86/87)

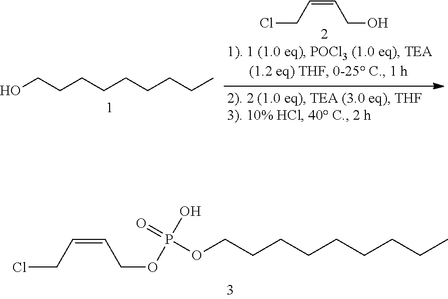

To a solution of TEA (841.76 mg, 8.32 mmol, 1.16 mL, 1.2 eq) in dry THF (20 mL) was slowly added POCl₃ (1.06 g, 6.93 mmol, 644.20 uL, 1 eq) at 0° C. under N₂. Then nonan-1-ol (1.00 g, 6.93 mmol, 1 eq) in THF (15 mL) was added dropwise over 1 h and the resulting mixture was warmed to 20° C. stirring for 1 h. When all the alcohol had reacted (checked by TLC), the reaction mixture was cooled to 0° C. and a second portion of TEA (2.10 g, 20.80 mmol, 2.89 mL, 3 eq) was added to, followed by (Z)-4-chlorobut-2-en-1-ol (738.63 mg, 6.93 mmol, 1 eq) in THF (5 mL). The reaction mixture was stirred at 20° C. for 14 h. The reaction mixture was decomposed with HCl (10%, 30 mL) and heated at 40° C. for 2 h. THF was removed under vacuum and the aqueous residue was extracted with DCM (30 mL×3). The organic layer was dried over Na₂SO₄, filtered, and concentrated under reduced pressure. The residue was purified by flash silica gel chromatography (ISCO®; 20 g SepaFlash® Silica Flash Column, Eluent of 0˜10% MeOH/DCM gradient @ 40 mL/min). Compound [(Z)-4-chlorobut-2-enyl]nonyl hydrogen phosphate (728.00 mg, 2.33 mmol, 72.8% yield) was obtained as a yellow oil.

¹H NMR (400 MHz, CDCl₃) δ=5.89-5.81 (m, 2H), 4.72-4.58 (m, 2H), 4.12 (d, J=7.2 Hz, 2H), 4.03 (q, J=6.8 Hz, 2H), 1.76-1.62 (m, 2H), 1.40-1.20 (m, 12H), 0.89 (t, J=6.4 Hz, 3H).

### Step 3: (Z)-4-(dioctylamino)but-2-en-1-yl Nonyl Hydrogen Phosphate: (EC5500-89/90)

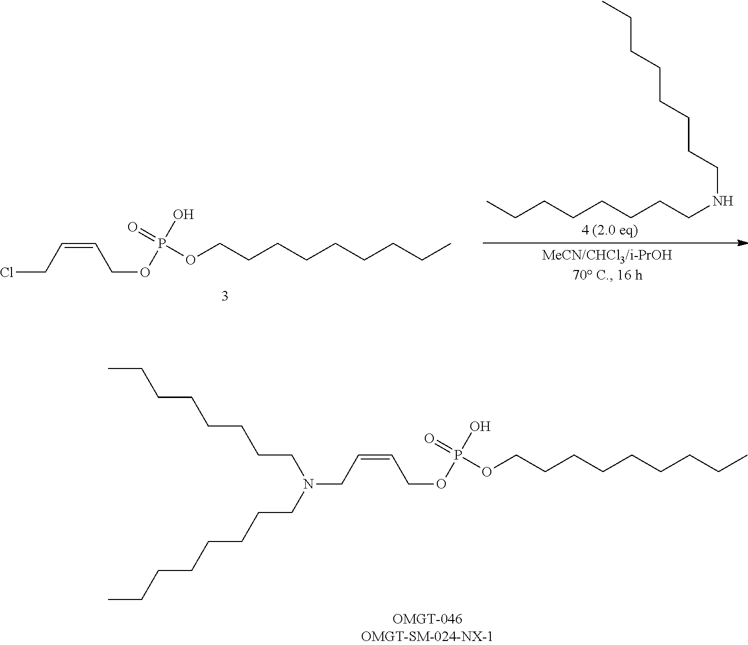 [SMILES: CCCCCCCCCOP(=O)(O)OC/C=C\CCl ; CCCCCCCCNCCCCCCCC ; CCCCCCCCCOP(=O)(O)OC/C=C\CN(CCCCCCCC)CCCCCCCC]

To a solution of [(Z)-4-chlorobut-2-enyl]nonyl hydrogen phosphate (728.00 mg, 2.33 mmol, 1 eq) and N-octyloctan-1-amine (1.12 g, 4.66 mmol, 2.0 eq) in MeCN (0.5 mL), CHCl₃ (0.5 mL) and i-PrOH (0.5 mL) was added N-octyloctan-1-amine (1.12 g, 4.66 mmol, 2.0 eq), The reaction mixture was stirred at 70° C. for 12 h. The reaction mixture was directly concentrated under reduced pressure to obtain a residue. The residue was purified by flash silica gel chromatography (ISCO®; 20 g SepaFlash® Silica Flash Column, Eluent of 0˜10% MeOH/DCM gradient @ 40 mL/min). Compound [(Z)-4-(dioctylamino)but-2-enyl]nonyl hydrogen phosphate or ((Z)-4-(dioctylamino)but-2-en-1-yl nonyl hydrogen phosphate aka SM-024 (500.00 mg, 927.07 umol, 48.0% yield) was obtained as a yellow oil.

LCMS: [M+H]⁺: 518.40

¹H NMR (400 MHz, CDCl₃) δ=13.93-13.35 (m, 1H), 6.25-6.09 (m, 1H), 5.72-5.58 (m, 1H), 4.63-4.51 (m, 2H), 3.91 (q, J=6.7 Hz, 2H), 3.68 (br d, J=8.1 Hz, 2H), 3.06-2.78 (m, 4H), 1.79-1.57 (m, 6H), 1.42-1.19 (m, 32H), 0.88 (qd, J=3.5, 6.8 Hz, 9H).

### Example 14: Synthesis of 5-(dioctylamino)pentyl Nonyl Hydrogen Phosphate (OMGT-054, Aka SM-026)

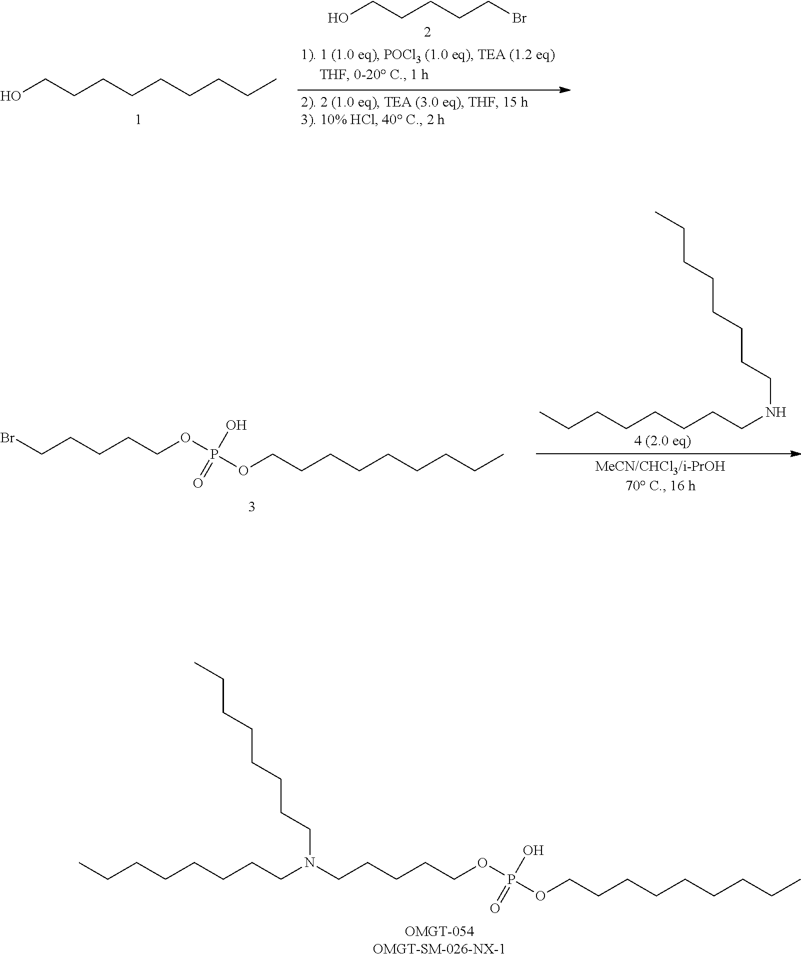

### Step 1: 5-bromopentyl Nonyl Hydrogen Phosphate (EC7119-29)

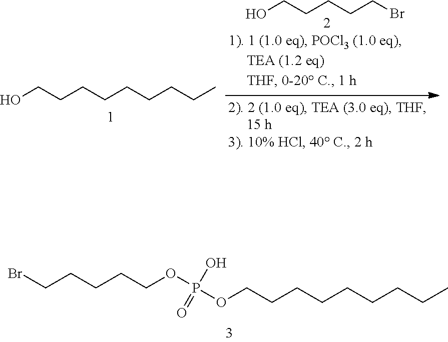

To a solution of POCl₃ (10.6 g, 69.3 mmol, 6.44 mL, 1.0 eq.) in THF (50 mL) was added TEA (8.42 g, 83.2 mmol, 11.6 mL, 1.2 eq.) slowly at 0° C., then nonan-1-ol (10.0 g, 69.3 mmol, 1.0 eq.) dissolved in THF (50 mL) was added dropwise. After that, the resulting solution was warmed up to 20° C. and stirred for 1 h. Then the solution was cooled down to 0° C. after the alcohol (Reactant 1) was consumed completely, and a second portion TEA (21.0 g, 208 mmol, 29.0 mL, 3.0 eq.) was added, followed by 5-bromopentan-1-ol (11.6 g, 69.3 mmol, 1.0 eq.) in THF (50 mL). After that, the reaction mixture was stirred at 20° C. for 15 h. After completion, the reaction was quenched with 1M HCl solution (100 mL), then the solution was heated to 40° C. and stirred for 2 h. After that, the solution was cooled down to 20° C. and extracted with ethyl acetate (150 mL*3) and the organic layers were dried over anhydrous sodium sulfate, filtered and concentrated under reduced pressure. The residue was purified by column chromatography (SiO₂, CH₂Cl₂:MeOH=50/1 to 5/1) to give 5-bromopentyl nonyl hydrogen phosphate (3.6 g, 7.25 mmol, 10.5% yield, 75.2% purity) as a yellow oil, characterized by HNMR (EC7119-29-P1N4) and LCMS (EC7119-29-P1C₂).

LCMS: [M+H]⁺: 373.0

¹H NMR (400 MHz, CDCl₃) δ=4.19-3.92 (m, 4H), 3.41 (t, J=6.4 Hz, 2H), 1.97-1.81 (m, 2H), 1.74-1.58 (m, 4H), 1.56-1.46 (m, 2H), 1.36-1.22 (m, 12H), 0.89 (t, J=6.6 Hz, 3H).

### Step 2: 5-(dioctylamino)pentyl Nonyl Hydrogen Phosphate (EC7119-36)

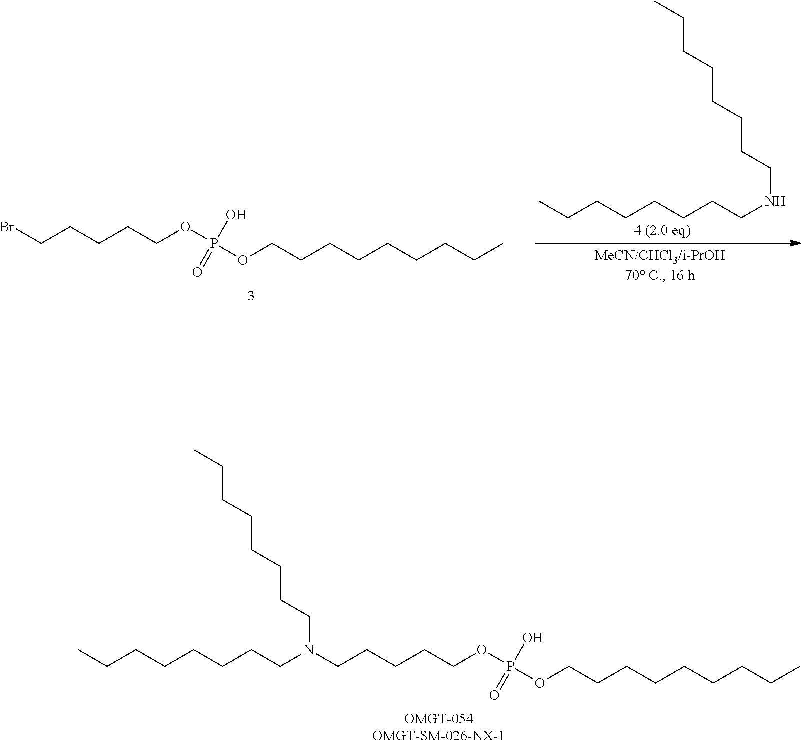

To a solution of 5-bromopentyl nonyl hydrogen phosphate (3.10 g, 8.31 mmol, 1.0 eq.) in MeCN (5.0 mL), i-PrOH (5.0 mL) and CHCl₃ (5.0 mL) was added dioctylamine (4.01 g, 16.6 mmol, 2.0 eq.). Then the mixture was stirred at 70° C. for 16 h under nitrogen atmosphere. After completion, the reaction mixture was concentrated under reduced pressure to give a residue. The residue was purified by column chromatography (SiO₂, CH₂Cl₂:MeOH:NH₃·H₂O=50/1/0.05 to 5/1/0.05) to give 5-(dioctylamino)pentyl nonyl hydrogen phosphate aka SM-026 (549.70 mg, 1.03 mmol, 12.4% yield, 99.87% purity) as a yellow oil, characterized by ¹HNMR (EC7119-38-P1N4), LCMS (EC7119-38-P1B1) and Special Analysis (EC7119-38-P1B2).

LCMS: [M+H]⁺: 534.9

¹H NMR (400 MHz, CD₃OD) δ=3.93-3.76 (m, 4H), 3.12-2.96 (m, 6H), 1.75-1.59 (m, 10H), 1.54-1.48 (m, 2H), 1.42-1.27 (m, 32H), 1.03-0.78 (m, 9H).

### Example 15: Synthesis of 6-(dioctylamino)hexyl Nonyl Hydrogen Phosphate (OMGT-055, Aka SM-027)

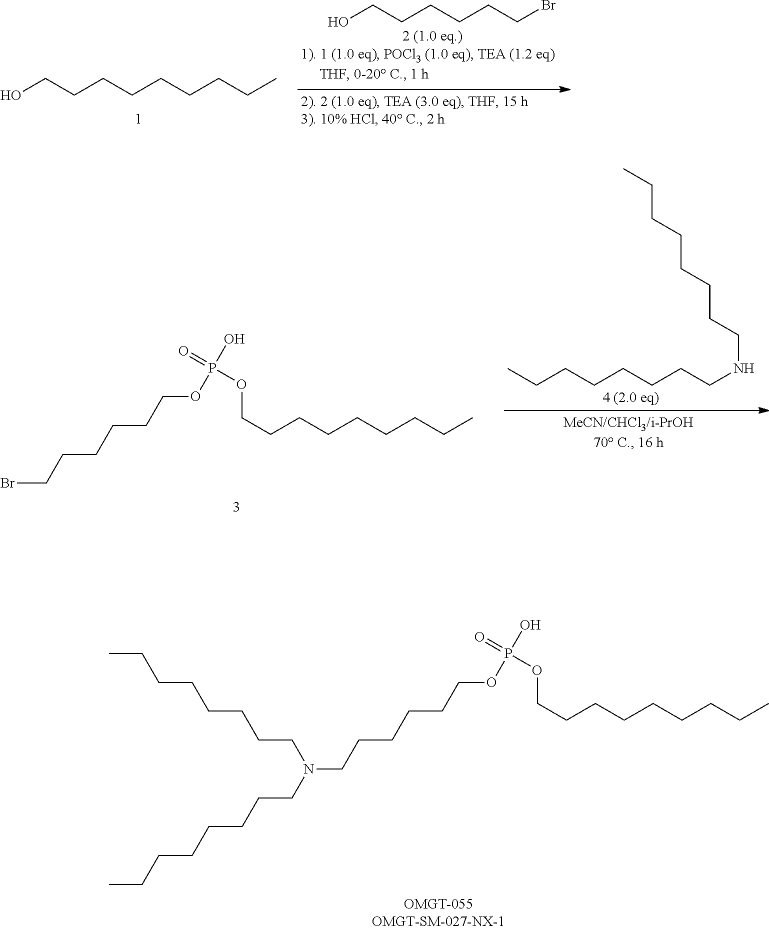

### Step 1: 5-bromopentyl Nonyl Hydrogen Phosphate (EC7119-31)

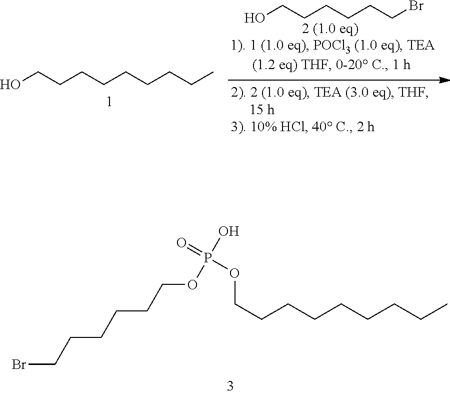

To a solution of POCl₃ (10.6 g, 69.3 mmol, 6.44 mL, 1.0 eq.) in THF (50 mL) was added TEA (8.42 g, 83.2 mmol, 11.6 mL, 1.2 eq.) slowly at 0° C., then nonan-1-ol (10.0 g, 69.3 mmol, 1.0 eq.) dissolved in TIFF (50 mL) was added dropwise. After that, the resulting solution was warmed up to 20° C. and stirred for 1 h. Then the solution was cooled down to 0° C. after the alcohol (Reactant 1) was consumed completely, and a second portion TEA (21.0 g, 208 mmol, 29.0 mL, 3.0 eq.) was added, followed by 6-bromohexan-1-ol (12.6 g, 69.3 mmol, 9.10 mL, 1.0 eq.) in THF (50 mL). After that, the reaction mixture was stirred at 20° C. for 15 h. After completion, the reaction was quenched with 1M HCl solution (100 mL), then the solution was heated to 40° C. and stirred for 2 h. After that, the solution was cooled down to 20° C. and extracted with ethyl acetate (150 mL*3) and the organic layers were dried over anhydrous sodium sulfate, filtered and concentrated under reduced pressure. The residue was purified by column chromatography (SiO₂, CH₂Cl₂:MeOH=50/1 to 8/1) to give 6-bromohexyl nonyl hydrogen phosphate (14.0 g, 36.2 mmol, 70.0% yield) as a yellow oil, characterized by HNMR (EC7119-34-P1N1).

¹H NMR (400 MHz, CDCl₃) δ=4.17-3.98 (m, 4H), 3.41 (t, J=6.8 Hz, 2H), 1.92-1.82 (m, 2H), 1.77-1.69 (m, 4H), 1.53-1.41 (m, 4H), 1.40-1.35 (m, 2H), 1.33-1.24 (m, 10H), 0.93-0.83 (m, 3H).

### Step 2: 6-(dioctylamino)hexyl nonyl hydrogen phosphate (EC7119-35)

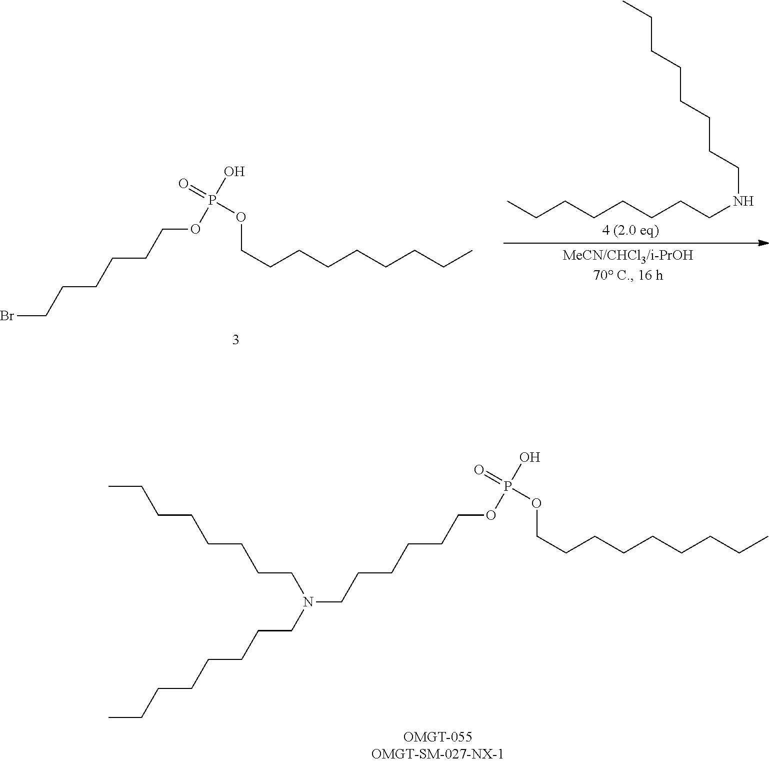

To a solution of 6-bromohexyl nonyl hydrogen phosphate (2.00 g, 5.16 mmol, 1.0 eq.) in MeCN (4.0 mL), i-PrOH (4.0 mL) and CHCl₃ (4.0 mL) was added dioctylamine (2.49 g, 10.3 mmol, 2.0 eq.). Then the mixture was stirred at 70° C. for 16 h under nitrogen atmosphere. After completion, the reaction mixture was concentrated under reduced pressure to give a residue. The residue was purified by column chromatography (SiO₂, CH₂Cl₂:MeOH:NH₃·H₂O=50/1/0.05 to 8/1/0.05) and then by prep-HPLC (column: Phenomenex luna C18 150*25 mm*10 um; mobile phase: [water (FA)-MeOH]; B %: 70%-100%, 8 min) to give 6-(dioctylamino)hexyl nonyl hydrogen phosphate aka SM-027 (615.22 mg, 1.12 mmol, 43.8% yield, 99.59% purity) as a yellow oil, characterized by ¹HNMR (EC7119-37-P1N1), LCMS (EC7119-37-P1B1) and Special HPLC (EC7119-37-P1B2).

LCMS: [M+H]⁺: 548.5

¹H NMR (400 MHz, CD₃OD) δ=3.92-3.77 (m, 4H), 3.18-3.04 (m, 6H), 1.76-1.60 (m, 10H), 1.53-1.44 (m, 4H), 1.42-1.28 (m, 32H), 1.00-0.83 (m, 9H).

### Example 16: Lipid Nanoparticle (LNP) Formulation

Nanoparticles can be formulated using a microfluidic mixer, cross, or a T-junction by the mixing of two or three fluid streams containing nucleic acid cargo and the lipid components respectively.

Lipid components are prepared by combining a lipid according to the formula of 20-30 mol % of cationic lipids (such as DOTAP, DDAB or SM-005 (β-L-arginyl-2,3-diamino propionic acid-N-palmityl-N-oleyl-amide trihydrochloride)), 30 to 50 mol % of phospholipid (such as SM-037), 30 to 50 mol % of a structural lipid (such as cholesterol), and 0.3 to 5 mol % of a PEG-lipid (such as PEG-DMG) at a combined concentrations at about 10 to 50 mM in ethanol. Lipid mixture is diluted with ethanol and water to a final lipid concentration between about 3 and 75 mM.

Nanoparticle compositions including the nucleic acids and lipid components are prepared by rapidly mixing the organic solution containing the lipid components with the aqueous solution of nucleic acid cargo with a total lipid to nucleic acid w/w ratio between about 10:1 and about 100:1 either using a NanoAssemblr microfluidic based system or an equivalent pump system at flow rates between about 8 and about 30 mL/min into the nucleic acid aqueous solution with an aqueous to organic volume ratio between about 1:1 and about 6:1.

The resulting mixture is then immediately diluted with water to a final ethanol concentration between about 10% and 20%. The diluted suspension is buffered exchanged to a storage buffer containing between about 5-15% sugar (such as sucrose or trehalose), 10-100 mM of a NaCl, 10-200 mM Tris-HCL, 10-200 mM Tris-Base, and 10-200 mM sodium acetate between about a pH of 6.5-8.0 and an osmolarity between about 200-400 mOsm/kg. The resulting mixture was then concentrated using a dead-end filtration on a spin column (MilliporeSigma, Amicon) and then sterile filtered using a 0.2 um sterile filter and diluted to a desired concentration between about 0.1 mg/mL and about 2.0 mg/mL nucleic acid prior to storing at temperature at −80° C., −20° C., or at 4° C. The isolated LNPs were characterized to determine the encapsulation efficiency, average hydrodynamic size, and polydispersity index, as described below.

mRNA cargo used here includes but not limited to: FLuc-mRNA (TriLink BioTechnologies).

Cationic lipids includes: 1,2-DiLinoleyloxy-N,N-dimethylaminopropane. (“DLinDMA”), 1,2-Dilinolenyloxy-N,N-dimethylaminopropane (“DLenDMA”), dioctadecyldimethylammonium (“DODMA”), Distearyldimethylammonium (“DSDMA”), N,N-dioleyl-N,N-dimethylammonium chloride (“DODAC”); N-(2,3-dioleyloxy)propyl)-N,N,N-trimethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3-(N—(N′,N′-dimethylaminoethane)-carbamoyl)cholesterol (“DC-Chol”) and N-(1,2-dimyristyloxyprop-3-yl)-N,N-dimethyl-N-hydroxyethyl ammonium bromide (“DMRIE”). For example, cationic lipids that have a positive charge at below physiological pH include, but are not limited to: DODAP, DODMA, DMDMA, and SM-005 (β-L-arginyl-2,3-diamino propionic acid-N-palmityl-N-oleyl-amide trihydrochloride). In some cases, the cationic lipids comprise a protonatable tertiary amine head group, C18 alkyl chains, ether linkages between the head group and alkyl chains, and 0 to 3 double bonds. Such lipids include, e.g., DSDMA, DLinDMA, DLenDMA, and DODMA.

Chemical structure of SM-005 (β-L-arginyl-2,3-diamino propionic acid-N-palmityl-N-oleyl-amide trihydrochloride):

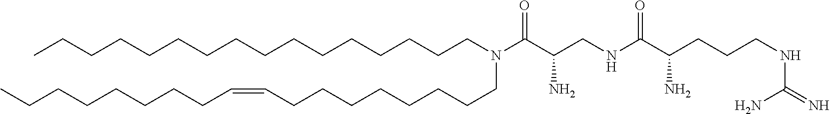 [SMILES: CCCCCCCC/C=C\CCCCCCCCN(CCCCCCCCCCCCCCCC)C(=O)[C@@H](N)CNC(=O)[C@@H](N)CCCNC(=N)N]

“Helper” lipids include:

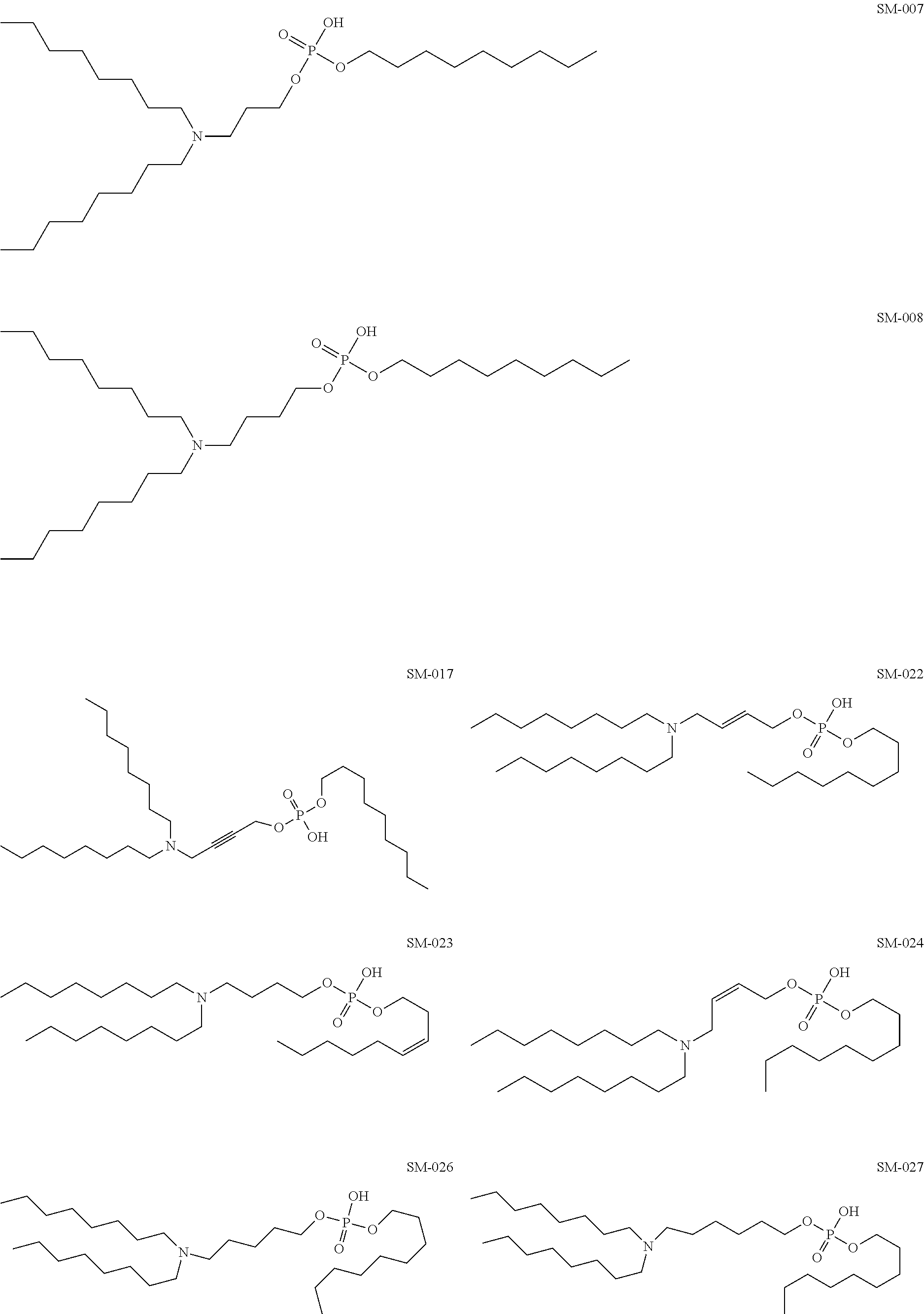

Sterols include, for example, cholesterol.

PEG-lipids includes from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-cholesterol (1-[8′-(Cholest-5-en-3[beta]-oxy)carboxamido-3′,6′-dioxaoctanyl]carbamoyl-[omega]-methyl-poly(ethylene glycol), 1,2-dimyristoyl-sn-glycero-3-phosphoethanolamine-N-[methoxy(polyethylene glycol)-2000](PEG2k-DMG) (cat. #880150P from Avanti Polar Lipids, Alabaster, Alabama, USA), 1,2-distearoyl-sn-glycero-3-phosphoethanolamine-N-[methoxy(polyethylene glycol)-2000](PEG2k-DSPE) (cat. #880120C from Avanti Polar Lipids, Alabaster, Alabama, USA), 1,2-distearoyl-sn-glycerol, methoxypoly ethylene glycol (PEG2k-DSG; GS-020, NOF Tokyo, Japan), poly(ethylene glycol)-2000-dimethacrylate (PEG2k-DMA), 1,2-dioleoyl-sn-glycero-3-phosphoethanolamine-N-[amino(polyethylene glycol)-2000](PEG2K, DOPE), 1,2-Dioleoyl-sn-glycero-3-phosphoethanolamine-polyethylene glycol methoxy (PEG, DOPE 2k, 5k, 20k), and in some embodiments the stealth lipid may be α-Methoxy-ω-(3-oxopropoxy), polyoxyethylene (Methoxy PEG, Aldehyde). In one embodiment, the stealth lipid may be PEG2k-DMG. In some embodiments, the stealth lipid may be PEG2k-DSG. In one embodiment, the stealth lipid may be PEG2k-DSPE. In one embodiment, the stealth lipid may be PEG2K-DOPE. In some embodiments, the stealth lipid may be PEG5k-DOPE. In some embodiments, the stealth lipid may be Methoxy PEG aldehyde 20k. In some embodiments, the stealth lipid may be PEG2K-Cholesterol.

TABLE 1
Lipid composition in LNPs

| | | Mole % ratio |
|---|---|---|
| | Lipid Components | (cationic/helper/ |
| | (cationic/helper/sterol/PEG) | sterol/PEG) |
| LNP1 | DDAB/SM-008/Cholesterol/PEG-DMG | 22.8/38/38/1.2 |
| LNP2 | DDAB/SM-008/DOPE/Cholesterol/ | 22.8/38/7.6/ |
| | PEG-DMG | 30.4/1.2 |
| LNP3 | DOTAP/SM-007/Cholesterol/PEG-DMG | 22.8/38/38/1.2 |
| LNP4 | DOTAP/SM-008/Cholesterol/PEG-DMG | 22.8/38/38/1.2 |
| LNP5 | SM-005/SM-008/Cholesterol/PEG-DMG | 22.8/38/38/1.2 |
| LNP6 | SM-005/SM-017/Cholesterol/PEG-DMG | 22.8/38/38/1.2 |
| LNP7 | SM-005/SM-022/Cholesterol/PEG-DMG | 22.8/38/38/1.2 |
| LNP8 | SM-005/SM-023/Cholesterol/PEG-DMG | 22.8/38/38/1.2 |
| LNP9 | SM-005/SM-024/Cholesterol/PEG-DMG | 22.8/38/38/1.2 |
| LNP10 | SM-026/DSPC/Cholesterol/PEG-DMG | 50/10/38.5/1.5 |
| LNP11 | SM-027/DSPC/Cholesterol/PEG-DMG | 50/10/38.5/1.5 |

### LNP Characterization

A DynaPro® Plate Reader III (Wyatt Technology, Santa Barbara, CA, US) was used to determine the particle size and the polydispersity index (PDI). A Mobius™ (Wyatt Technology, Santa Barbara, CA, US) was used to determine the zeta potential of the nanoparticle compositions. The nanoparticle formulations were diluted 50 to 100-fold in 1× buffer (Tris-HCl or Tris-Acetate buffer, 10-100 mM, pH 7.0-7.5) in determining particle size, PDI, and zeta potential.

A QUANT-IT™ RIBOGREEN® RNA assay (Invitrogen Corporation Carlsbad, Calif.) was used to evaluate the encapsulation of mRNA by the nanoparticle composition. The samples were diluted to a concentration of approximately 0.2-2 μg/ml in a TE buffer solution (10 mM Tris-HCl, 1 mM EDTA, pH 7.5). Diluted samples were transferred to a polystyrene 96 well plate and equivalent volume of either TE buffer or 0.5-200 Triton X-100 solution was added to the wells. The RIBOGREEN® reagent was diluted 1:200 in TE buffer, and 2× volume of this solution was added to each well. The fluorescence intensity was measured using a fluorescence plate reader (Tecan Spark, Tecan Trading AG, Switzerland) at an excitation wavelength of about 485 nm and an emission wavelength of about 530 nm. The fluorescence values of the reagent blank were subtracted from that of each of the samples and the percentage of free mRNA was determined by dividing the fluorescence intensity of the intact sample (without addition of Triton X-100) by the fluorescence value of the disrupted sample (caused by the addition of Triton X-100).

The values for average particle size, polydispersity, and 00 EE are reported in the Table 2 below for various LNP compositions.

TABLE 2
LNP analytical characterization (size,
size distribution and encapsulation)

| | Size (nm) | PDI | % Encapsulation |
|---|---|---|---|
| LNP1 | 107.2 | 0.087 | 88.0 |
| LNP2 | 112.6 | 0.06 | 86.8 |
| LNP3 | 124.3 | 0.115 | 82.5 |
| LNP4 | 130.5 | 0.111 | 92.6 |
| LNP5 | 98.1 | 0.102 | 96.9 |
| LNP6 | 130.1 | 0.138 | 74.0 |
| LNP7 | 105.7 | 0.107 | 89.8 |
| LNP8 | 81.2 | 0.061 | 95.8 |
| LNP9 | 102.4 | 0.119 | 91.9 |
| LNP10 | 83.6 | 0.117 | 11.0 |
| LNP11 | 139.6 | 0.469 | 11.0 |

### Example 17: Comparison of Lipid pKas

The reversible zwitterionic lipids disclosed herein have an ionizable tertiary amine that is connected to an electron withdrawing phosphate group via a ≥C₃ linker configured to increase the pKa of the tertiary amine by increasing the distance between the ionizable tertiary amine and the electron withdrawing phosphate group. Without being bound by theory, it is believed that increasing the length of the linker between the tertiary amine and the phosphate group to ≥C3 increases the pKa of the tertiary amine. To test this, the ChemDraw portal (Perkin Elmer; see the world wide web at (www).perkinelmerinformatics.com/products/research/chemdraw) was used to calculate the predicted pKa of representative lipids SM-007, SM-009, and SM-012, as well as similar compounds 9A1P9, 10A1P9, and 9A1P8 disclosed in Liu et al. (Nat Mater (2021) 20(5): 701-710). The comparison of pKa values for these sets of compounds is shown in Table 3.

TABLE 3
Comparison of Lipid pKas with Prior Art Compounds

| | | Similar Liu | |
|---|---|---|---|
| SM Compound | | Compound | |
| (C3 linker) | pKa | (C2 linker) | pKa |
| SM-007 | 9.541 | 9A1P9 | 8.685 |
| SM-009 | 9.537 | 10A1P9 | 8.664 |
| SM-012 | 9.541 | 9A1P8 | 8.669 |

This data shows that increasing the linker length from C₂ (used in the compounds disclosed in Liu et al.) to C₃, which is used in SM-007, SM-009, and SM-012, results in a significant increase in the predicted pKa value of the tested lipids relative to the compounds disclosed in Liu et al. Advantageously, it was discovered that increasing the pKa of the tertiary amine beneficially impacts its ionization at specific pH with a subsequent increase in the ability of the ionizable lipid to enhance endosomal escape efficiency of a lipid particle(s) into which the ionizable lipid is incorporated. For example, lipid particles or lipid nanoparticles that include reversible zwitterionic lipids as disclosed herein display improved endosomal escape and thereby increased efficiency of delivery of therapeutic agents.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context.

The terms “comprising” and “including” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the techniques herein. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms.

The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the techniques disclosed herein without departing from the scope and spirit of the disclosure. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A pharmaceutical composition comprising a reversible zwitterionic lipid of Formula I having the following structure: 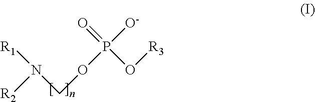
or a salt or isomer thereof, wherein
R₁ and R₂ are either the same or different and are independently C₇-C₂₂ alkyl, C₇-C₂₂ alkenyl, or C₇-C₂₂ alkynyl, optionally R₁, R₂, or R₁ and R₂ are an optionally substituted heterocycle or R₁ and R₂ may join to form an optionally substituted heterocycle;
R₃ is optionally substituted C₃-C₂₂ alkyl, C₃-C₂₂ alkenyl, or C₃-C₂₂ alkynyl; and
n is 3, 4, 5, 6, 7, 8, 9, 10, 11, 12, 13, 14, 15, 16, 17, 18, 19, 20, 21, or 22.

2. The pharmaceutical composition of claim 1, or salt or isomer thereof, wherein R₁ and R₂ are the same.

3. The pharmaceutical composition of claim 1, or salt or isomer thereof, wherein R₁ or R₂ are independently selected from the group consisting of C₇-C₁₈ alkyl, C₇-C₁₈ alkenyl, and C₇-C₁₈ alkynyl, and R₃ is optionally substituted C₇-C₁₈ alkyl, C₇-C₁₈ alkenyl, or C₇-C₁₈ alkynyl, optionally wherein R₁ and R₂ are independently selected from the group of C₇-C₁₈ alkyl, C₇-C₁₈ alkenyl, or C₇-C₁₈ alkynyl and R₃ is optionally substituted C₇-C₁₈ alkyl, C₇-C₁₈ alkenyl, or C₇-C₁₈ alkynyl.

4. The pharmaceutical composition of claim 3, or salt or isomer thereof, wherein n is 3 or 4.

5. The pharmaceutical composition of claim 1, or salt or isomer thereof, wherein R₁ or R₂ are independently selected from the group consisting of C₇-C₁₂ alkyl, C₇-C₁₂ alkenyl, and C₇-C₁₂ alkynyl, and R₃ is optionally substituted C₇-C₁₂ alkyl, C₇-C₁₂ alkenyl, or C₇-C₁₂ alkynyl, and n is 3, 4, 5, 6, 7, or 8, optionally wherein R₁ and R₂ are independently selected from the group consisting of C₇-C₁₂ alkyl, C₇-C₁₂ alkenyl, and C₇-C₁₂ alkynyl and R₃ is optionally substituted C₇-C₁₂ alkyl, C₇-C₁₂ alkenyl, or C₇-C₁₂ alkynyl and n is 3, 4, 5, 6, 7, or 8.

6. The pharmaceutical composition of claim 5, or salt or isomer thereof, wherein n is 3 or 4.

7. The pharmaceutical composition of claim 1, or salt or isomer thereof, wherein R₁ is selected from the group consisting of C₇-C₁₀ alkyl, C₇-C₁₀ alkenyl, and C₇-C₁₀ alkynyl, R₂ is the same as R₁, and R₃ is optionally substituted C₇-C₁₂ alkyl, C₇-C₁₂ alkenyl, or C₇-C₁₂ alkynyl and n is 3, 4, 5, 6, or 7.

8. The pharmaceutical composition of claim 7, or salt or isomer thereof, wherein n is 3 or 4.

9. The pharmaceutical composition of claim 1, or salt or isomer thereof, wherein R₁ and R₂ are independently C₈-C₁₂ alkyl, R₃ is optionally substituted C₈-C₁₂ alkyl, and n is 3 or 4.

10. The pharmaceutical composition of claim 1, or salt or isomer thereof, wherein R₁ is C₈-C₁₂ alkyl, R₂ is the same as R₁, R₃ is optionally substituted C₈-C₁₂ alkyl, and n is 3 or 4.

11. The pharmaceutical composition of claim 1, or salt or isomer thereof, wherein R₁, R₂, and R₃ are independently an alkyl selected from the group consisting of heptane, octane, nonane, decane, undecane, and dodecane.

12. The pharmaceutical composition of claim 1, or salt or isomer thereof, wherein one or more of R₁, R₂, and R₃ are independently an alkenyl selected from the group consisting of hept-1-ene, hept-2-ene, hept-3-ene, oct-1-ene, oct-2-ene, oct-3-ene, oct-4-ene, non-1-ene, non-2-ene, non-3-ene, non-4-ene, non-5-ene, dec-1-ene, dec-2-ene, dec-3-ene, dec-4-ene, dec-5-ene, dec-6-ene, undec-1-ene, undec-2-ene, undec-3-ene, undec-4-ene, undec-5-ene, undec-6-ene, undec-7-ene, dodec-1-ene, dodec-2-ene, dodec-3-ene, dodec-4-ene, dodec-5-ene, dodec-6-ene, dodec-8-ene, and an alkenyl group comprising two or more double bonds.

13. The pharmaceutical composition of claim 1, or salt or isomer thereof, wherein one or more of R₁, R₂, and R₃ are independently an alkynyl selected from the group consisting of hept-1-yne, hept-2-yne, hept-3-yne, oct-1-yne, oct-2-yne, oct-3-yne, oct-4-yne, non-1-yne, non-2-yne, non-3-yne, non-4-yne, non-5-yne, dec-1-yne, dec-2-yne, dec-3-yne, dec-4-yne, dec-5-yne, dec-6-yne, undec-1-yne, undec-2-yne, undec-3-yne, undec-4-yne, undec-5-yne, undec-6-yne, undec-7-yne, dodec-1-yne, dodec-2-yne, dodec-3-yne, dodec-4-yne, dodec-5-yne, dodec-6-yne, dodec-8-yne, and an alkynyl group comprising two or more triple bonds.

14. A pharmaceutical composition comprising a reversible zwitterionic lipid selected from among: 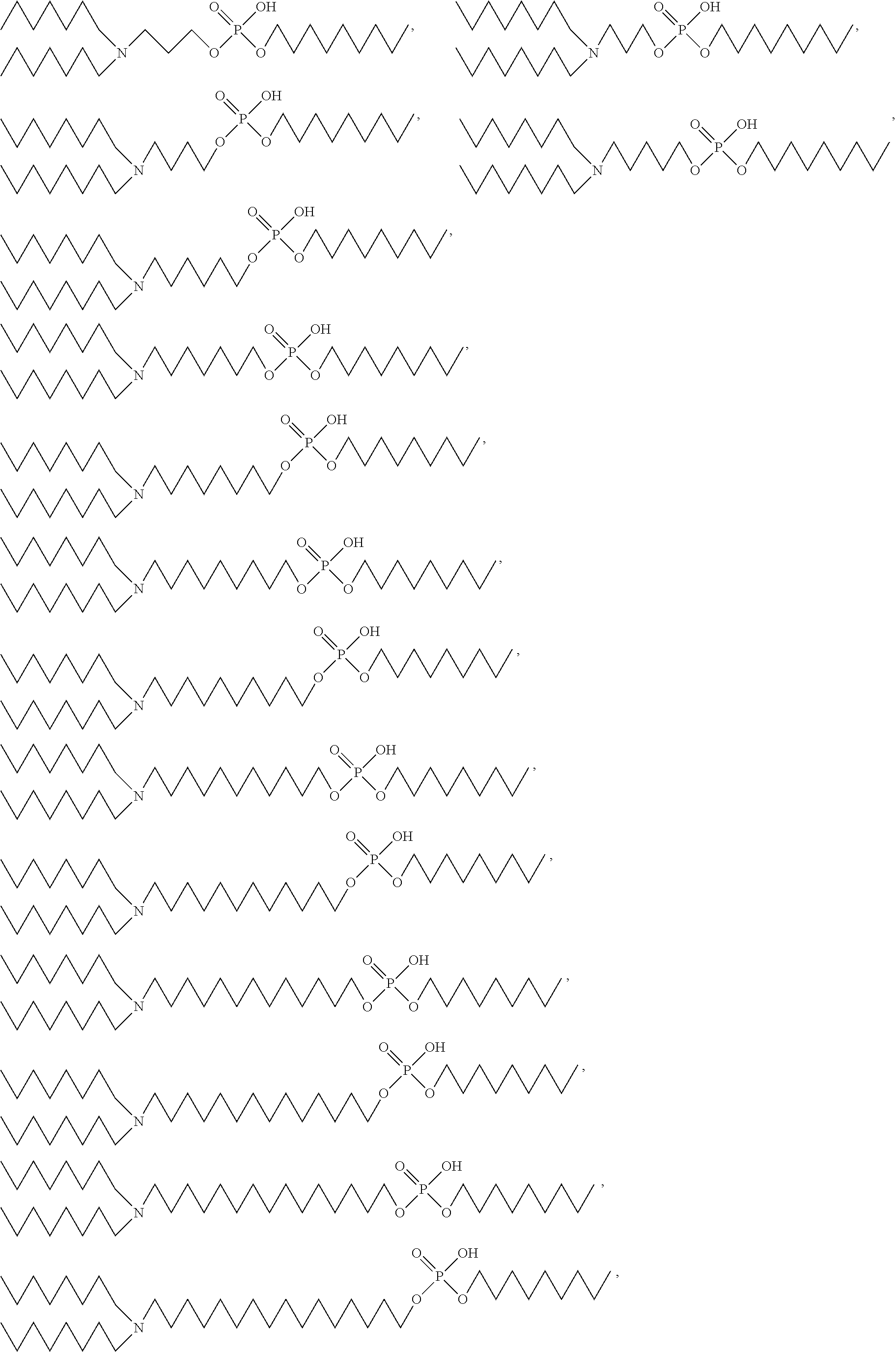 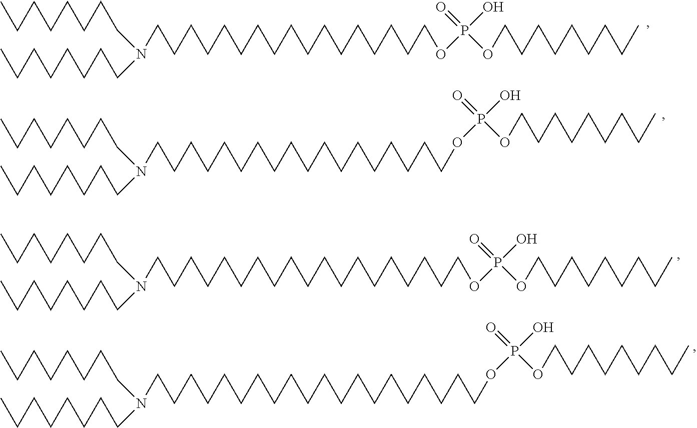
and salts and isomers thereof.

15. A composition selected from among the following:
A pharmaceutical composition comprising a reversible zwitterionic lipid selected from among: 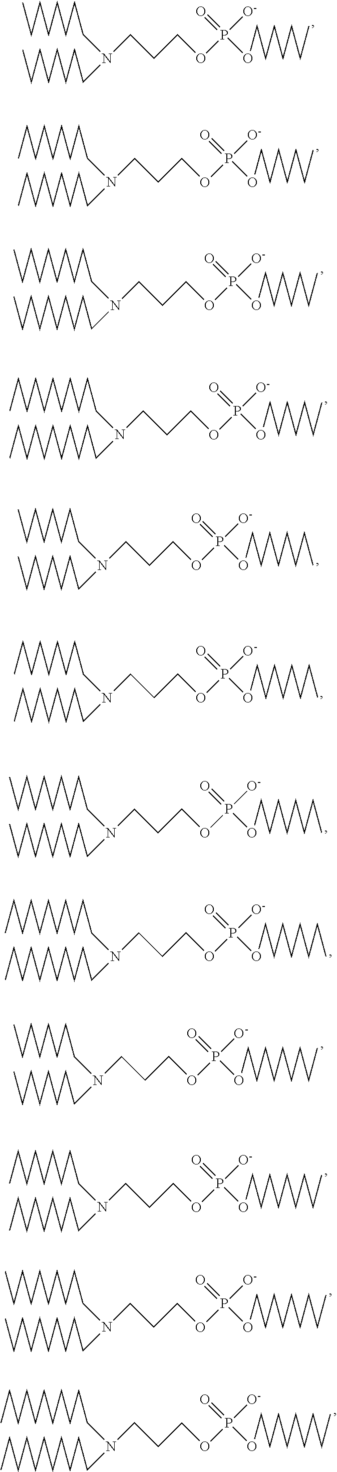 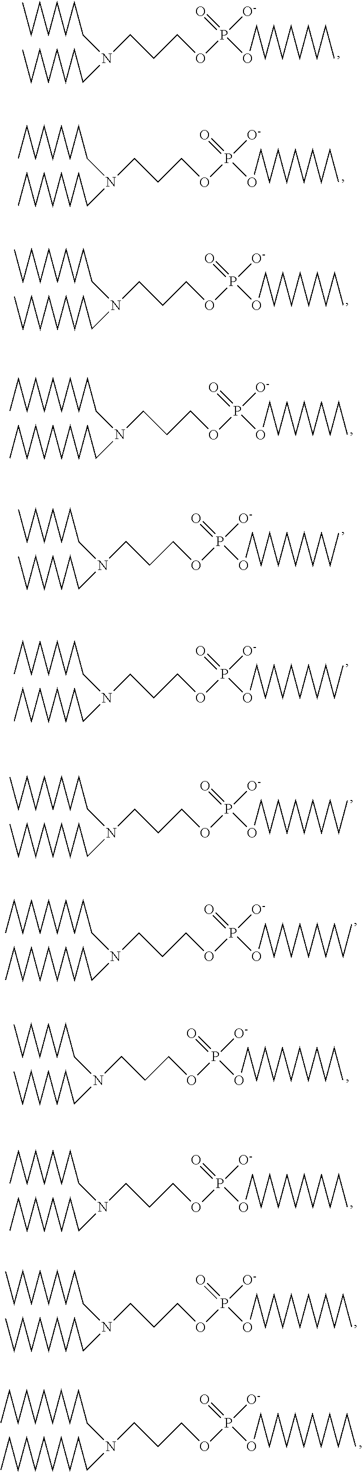
and salts and isomers thereof;
A pharmaceutical composition comprising a reversible zwitterionic lipid selected from among: 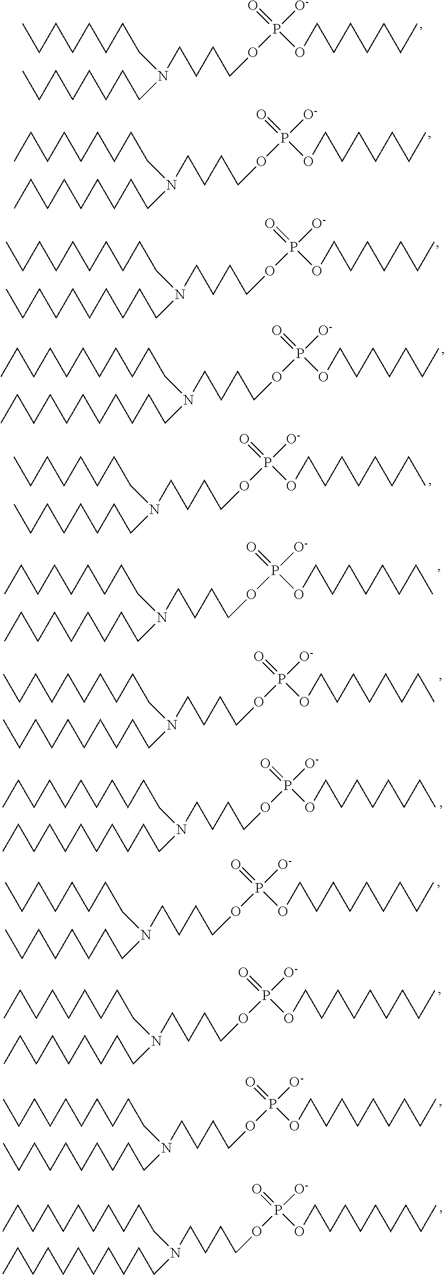 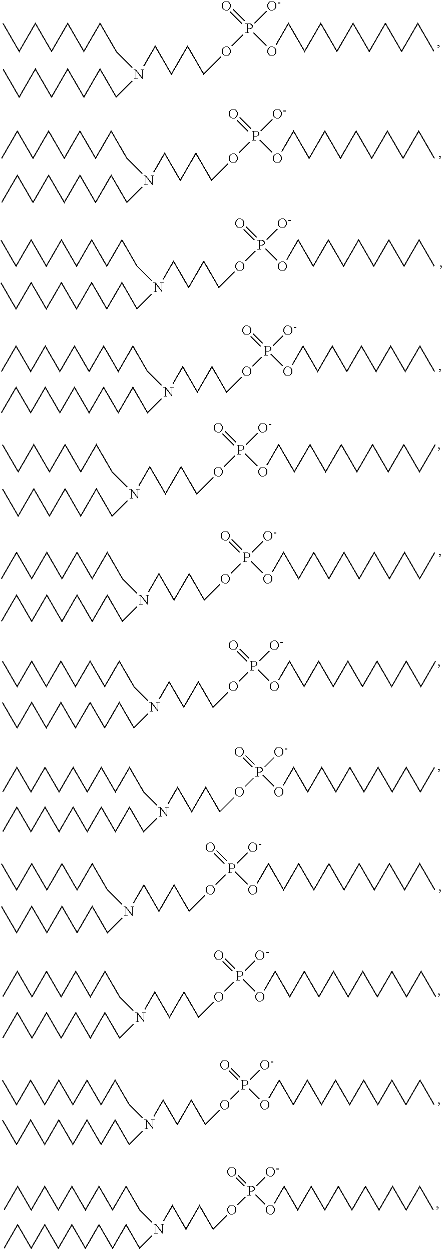
and salts and isomers thereof;
A pharmaceutical composition comprising a reversible zwitterionic lipid selected from among: 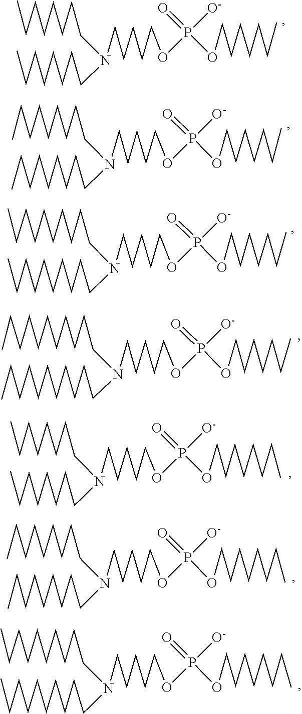 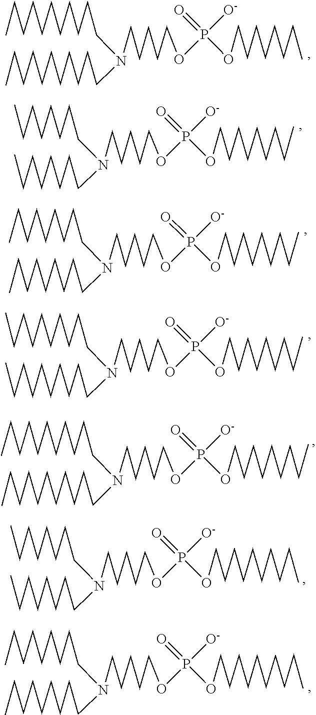 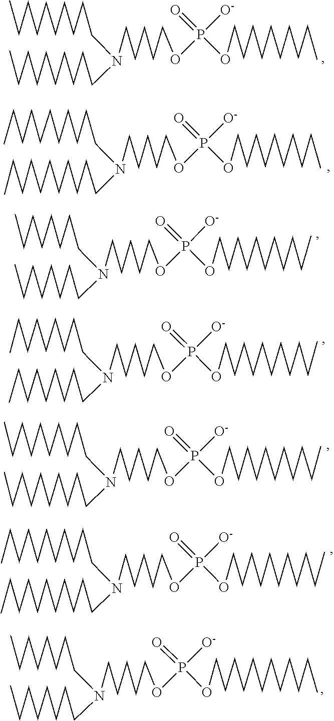 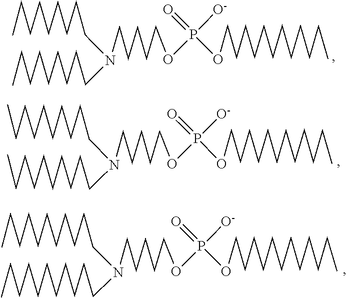
and salts and isomers thereof;
A pharmaceutical composition comprising a reversible zwitterionic lipid selected from among: 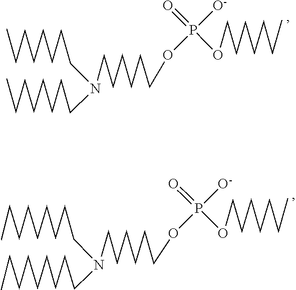 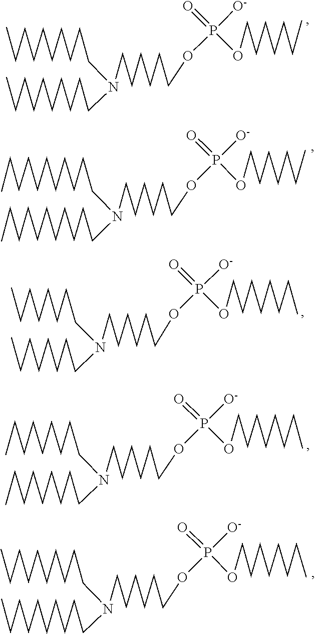 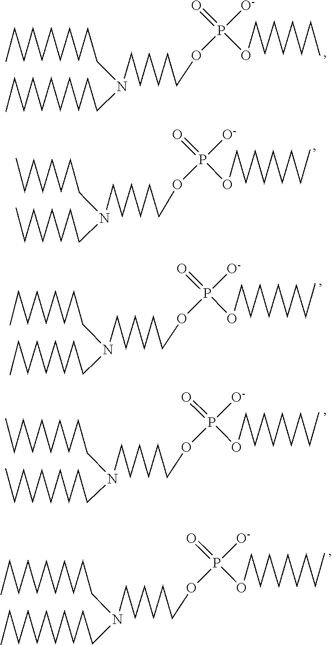 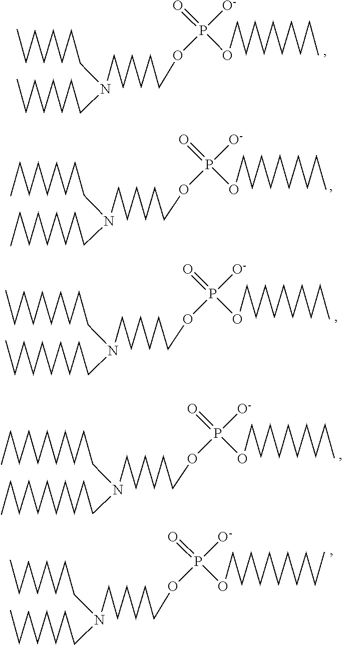 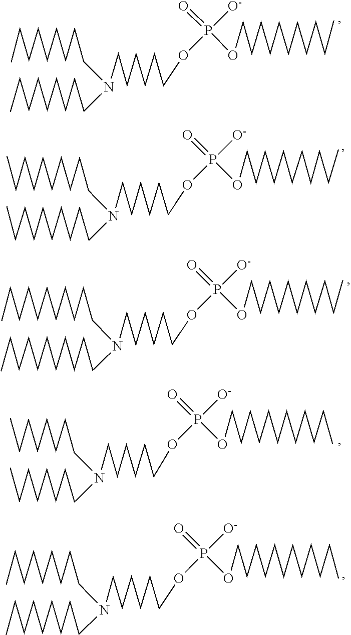 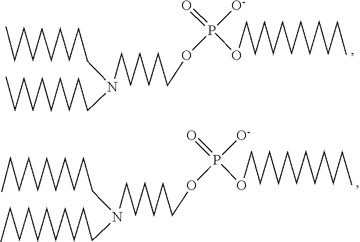
and salts and isomers thereof;
A pharmaceutical composition comprising a reversible zwitterionic lipid selected from among: 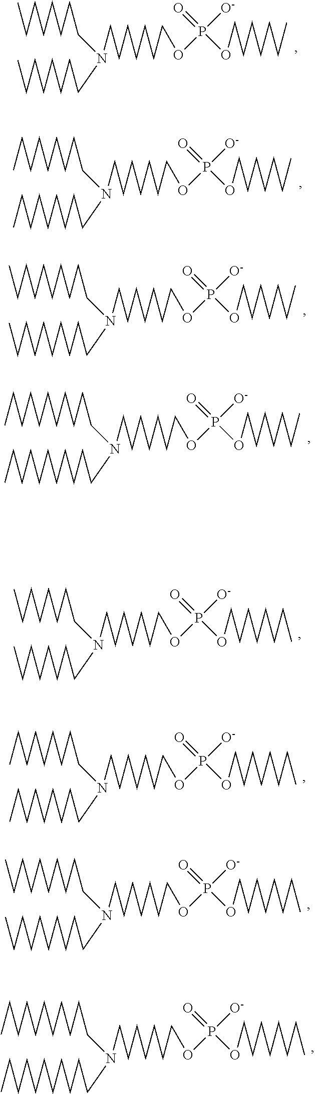 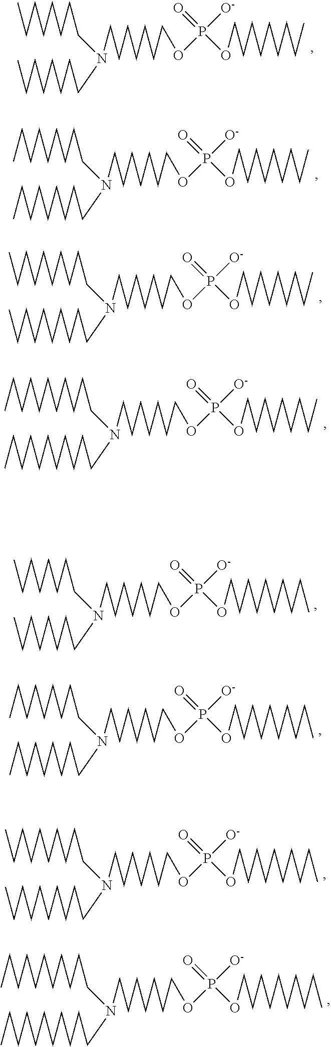 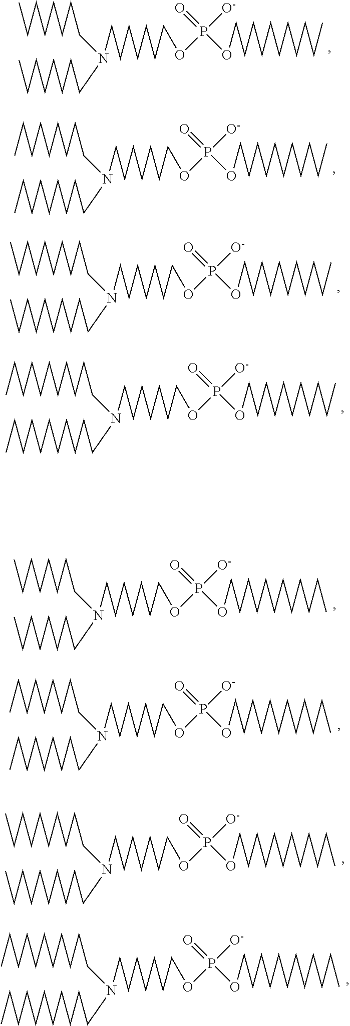
and salts and isomers thereof;
A pharmaceutical composition comprising a reversible zwitterionic lipid selected from among: 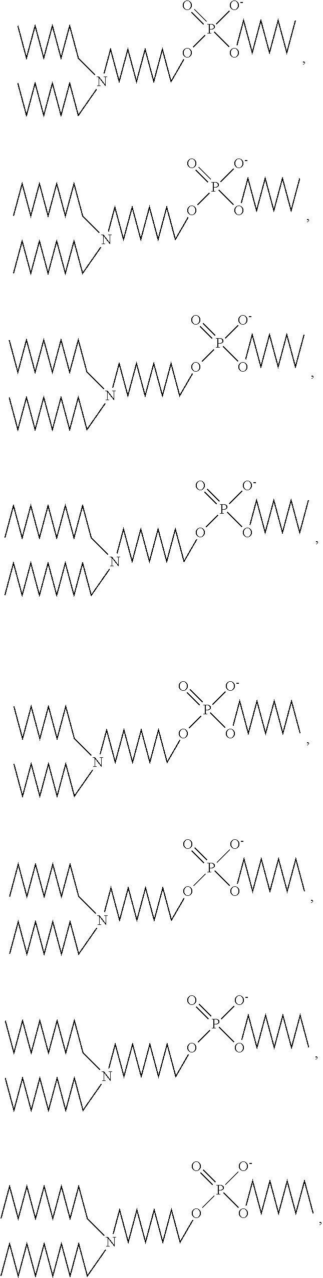 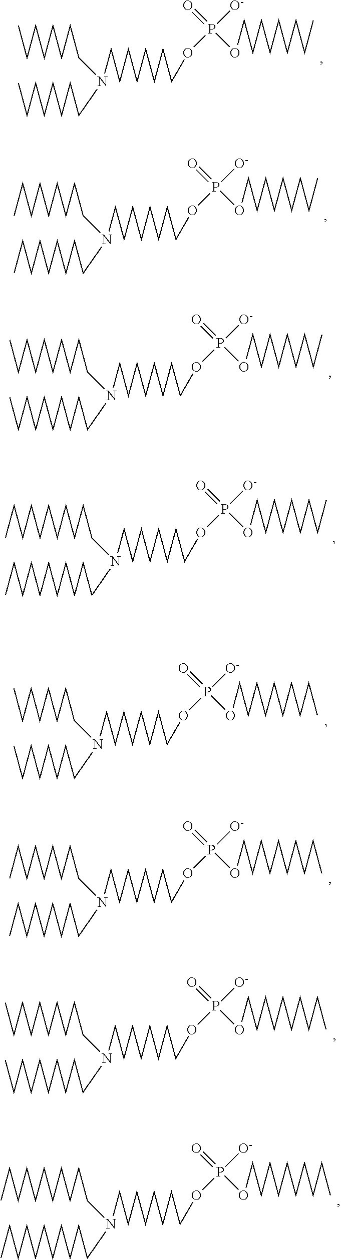 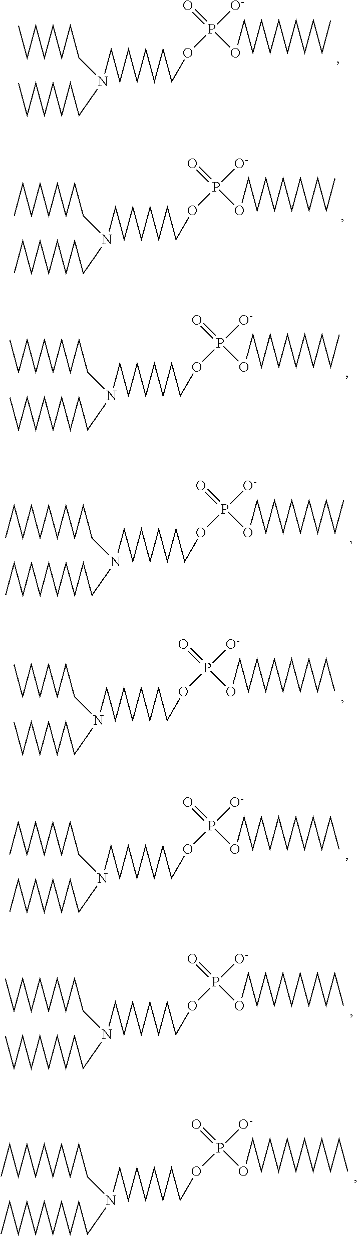
and salts and isomers thereof;
A pharmaceutical composition comprising a reversible zwitterionic lipid selected from among: 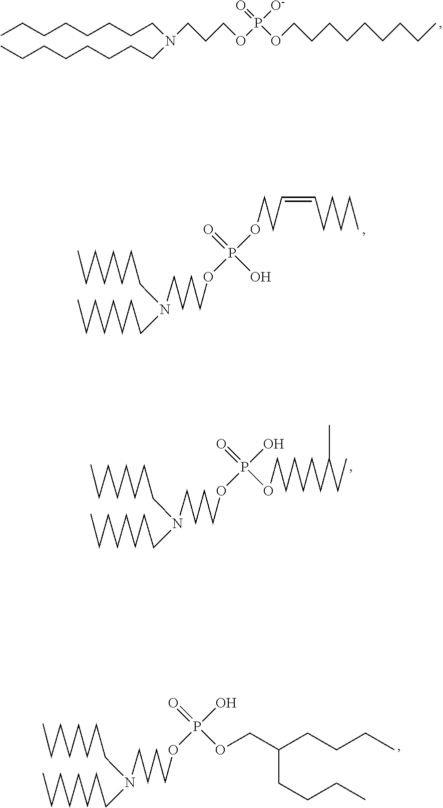
and salts and isomers thereof;
A reversible zwitterionic lipid selected from among: 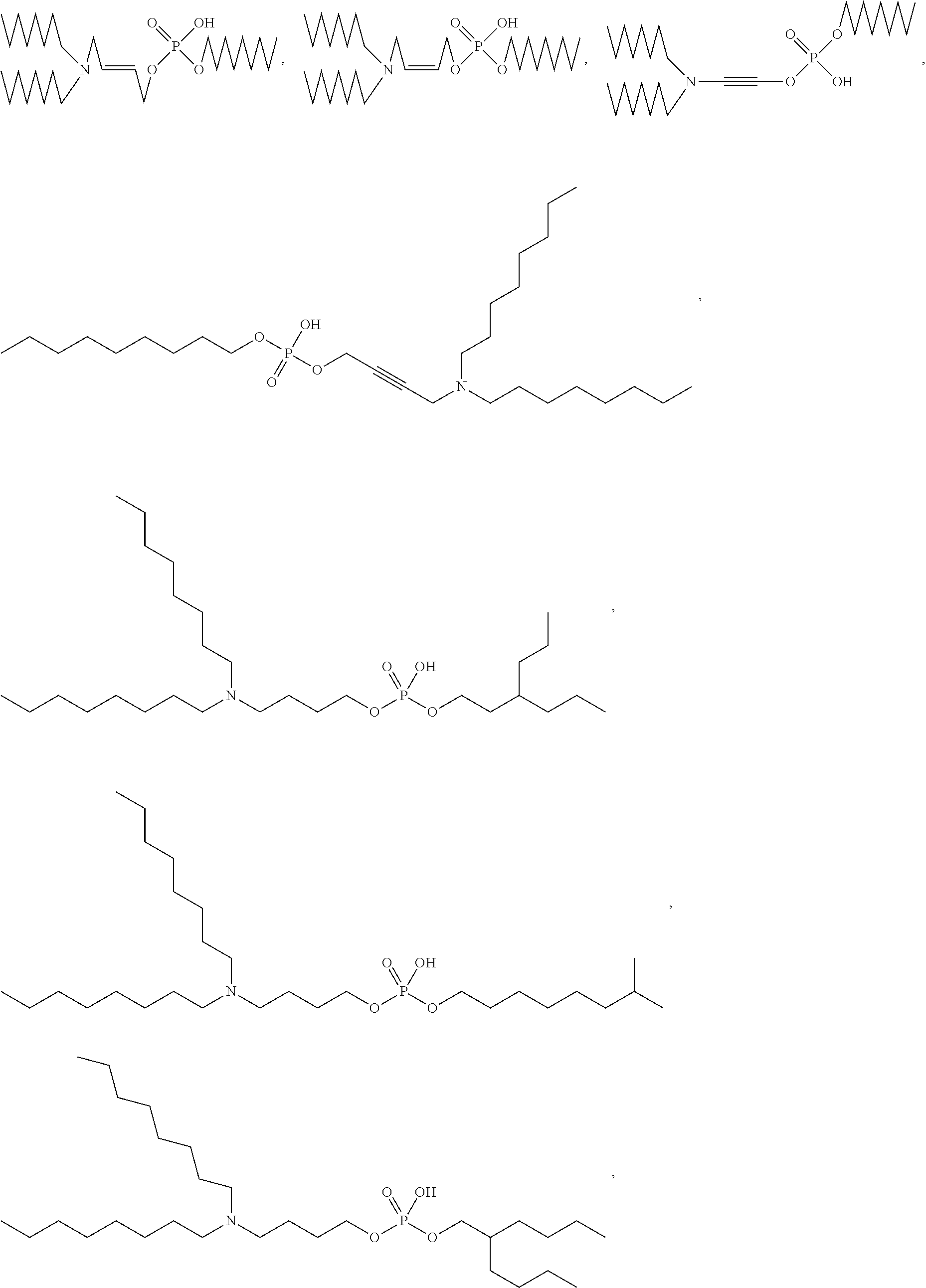 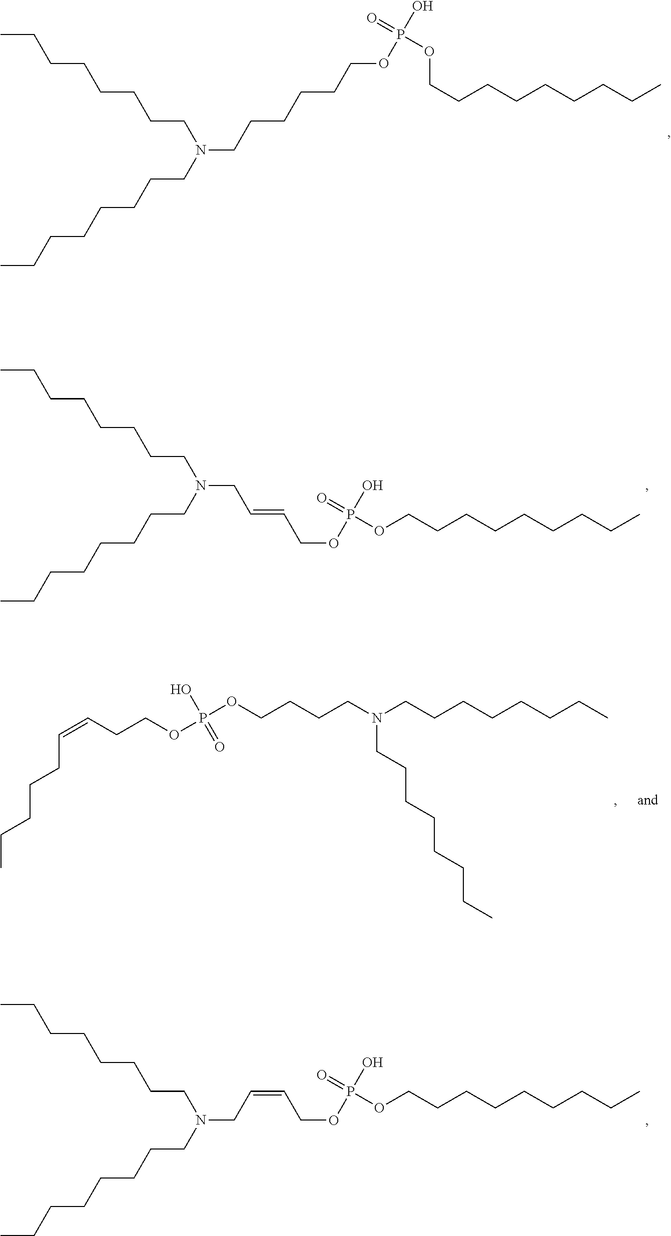
and salts and isomers thereof.

16-22. (canceled)

23. A lipid particle comprising a reversible zwitterionic lipid of claim 15.

24. The lipid particle of claim 23, further comprising a therapeutic agent.

25. The lipid particle of claim 24, wherein the therapeutic agent is a nucleic acid.

26. A pharmaceutical composition comprising a lipid particle of claim 23 and a pharmaceutically acceptable excipient, carrier, or diluent.
